(19) United States
(12) Patent Application Publication
(10) Pub. No.: US 2007/0105944 A1
(43) Pub. Date: May 10, 2007

(54) Taxane metabolites

(76) Inventor: S. Nilgun Comezoglu, Kendall Park, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 10/532,533

(22) PCT Filed: Feb. 5, 2003
(86) PCT No.: PCT/US03/03483

§ 371 Date: Apr. 25, 2005

Related U.S. Application Data
(60) Provisional application No. 60/421,405, filed on Oct. 25, 2002.

(51) Int. Cl. A61K 31/337; C07D 305/14

(52) U.S. Cl. 514/449; 549/510

(57) ABSTRACT

This invention relates to compounds of 3′-tert-butyl-3′-N-tert-butyloxycarbonyl-4-deacetyl-3′dephenyl-3′-N-debenzoyl-4-O-methoxycarbonyl-paclitaxel. The compounds of the invention are useful as therapeutic agents.

This application claims a benefit of priority from U.S. Provisional Application No. 60/421,405, the entire disclosure of which is herein incorporated by reference.

### FIELD OF THE INVENTION

This invention relates to compounds that are metabolites of 3′-tert-butyl-3′-N-tert-butyloxycarbonyl-4-deacetyl-3′-dephenyl-3′-N-debenzoyl-4-O-methoxycarbonyl-paclitaxel. The compounds of the invention are useful as therapeutic agents.

### BACKGROUND OF THE INVENTION

Paclitaxel is a natural product exacted from the bark of Pacific yew trees, Taxus brevifolia and the active constituent of the anticancer agent TAXOL®. It has been shown to have excellent anituimor activity in in vivo animal models, and recent studies have elucidated its unique mode of action, which involves abnormal polymerization of tubulin and disruption of mitosis. It is used clinically against a number of human cancers. It is an important cancer agent both therapeutically and commercially. Numerous clinical trials are in progress to expand and increase the utility of this agent for the treatment of human proliferative diseases. The results of TAXOL® clinical studies have been reviewed by numerous authors. A very recent compilation of articles by a number of different authors is contained in the entire issue of Seminars in Oncology 1999, 26 (1, Suppl 2). Other examples are such as by Rowinsky et al. in TAXOL®: A Novel Investigational Antimicrotubule Agent, J. Natl. Cancer Inst., 82: pp 1247-1259, 1990; by Rowinsky and Donehower in “The Clinical Pharmacology and Use of Antimicrotubule Agents in Cancer Chemotherapeutics,” Pharmac. Ther., 52;35-84, 1991; by Spencer and Faulds in “Paclitaxel, A Review of its Pharmacodynamic and Pharmacokinetic Properties and Therapeutic Potential in the Treatment of Cancer,” Drugs, 48 (5) 794-847, 1994; by K. C. Nicolaou et al. in “Chemistry and Biology of TAXOL®,” Angew. Chem., Int. Ed. Engl., 33: 15-44, 1994; by F. A. Holmes, A. P Kudelka, J. J. Kavanaugh, M. H. Huber, J. A. Ajani, V. Valero in the book “Taxane Anticancer Agents Basic Science and Current Status” edited by Gunda I. Georg, Thomas T. Chen, Iwao Ojima, and Dolotrai M. Vyas, 1995, American Chemical Society, Washington, DC, 31-57; by Susan G. Arbuck and Barbara Blaylock in the book “TAXOL® Science and Applications” edited by Mathew Suffness, 1995, CRC Press Inc., Boca Raton, Fla., 379-416; and also in the references cited therein.

A semi-synthetic analog of paclitaxel named docetaxel has also been found to have good antitumor activity and is the active ingredient of the commercially available cancer agent TAXOTERE®. See, Biologically Active Taxol Analogues with Deleted A-Ring Side Chain Substitutents and Variable C-2′ Configurations, J. Med. Chem., 34, pp 1176-1184 (1991); Relationships between the Structure of Taxol Analogues and Their Antimitotic Activity, J. Med. Chem, 34, pp 992-998 (1991). A review of the clinical activity of TAXOTERE® by Jorge E. Cortes and Richard Pazdur has appeared in Journal of Clinical Oncology 1995, 13(10), 2643 to 2655. The structures of pacitaxel and docetaxel are shown below along with the conventional numbering system for molecules belonging to the class; such numbering system is also employed in this application. 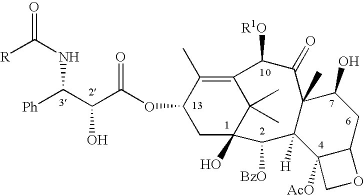
paclitaxel (TAXOL®): R=Ph; R′=acetyl
docetaxel (TAXOTERE®): R=t-butoxy; R′=hydrogen

Ample evidence that paclitaxel has no oral activity can be found within the following quote from PCT patent application WO98/53811 by inventors Samuel Broder, Kenneth L. Duchin and Sami Selim and the references cited within the quote, which says: “Paclitaxel is very poorly absorbed when administered orally (less than 1%); see Eiseman et al., Second NCI Workshop on Taxol and Taxus (September 1992); Suffness et.al. in TAXOL Science and Applications (CRC Press 1995). Eisemann et. al. indicate that paclitaxel has a bioavailability of 0% upon oral administration and Suffness et. al. report that oral dosing with paclitaxel did not seem possible since no evidence of antitumor activity was found on oral administration up to 160 mg mg/kg/day. Moreover, no effective method has been developed to enable the effective administration of oral paclitaxel (ie. a method of increasing the oral bioavailability of paclitaxel) or of other oral taxanes or paclitaxel analogs such as docetaxel which exhibit antitumor activity. For this reason, paclitaxel has not until now been administered orally to human patients, and certainly not in the course of treating paclitaxel-responsive diseases. Another report by J. Terwogt et. al. from The Lancet, Jul. 25, 1998, vol 352 page 285 also describes the low bioavailability of paclitaxel after oral dosing. In our own work, we have orally dosed paclitaxel to doses as high as 160 mg/kg/inj in murine (mouse) tumor models (sc M109) without signs of any efficacy and have concluded, like Suffness, that further dosing would not provide efficacy even though toxic doses were not reached. Furthermore, our own attempts to demonstrate activity for orally administered paclitaxel against human tumor xenografts implanted in either athymic mice or athymic rats have to date been unsuccessful.

This invention relates to C-4 methyl carbonate taxane analogs which have surpising oral activity and thus would have utility against proliferative diseases after oral administration. In particular, the invention relates to metabolites of a C-4 methyl carbonate taxane analog which have been identified through biotransformation and incubation from mouse, rat, dog, monkey and human liver microsomes. Some of the background art pertaining to this invention are shown below.

Certain taxane derivatives with modifications at the C-4 hydroxy group have been described in the art.

U.S. Pat. No. 5,808,102 to Poss et al and PCT published patent application WO 94/14787 contain descriptions of taxane analogs with modifications at the C-4 positions.

Gunda I. Georg et.al describe the synthesis of a C-4 ester analog in Tetrahedron Letters, 1994, 35(48) 8931-8934.

S. Chen et al. describe the synthesis of a C-4 cyclopropyl ester analog in Journal of Organic Chemistry 1994, 59(21), 6156-8.

U.S. Pat. No. 5,840,929 to Chen, Shu-Hui covering the C4 methoxy ether derivatives has issued on Nov. 24, 1998. A publication on the same topic has appeared:

Chen, Shu-Hui. First syntheses of C-4 methyl ether paclitaxel analogs and the unexpected reactivity of 4-deacetyl-4-methyl ether baccatin III. Tetrahedron Lett. 1996, 37(23), 3935-3939.

The following reference discusses a number of C-4 ester or carbonate analogs; Chen, Shu-Hui; Wei, Jian-Mei; Long, Byron H.; Fairchild, Craig A.; Carboni, Joan; Mamber, Steven W.; Rose, William C.; Johnston, Kathy; Casazza, Anna M.; et al. Novel C-4 paclitaxel (Taxol) analogs: potent antitumor agents. Bioorg. Med. Chem. Lett. 1995, 5(22), 2741-6.

The preparation of C-4 aziridinyl carbamate analogs has been described in: Chen, Shu-Hui; Fairchild, Craig; Long, Byron H. Synthesis and Biological Evaluation of Novel C-4 Aziridine-Bearing Paclitaxel (Taxol) Analogs. J. Med. Chem. 1995, 38(12), 2263-7.

The following papers describe reactions or transformations which are described as of potential for C-4 analog preparation:

A new method to modify the C-4 position of 10-deacetylbaccatin III. Uoto, Kouichi; Takenoshita, Haruhiro; Ishiyama, Takashi; Terasawa. Hirofumi; Soga, Tsunehiko. Chem. Pharm. Bull. 1997, 45(12), 2093-2095.

Samaranayake, Gamini; Neidigh, Kurt A.; Kingston, David G. I. Modified taxols, 8. Deacylation and reacylation of baocatin III. J. Nat. Prod. 1993, 56(6), 884-98.

Datta, Apurba; Jayasinghe, Lalith R; Georg, Gunda I. 4-Deacetyltaxol and 10-Acetyl-4-deacetyltaxotere: Synthesis and Biological Evaluation. J. Med. Chem. 1994, 37(24), 4258-60.

In spite of the abovementioned examples of C-4 analogs or methodology to prepare them, no evidence of orally active C-4 analogs has been supplied. Both TAXOL® and TAXOTERE® have no oral activity in human or animal models as mentioned in the following prior art described below on taxanes and oral modulators. Thus, the art to date does not suggest that C-4 taxanes should be different than other taxanes and therefore they should not be orally active. The art in no way specifically identifies any C-4 analogs which may have oral utility. The invention described in this patent application identifies metabolites of a novel C-4 analog which have oral activity.

The following references describe methods or possible methods for orally active taxanes.

Methods for administering taxanes in the presence of modulators have been been reported to increase the amount of taxanes in the plasma after oral administration: Terwogt, Jetske M. Meerum; Beijnen, Jos H.; Ten Bokkel Huinink, Wim W.; Rosing, Hilde; Schellens, Jan H. M. Coadministration of cyclosporin enables oral therapy with paclitaxel. Lancet (1998), 352(9124), 285.

Terwogt, Jetske M. Meerum; Malingre, Mirte M.; Beijnen, Jos H.; Huinink, Wim W. ten Bokkel; Rosing, Hilde; Koopman, Franciska J: Van Tellingen, Olaf; Swart, Martha; Schellens, Jan H. M. Coadministration of oral cyclosporin A enables oral therapy with paclitaxel. Clin. Cancer Res. (1999), 5(11), 3379-3384.

Hansel, Steven B. A method of making taxanes orally bioavailable by coadministration with cinchonine. PCT Int. Appl. WO 9727855 published Aug. 7, 1997.

Broder, Samuel; Duchin, Kenneth L.; Selim, Sami. Method and compositions for administering taxanes orally to human patients using a cyclosporin to enhance bioavailabiIity. PCT Int. Appl. WO 9853811 published Dec. 3, 1998. These reports contain no antitumor efficacy data but the presence of taxanes in the plasma is extrapolated to show their potential for anticancer utility.

At least one report of oral activity of prodrugs in preclinical animal models has appeared in the prior art: Scola, Paul M.; Kadow, John F.; Vyas, Dolatrai M. Preparation of paclitaxel prodrug derivatives. Eur. Pat. Appl. EP 747385 published Dec. 11, 1996. The oral bioavailability of the prodrug which had oral efficacy was not disclosed and no further reports of these compounds progressing to man have appeared.

Very recenty, an abstract describing a taxane analog (IDN-5109) with oral activity against tumors in mice was disclosed at the American Association of Cancer Researchers in Philadelphia in 1999. The reference for the abstract is: Pratesi G. Polizzi D, Totoreto M, Riva A, Bombardelli E, Zunino F: IDN5109 a new taxane active after oral administration. Proc Am Assoc Cancer Res 1999 40 Abs 1905, Istituto Nazionale Tumori, 20133 Milan and Indena SpA, 20139, Milan, Italy. The structure of this compound is quite different than compounds described in the present invention. Unlike the compounds encompassed by the present invention, IDN-5109 is derived from 14-betaydroxy baccatin III and has an acetate on the hydroxy group at the C-4 position.

Two references on the i.v. activity of this compound are included for completeness.

Nicoletti ML, Rossi C, Monardo C, Stura S, Morazzoni P, Bombardelli E, Valoti G, Giavazzi R.: Antitumor efficacy of a paclitaxel analogue, IDN5109, on human ovarian carcinoma xenografts with different sensitivity to paclitaxel Proc Am Assoc Cancer Res 1999 40 Abs 1910 [Evals+citations].

Polizzi, Donatella; Pratesi, Graziella; Tortoreto, Monica; Supino, Rosanna, Riva, Antonelia; Bombardelli, Ezio; Zunino, Franco. A novel taxane with improved tolerability and therapeutic activity in a panel of human tumor xenograhs. Cancer Res. 1999, 59(5), 1036-1040.

Paclitaxel is a highly schedule dependent drug that benefits traditionally from prolonged tumor exposure times. This relates to paclitaxel's mechanism of action as taxanes only recognize and bind to the polymerized state of tubulin which occurs only during a brief period of the cancer cell cycle. The currently used intravenous infusions (1-3 hours) are now readily accepted and efficacious and preclude the routine use of protracted (>24 hours) continuous schedules. However, an oral taxane may provide a compliant and cost effective way of accomplishing such extended duration of exposure. Recently, clinical utility has also been demonstrated using repetitive once weekly administrations of moderate (i.e., other than maximally tolerated) doses of TAXOL® and an oral taxane would be ideal for such protracted regimens. Other purported clinical indications for taxanes use (e.g., rheumatoid arthritis, multiple sclerosis) would also benefit from the availability of an oral taxane. An orally administered effective taxane would offer both an attractive alternative from the parenteral format of current clinical taxane usage, and a potential therapeutic advantage because of the many avenues of scheduling yet to be investigated.

Thus it is clear there is a great need to identify taxanes with both good oral bioavailability and good oral efficacy, which are comparable to paclitaxel administered parenterally.

### BRIEF DESCRIPTION OF THE DRAWINGS

FIG. 1 is a radiochromatogram for metabolites of [¹⁴C]Compound I in human liver microsomes following a one hour incubation. The scale of the vertical axis is relative abundance and the scale of the horizontal axis is time in minutes.

FIG. 2 is a radiochromatogram for metabolites of [¹⁴C]Compound I in monkey liver microsomes following a one hour incubation. The scale of the vertical axis is relative abundance nd the scale of the horizontal axis is time in minutes.

FIG. 3 is a radiochromatogram for metabolites of [¹⁴C]Compound I in rat liver microsomes following a one hour incubation. The scale of the vertical axis is relative abundance and the scale of the horizontal axis is time in minutes.

FIG. 4 is a radiochromatogram for metabolites of [¹⁴C]Compound I in mouse liver microsomes following a one hour incubation. The scale of the vertical axis is relative abundance and the scale of the horizontal axis is time in minutes.

FIG. 5 is a radiochromatogram for metabolites of [¹⁴C]Compound I in dog liver microsomes following a one hour incubation. The scale of the vertical axis is relative abundance and the scale of the horizontal axis is time in minutes.

FIG. 6 is a reconstructed HPLC radiochromatogram of [¹⁴C]Compound I after 1 hour incubation with mouse liver microsomes.

FIG. 7 is a reconstructed HPLC radiochromatogram of [¹⁴C]Compound I after 1 hour incubation with rat liver microsomes.

FIG. 8 is a reconstructed HPLC radiochromatogram of [¹⁴C]Compound I after 1 hour incubation with dog liver microsomes.

FIG. 9 is a reconstructed HPLC radiochromatogram of [¹⁴C]Compound I after 1 hour incubation with monkey liver microsomes.

FIG. 10 is a reconstructed HPLC radiochromatogram of [¹⁴C]Compound I after 1 hour incubation with human liver microsomes.

FIG. 11 is a radiochromatogram of 1 hour pooled plasma from two rats after a single 25 mg/kg oral dose of [¹⁴C]Compound I.

FIG. 12 is a radiochromatogram of 6 hour pooled plasma from two rats after a single 25 mg/kg oral dose of [¹⁴C]Compound I.

FIG. 13 is a radiocrhromatogram of 12 hour pooled plasma from two rats after a single 25 mg/kg oral dose of [¹⁴C]Compound I.

PIG. 14 is a radiochromatogram of 24 hour pooled plasma from two rats after a single 25 mg/kg oral dose of [¹⁴C]Compound I.

FIG. 15 is a radiochromatogram of 0-6 hour pooled bile from two rats atfer a single 25 mg/kg oral dose of [¹⁴C]Compound I.

FIG. 16 is a radiochromatogram of 6-12 hour pooled bile from two rats after a single 25 mg/kg oral dose of [¹⁴C]Compound I.

FIG. 17 is a radiochromatogram of 12-24 hour pooled bile from two rats after a single 25 mg/kg oral dose of [¹⁴C]Compound I.

FIG. 18 is a radiochromatogram of 0-24 hour pooled bile from two rats after a single 25 mg/kg oral dose of [¹⁴C]Compound I.

FIG. 19 is a radiochromatogram of 0-12 hour pooled urine from two rats after a single 25 mg/kg oral dose of [¹⁴C]Compound I.

FIG. 20 is a radiochromatogram of 12-24 hour pooled urine from two rats after a single 25 mg/kg oral dose of [¹⁴C]Compound I.

FIG. 21 is a radiochromatogram of 0-24 hour pooled urine from two rats after a single 25 mg/kg oral dose of [¹⁴C]Compound I.

### SUMMARY OF THE INVENTION

The invention relates to compounds that are metabolites of the anticancer compound 3′-tert-butyl-3′-N-tert-butyloxycarbonyl-4-deacetyl-3′-dephenyl-3′-N-debenzoyl-4-O-methoxycarbonyl-paclitaxel.

This invention also relates to pharmaceutical compositions comprising a metabolite of 3′-tert-butyl-3′-N-tert-butyloxycarbonyl-4-deacetyl-3′-dephenyl-3′-N-debenzoyl-4-O-methoxycarbonyl-paclitaxel or an optical or geometric isomer thereof; or a or a pharmaceutically acceptable salt, solvate or prodrug thereof.

Additionally, this invention provides for methods of treating disease comprising administering an effective amount of a metabolite of compound 3′-tert-butyl-3′-N-tert-butyloxycarbonyl-4-deacetyl-3′-dephenyl-3′-N-debenzoyl-4-O-methoxycarbonyl-paclitaxel possessing pharmacological activity.

This invention also provides for the use of metabolites of compound 3′-tert-butyl-3′-N-tert-butyloxycarbonyl-4-deacetyl-3′-dephenyl-3′-N-debenzoyl-4-O-methoxycarbonyl-paclitaxel or pharmaceutically acceptable salts, N-oxides and esters thereof for the manufacture of a medicament.

### DETAILED DESCRIPTION OF THE INVENTION

The present invention relates to metabolites of 3′-tert-butyl-3′-N-tert-butyloxycarbonyl-4-deacetyl-3′-dephenyl-3′-N-debenzoyl-4-O-methoxycarhonyl-paclitaxel represented by Formula I.

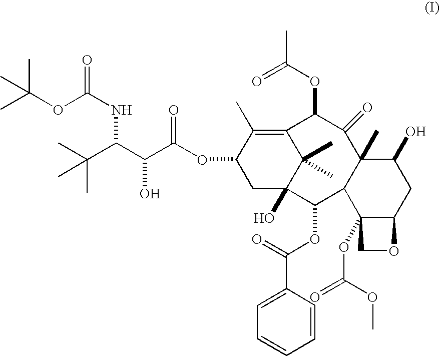

The metabolites disclosed herein are represented by compounds of formulas Ia and Ib:

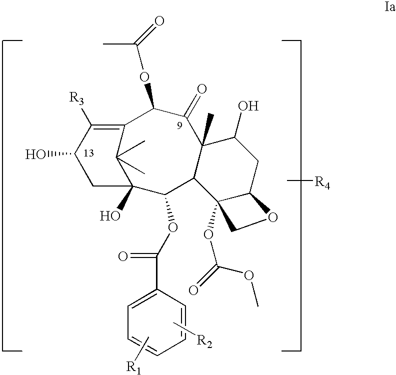

wherein the substituents are as defined in the following table:

| Metabolite | | | | | |
|---|---|---|---|---|---|
| Code | R₁ | R₂ | R₃ | R₄ | C9 |
| M1 | SG¹ | H | CH₃ | OH | C═O |
| M2 | SG¹ | OH | CH₃ | H | C═O |
| M4 & M5 | SG¹ | H | CH₃ | H | C═O |
| M6 | OCH₃ | OH | CH₃ | OH | C═O |
| M7 | OH | OCH₃ | CH₃ | OH | C═O |
| M8 | OH | H | CH₃ | OH | C═O |
| M8A | H | H | CH₃ | (OH)₂ | C═O |
| M9 | H | H | CH₃ | H | (CH)OH |
| M10 | H | H | CH₃ | OH | C═O |
| M11 | H | H | COOH | H | C═O |
| M12 | H | H | CH₃ | OH | C═O |
| M13 | H | H | CH₃ | H | C═O |

| | |
|---|---|
| ¹SG = glutathione conjugate | |
| 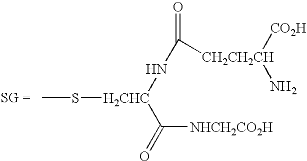 | |
| 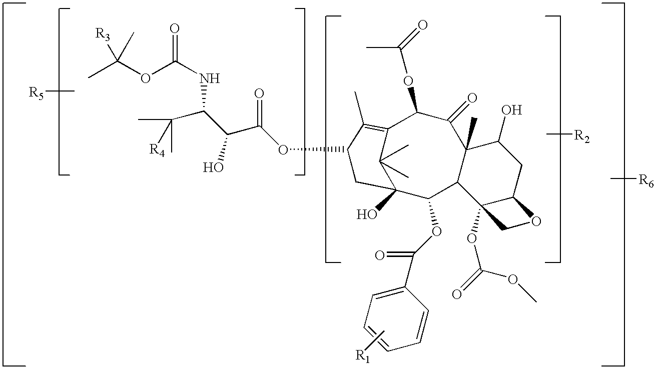 | Ib |

whererin the substituents are as defined in the following table:

| Metabolite | | | | | | |
|---|---|---|---|---|---|---|
| Code | R₁ | R₂ | R₃ | R₄ | R₅ | R₆ |
| M14¹ | H | H | CH₃ | CH₃ | H | 2O |
| M15 | SG⁶ | H | CH₃ | CH₃ | H | H |
| M16¹ | H | H | CH₃ | CH₃ | H | 2O |
| M17³ | H | H | CH₃ | CH₃ | OH | H |
| M18²³ | H | OH | CH₃ | CH₃ | OH | H |
| M19² | H | H | CH₃ | CH₃ | OH | H |
| M20⁴ | H | H | | | | H |
| M21⁴ | H | H | | | | H |
| M22 | H | H | CH₃ | COOH | H | H |
| M23³ | H | H | CH₃ | CH₃ | OH | H |
| M24⁵ | H | H | COOH | CH₃ | H | H |

¹the oxidation positions not definitive
²The oxidation position on the baccalin ring not definitive
³The oxidation position on the side chain not definitive
⁴Considered to be ring-closure products; see below for the structure of the side chain.
⁵R₃ and R₄ could be R₃ = CH₃ and R₄ = COOH
⁶SG = glutathione conjugate

Proposed structure of the side chain of M20 and M21

| | |
|---|---|
| M20 | 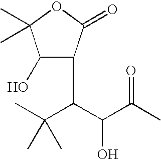 |
| | or |
| | 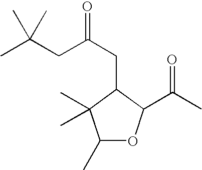 |
| M21 | 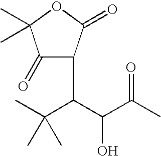 |
| | or |
| | 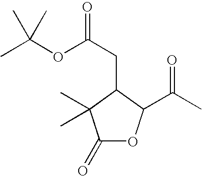 |

and by compounds of formulas Ic and Id;

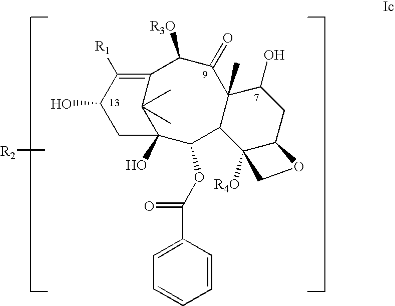

wherein the substituents are as defined in the following table:

| Metabolite | | | | | | |
|---|---|---|---|---|---|---|
| Code | R₁ | R₂ | R₃ | R₄ | C9 | C13 |
| M9 | CH₃ | H | CO(CH₃) | CO(OCH₃) | (CH)OH | (CH)OH |
| M10¹ | CH₃ | OH | CO(CH₃) | CO(OCH₃) | C═O | (CH)OH |
| M10A | CH₃ | H | H | CO(OCH₃) | C═O | (CH)OH |
| M11 | COOH | H | CO(CH₃) | CO(OCH₃) | C═O | (CH)OH |
| M10B¹ | CH₃ | OH | CO(CH₃) | CO(OCH₃) | C═O | (CH)OH |
| M10C | CH₃ | H | CO(CH₃OH) | CO(OCH₃) | C═O | (CH)OH |
| M12¹ | CH₃ | OH | CO(CH₃) | CO(OCH₃) | C═O | (CH)OH |
| M13 | CH₃ | H | CO(CH₃) | CO(OCH₃) | C═O | (CH)OH |
| M13A | CH₃ | H | H | CO(OCH₃) | C═O | C═O |
| M13B | CH₃ | OH | CO(CH₃) | H | C═O | (CH)OH |
| M13C² | CH₃ | H | CO(CH₃) | CO(OCH₃) | C═O | (CH)OH |
| M13D | CH₃ | H | CO(CH₃) | CO(OCH₃) | C═O | C═O |

| | |
|---|---|
| ¹The position of the oxidation is not definitive. | |
| ²M13C is considered to be 7-epi-isomer of M13. | |
| 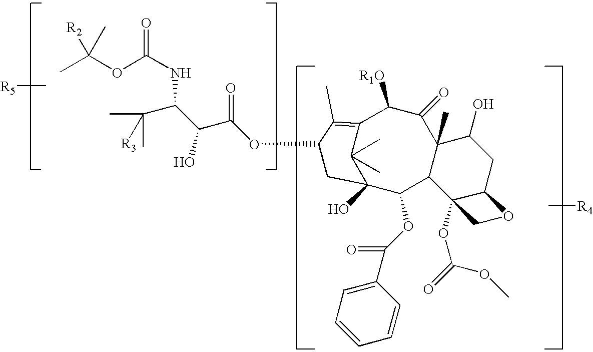 | 1d |

wherein the substituents are as defined in the following table:

| Metabolite | | | | | |
|---|---|---|---|---|---|
| Code | R₁ | R₂ | R₃ | R₄ | R₅ |
| M15B | CO(CH₃) | CH₂OH or | COOH or | H | H |
| | | COOH | CH₂OH | | |
| M17 | CO(CH₃) | CH₃ | CH₃ | H | OH |
| M18B | H | CH₃ | COOH | H | H |
| M19¹ | CO(CH₃) | CH₃ | CH₃ | H | OH |
| M19A | H | COOH | CH₃ | H | H |
| M22 | CO(CH₃) | CH₃ | COOH | H | H |
| M23¹ | CO(CH₃) | CH₃ | CH₃ | H | OH |
| M24 | CO(CH₃) | COOH or | CH₃ or | H | H |
| | | CH₃ | COOH | | |
| M23A | CO(CH₃) | CH₃ | CH₃ | OH or H | H or |
| | | | | | OH |
| M23² | CO(CH₃) | CH₃ | CH₃ | OH | H |
| M23C | CO(CH₃) | CH₃ | CH₃ | OH or H | H or |
| | | | | | OH |
| M26 | H | CH₃ | CH₃ | H | H |
| M23D² | CO(CH₃) | CH₃ | CH₃ | OH | H |
| M27³ | CO(CH₃) | CH₃ | CH₃ | H | H |

¹The oxidation on side chain, however, the position of the oxidation is not definitive
²The oxidation on the baccalin moiety, however, the position of the oxidation is not definitive
³7-cpi of Compound I

There are also disclosed compounds of the formula

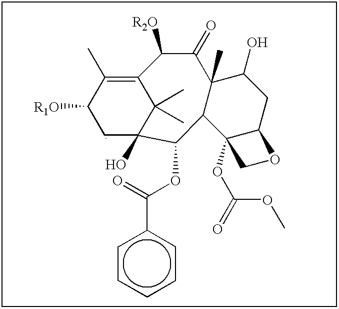

wherein the substituents are as defined in the following table:

| Metabolite | | |
|---|---|---|
| Code | R₁ | R₂ |
| M15A 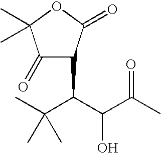 or | 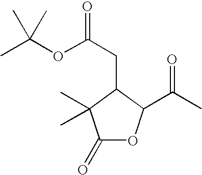 | CO(CH₃) |
| M18A 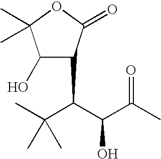 or | 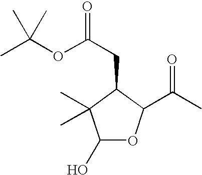 | H |
| M18C 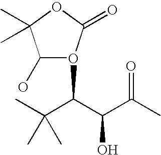 or | 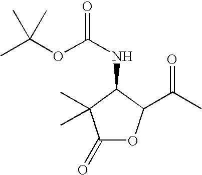 | H |
| M20/20B 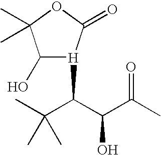 or | 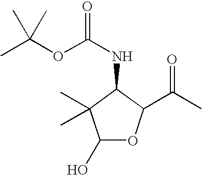 | CO(CH₃) |
| M21B 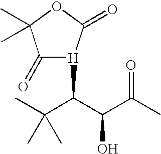 or | 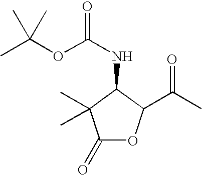 | CO(CH₃) |
| M25 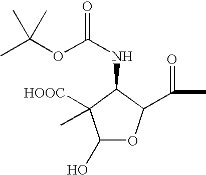 | | CO(CH₃) |

### DEFINITIONS

Listed below are definitions of various terms used to describe this invention. These definitions apply to the terms as they are used throughout this specification, unless otherwise indicated in specific instances.

As used herein, the term “bioavailable” means the extent to which a drug is absorbed into a living system and made available in the circulating blood of the living system. Methods to determine the bioavailability of drugs are well known to those of ordinary skill in the art.

As used herein, the phrase “sufficiently bioavailable to have a pharmacological effect” means that compounds of the invention are greater than 20 percent bioavailable, preferably greater than 30 percent bioavailable, and more preferably greater than 50 percent bioavailable.

As used herein, the term “pharmaceutically acceptable salt” refers to a salt prepared from a compound of the invention having a basic functional group, such as an amine, with a pharmaceutically acceptable nontoxic inorganic or organic acid. Suitable non-toxic acids include, but are not limited to, acetic, benzenesulfonic, benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, and p-toluenesulfonic acids. Salts formed with acids can be obtained, for example, with a compound of the invention having a basic functional group and an equivalent amount of a non-toxic acid to provide an acid addition salt. The reaction is typically carried out in a medium in which the acid addition salt precipitates or an aqueous medium followed by evaporation. The term “pharmaceutically acceptable salt” also refers to a salt prepared from a compound of the invention having an acidic functional group, such as a carboxylic acid functional group, and a pharmaceutically acceptable non-toxic inorganic or organic base. Suitable non-toxic bases include hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trilkylamines; dicyclohexylamine: tributyl amine; pyridine; N-methyl,N-ethylamine; diethylamine; triethylamine; mono, bis-, or tris-(2-hydroxy-lower alkyl amines), such as mono-, bis-, or tris-(2-hydroxyethyl)amine, 2-hydroxy-tert-butylamine, or tris-(hydroxytmethyl)methylamine, N, N,-di-lower alkyl-N-(hydroxy lower alkyl)-amines, such as N,N,-dimethyl-N-(2-hydroxyethyl)amine, or tri-(2-hydroxyethyl)amine; N-methyl-D-glucamine; and amino acids such as arginine, lysine, and the like. Salts formed with bases can be obtained, for example, with a compound of the invention having an acidic functional group and an equivalent amount of a non-toxic base. The reaction is typically carried out in a medium in which the salt precipitates or an aqueous medium followed by evaporation.

As used herein, the term “pharmaceutically acceptable acid neutralizing buffer” refers to a combination of a pharmaceutically acceptable non-toxic acid and a pharmaceutically acceptable nontoxic salt of an acid that when added to a solution provides a solution that is more resistant to change of pH, compared to a solution without the buffer, when acid or alkali is added to the solution. The term “pharmaceutically acceptable acid neutralizing buffer” also includes compounds, such as basic compounds, that when added to an acidic solution neutralizes the acid and increases the pH of the solution.

As used herein, the term “solvate” includes hydrated forms. In general, the solvated forms, with pharmaceutically acceptable solvents such as water, ethanol and the like, are equivalent to the unsolvated forms for purposes of this invention.

As used herein, the term “pro-drug” means a derivative of a compound that can hydrolyze, oxidize, or otherwise react under biological conditions (in vitro or in vivo) to provide a compound of the invention. For example, carboxylic esters are conveniently formed by esterifying carboxylic acid functionalities; if the compound of the invention includes an acid functional group it can be esterified to provide a pro-drug. Various pro-drugs are well known in the art. (For examples of pro-drugs, see Design of Prodrugs, edited by H. Bundgaard, Elsevier, 1985; Methods in Enzymology, vol. 42 p. 309-396, edited by K. Widder et al., Academic Press, 1985; A Textbook of Drug Design and Development, edited by Krosgaard-Larsen and H, Bundgaard, chapter 5. “Design and Application of Prodrugs,” by H. Bundgaard, p. 113-191, 1991; H. Bundgaard, Advanced Drug Delivery Reviews,” 8, 1-38, 1992; H. Bundgaard et al., Journal of Pharmaceutical Sciences, 77, 285, 1988; and N. Kakeya et al., Chem. Phar. Bull., 32, 692, 1984).

Utility and uses of the Compound IS or Compositions Thereof

The compounds of the invention are microtubule-stabilizing agents and, thus, can be used to treat a variety of cancer or other diseases of abnormal cell proliferation. The methods of the invention are particularly useful for administering one or more compounds of the invention, or a pharmaceutically acceptable salt, solvate, hydrate, or prodrug thereof, to a patient suffering from cancer or other hyperproliferative cellular disease. As used herein, the term “cancer” includes, but is not limited to, solid tumors and blood born tumors. The term cancer refers to disease of skin, tissues, organs, bone, cartilage, blood and vessels, The term “cancer” further encompasses primary and metastatic cancers.

Examples of cancers that can be treated with the methods of the invention include, but are not limited to, carcinoma, including that of the bladder, breast, colon, kidney, lung, ovary, pancreas, stomach, cervix, thyroid, and skin, including squamous cell carcinoma; hematopoietic tumors of lymphoid lineage, including, but not limited to, leukemia, acute lymphocytic leukemia, acute lymphoblastic leukemia, B-cell lymphoma, T-ceU lymphoma, Hodgkins lymphoma, non-Hodgkins lymphoma, hairy cell lymphoma, and Burketts lymphoma; hematopoietic tumors of myeloid lineage including, but not limited to, acute and chronic myelogenous leukemias and promyelocytic leukemia; rumors of mesenchymal origin including, but not limited to, fibrosarcoma, rhabdomyosarcoma, and osteosarcoma; other tumors including melanoma, seminoma, tetratocarcinoma, neuroblastoma, and glioma; tumors of the central and peripheral nervous system including, but not limited to, astrocytoma, neuroblastoma, glioma and schwannomas; and other tumors including, but not limited to, xenoderma, pigmentosum, keratoactanthoma, thyroid follicular cancer, and teratocarcinoma.

Exemplary classes of anticancer and cytotoxic agents include, but are not limited to, alkylating agents, such as nitorgen mustards, alkylsulfonates, nitrosoureas, ethylenimines, and triazenes; antimetabolites, such as folate antagonists, purine analogues, and pyrimidine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone anatagonists, octreotide acetate; microtubule-disruptor agents, such as ecteinascidins or their analogs and derivatives; microtubule-stabilizing agents such as paclitaxel (Taxol®), doceltaxel (Taxotere®); plant-derived products, such as vinca alkaloids, epipodophyllotoxins, taxanes; and topoisomerase inhibitors; prenyl-protein transferase inhibitors; and miscellaneous agents such as, hydroxyrea, procarbazine, mitotane, hexamethylmelamine, platinum coordination complexes such as cisplatin and carboplatin; and other agents used as anti-cancer and cytotoxic agents such as biological response modifiers, growth factors; immune modulators, and monoclonal antibodies. Compounds of the invention may also be used in conjunction with radiation therapy.

Representative examples of these classes of anti-cancer and cytotoxic agents include, but are not ltnited to, mechlorethamine hydrochlordie, cyclophosphamide, chlorambucil, melphalan, ifosfamide, busulfan, carmustin, lomustine, semustine, streplozocin, thiotepa, dacarbazine, methotrexate, thioguanine, mercaptopurine, fludarabine, pentastatin, cladribin, cytarabine, fluorouracil, doxorubicin hydrochloride, daunorubicin, idarubicin, bleomycin sulfate, mitomycin C, actinomycin D, safracins, saframycins, quinocarcins, discodermolides, vincristine, vinblastine, vinorelbine tartrate, etoposide, teniposide, paclitaxel, tamoxifen, estramustine, estramustine phosphate sodium, flutamide, buserelin, leuprolide, pteridines, diyneses, levamisole, aflacon, interferon, interleukins, aldesleukin, filgrastim, sargramostim, rituximab, BCG, tretinoin, irinolecan hydrochloride, betamethosone, gemcitabine hydrochloride, altretamine, and topoteca and any analogs or derivatives thereof.

Preferred members of these classes include, but are not limited to, paclitaxel, cisplatin, carboplatin, doxorubicin, carminoimycin, daunorubicin, aminopterin, methotrexate, methopterin, mitomycin C, ecteinascidin 743, porflromycin, 5-fluorouracil, 6-mercaptopurine, gemcitabine, cytosine arabinoside, podophyllotoxin or podophyllotoxin derivatives such as etoposide, etoposide phosphate or teniposide, melphalan, vinblastine, vincristine, leurosidine, vindesine, and leurosine.

Examples of anti-cancer and other cytotoxic agents include the following: cyclin dependent kinase inhibitors as found in WO 99/24416; and prenyl-protein transferase inhibitors as found in WO 97/30992 and WO 98/54966.

The compounds may also be administered with or after anti-cancer and cytotoxic agents that are neurotoxic, i.e., poisonous to the nervous system.

Compounds of the invention may also be formulated or co-administered with other therapeutic agents that are selected for their particular usefulness in administering therapies associates with the aforementioned conditions. For example, each of the compounds of the invention may be formulated with agents to prevent nausea, hypersensitivity, and gastric irritation, such as anti-emetics, and H₁ and H₂ antihistamines. The above therapeutic agents, when employed in combination with compounds of the invention, may be used in those amounts indicated in the Physicians' Desk Reference (PDR) or as otherwise determined by one of ordinary skill in the art.

As discussed above, compounds of the invention may be administered orally or intravenously (parenterally). It should be recognized that when compounds of the present invention are administered parenterally, it avoids the gastrointestinal system and overcomes any bioavailability concerns. However, in some instances such administration may be inconvenient and uncomfortable for the patient and provides other potential adverse effects. The compositions of this invention and the methods enable the oral route of administration to be used which is a significant advantage, particularly for human patients.

Pharmaceutical compositions and dosage forms suitable for oral administration can be presented as discrete dosage forms, such as, but not limited to, tablets (e.g., chewable tablets), caplets, capsules, powder in a sachet, enteric coated tablets, enteric coated beads, enteric coated soft gel capsules, and liquids (e.g., flavored syrups). Such dosage forms contain predetermined amounts of the active ingredient and may be prepared by methods of pharmacy well known to those skilled in the art (See Remington's Pharmaceutical Sciences, 18th ed., Mack Publishing, Easton Pa. (1990)).

In one embodiment, the compounds of the invention, or a pharmaceutically acceptable salt, solvate, or prodrug thereof, are administered as an enteric coated pill or capsule to delay release of the compound until after the pharmaceutically effective acid neutralizing buffer is administered. Enteric coated tablets and capsules are capsules coated with a substances that resist solution in a gastric fluid but disintegrate in the intestine.

Typical oral dosage forms are prepared by combining the active ingredients in an intimate admixture with at least one excipient according to conventional pharmaceutical compounding techniques. Excipients can take a wide varety of forms depending on the form of preparation desired for administration. For example, excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents. Examples of excipients suitable for use in oral liquid dosage forms include, but are not limited to, water, glycols, oils, alcohols, flavoring agents, preservatives, and coloring agents.

Tablets and capsules represent convenient pharmaceutical compositions and oral dosage forms, in which case solid excipients are employed. If desired, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalliue cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dexitrates, kaolin, mannitol, silicic acid, sorbitol, starch, pre-gelatinized starch, and mixtures thereof. The binder or filler in pharmaceutical compositions and dosage forms of the invention is typically present in from about 50 to about 99 weight percent of the pharmaceutical composition or dosage form.

Disintegrants are used in the pharmaceutical compositions and dosage forms of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form the pharmaceutical compositions and solid oral dosage forms of the invention. The amount of disintegrant used varies based upon the type of formulation, and is readily discernible to those of ordinary skill in the art. Typical pharmaceutical compositions and dosage forms comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polactilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

The pharmaceutical compositions and dosage forms may further comprise one or more compounds that reduce the rate by which an active ingredient will decompose. Such compounds, which are referred to herein as “stabilizers,” include, but are not limited to, antioxidants such as ascorbic acid and salt buffers.

Solutions for oral administration represent another convenient oral dosage form, in which case a solvent is employed. Liquid oral dosage forms are prepared by combining the active ingredient in a suitable solvent to form a solution, suspension, syrup, or elixir of the active ingredient in the liquid.

The solutions, suspensions, syrups, and elixirs may optionally comprise other additives including, but not limited to, glyceria, sorbitol, propylene glycol, sugars, flavoring agents, and stabilizers.

The magnitude of the therapeutic dose of the desired compounds of the invention, or a pharmaceutically acceptable salt, solvate, or prodrug thereof, will typically vary with the specific disease and severity of the disease being treated. The dose, and perhaps the dose frequency, may also vary according to age, body weight, response, and the past medical history of the patient. Suitable dosing regimens can be readily selected by those skilled in the art with due consideration of such factors. Typically, the compounds of the invention, or a pharmaceutically acceptable salt, solvate, or prodrug thereof, is orally administered in a total amount of about 0.01 to about 200 mg/kg/day, preferably from about 0.05 to about 50 mg/kg/day as a single dose or in divided doses such as from 1 to about 4 times per day. Preferably the compounds are administered in a dose less than about 100 mg/kg/day, more preferably in a dose less than 50 mg/kg/day in a single dose or in 1 to about 4 divided doses.

The invention also encompasses pharmaceutical unit dosage forms of the desired buffer comprising about 1 to about 200 mg/unit, preferably about 5 to about 100 mg/unit, and more preferably about 5 to about 50 mg/unit. Similarly, liquid unit doses of the buffer encompassed by the invention include about 1 to 200 mg/unit, preferably about 5 to 100 mg/unit, and more preferably about 5 to 50 mg/unit, dissolved in about 50 mL to 300 mL of a solvent, preferably about 100 mL to 200 mL of a solvent, and more preferably about 110 mL to 150 mL of a solvent.

The present invention is also directed to methods of treating cancer and other hyperproliferative diseases in patients comprising administering to the patient a therapeutically effective amount of one or more compounds of the invention. The compounds of the invention may be administered intravenously or orally. Preferably, the compounds of the invention are administered with one or more additional agents to prevent nausea, hypersensitivity, or gastric irritation such as an anti-emetic or an H₁ or H₂ antihistamine.

The amount of a compound of the invention administered by each IV infusion, or orally, or both may be determined by one of ordinary skill in the art, and includes exemplary dosage amounts for a human of from about 0.01 mg/kg/day to about 200 mg/kg/day, preferably form about 0.05 mg/kg/day to about 50 mg/kg/day, which may be administered in a single dose or in the form of individual divided doses, such as from 1 to about 4 times per day. Preferably, the compounds are administered in a dosage of less than about 100 mg/kg/day, and more preferably less than about 50 mg/kg/day in a single dose or in 1 to about 4 divided doses. It will be understood that the specific dose level and frequency of dosage for any particular subject may be varied and will depend upon a variety of factors including the activity of the specific compound employed, the metabolic stability and length of action of that compound, the species, age, body weight general health, sex and diet of the subject, the mode and time of administration, rate of excretion, drug combination, and severity of the particular condition. Preferred subjects for treatment include animals, most preferably mammalian species such as humans, and domestic animals such as dogs, cats and the like, subject to the aforementioned disorders.

Typically the compounds of the invention are administered until the patient shows a response, for example, a reduction in tumor size, or until dose-limiting toxicity is reached. One or ordinary skill in the art will readily know when a patient shows a response or when dose limiting toxicity is reached. The common dose-limiting toxicites associated with compounds of the invention include, but are not limited to, fatigue, arthralgiz/myalgia, anorexia, hypersensitivity, neutropenia, thrombocytopenia, and neurotoxicity.

By way of illustration, and without serving as limitations in any way, the following examples serve to illustrate the practice of the invention.

### EXAMPLES

The following abbreviations are used in the examples:

### List of Abbreviations

| | |
|---|---|
| CH₃CN | Acetonitrile |
| DLM | Dog Liver Microsomes |
| DPM | Disintegrations per minute |
| 7-HC | 7-Hydroxycoumarin |
| 7-EC | 7-Ethoxycoumarin |
| HLM | Human Liver Microsomes |
| LC/RAM/(+)ESI-MS | Liquid chromatography/radioactivity monitoring/ |
| | positive electrospray ionization-mass |
| | spectrometry |
| LSC | Liquid Scintillation Counting |
| CH₃OH | Methanol |
| M | Metabolite |
| MLM | Mouse Liver Microsomes |
| MKLM | Monkey Liver Microsomes |
| MS/MS | Mass spectrometry/mass spectrometry |
| m/z | mass to charge ratio |
| NADPH | β-Niacinamide adenine dinucleotide phosphate, |
| | reduced form |
| NC1 | Negative control 1 (No NADPH) |
| NC2 | Negative control 2 (Heat-inactivated microsomes) |
| PA | Peak area |
| PC | Positive control |
| RLM | Rat Liver Microsomes |
| ROI | Region of interest |
| RPM | Revolutions per minute |
| Rₜ | Retention time |
| TA | Test Article ([¹⁴C]Compound I) |

### Example 1

Preparation of [⁴C]-and [¹³C]Compound I Stock Solution in Acetonitrile (2 mM)

[¹⁴C] Compound I in Acetonitrile (2 mM)

The stock solution was prepared by dissolving 1.06 mg of [¹⁴C]Compound I(Lot No. 001; 26.1 μCi/mg) in 0.627 mL of CH₃CN. The concentration of the solution was 2 mM (2 μmol/mL) containing −44 μCi of radioactivity in each mL. The solution was stored at −20° C. prior to use.

[¹³C]Compound I in Acetonitrile (2 mM)

The stock solution was prpared by dissolving 1.15 mg of [¹³C]Compound I(Lot No. 001) in 0.675 mL of CH₃CN. The concentration of the solution was 2 mM (2 μmol/mL). The solution was stored at −20° C. prior to use.

Incubation System

Each mL of the incubation mixture, in 100 mM of potassium phosphate buffer (pH 7.4), contained: 20 nmol of test article, 1 mg of microsomal protens, 3 μmol of NADPH and 10 μmol of magnesium chloride. The final concentrations were: test article, 20 μM; microsomal proteins, 1 mg/mL; NADPH, 3 mM; magnesium chloride, 10 mM; acetonitrile, 1% (v/v). 7-EC (100 μM) was used as the positive control for enzyme activity in the microsomes of all species. Two negative controls, one without NADPH and the other using heat-treated microsomes (5 min in boiling water), were carried out to determine the stability of the test article. The total volume of incubation mixture was 5 mL each for the test article incubations and 1 mL each for the positive and negative controls.

Incubation and Sample Processing

Incubations were carried out aerobically at 37° C. with constant shaking. Incubation mixtures were prepared at 0-4° C. (on ice). Reaction was initiated by incubating the mixture in a 37° C. water bath. Incubation time was 1 hr for all samples. At the end of incubation, duplicate aliquots (10 μL) of the incubation mixture of the negative controls and the test article incubations were taken for liquid scintillation counting of radioactivity. The remaining incubation mixture was transferred to a 13-mL conical centrifuge tubes. The incubation vial was rinsed with an equal volume of methanol and the rinse was combined with the incubation mixture The combined incubation mixture and the rinse were vortexed and centrifuged at 2000 rpm for 10 min to remove precipitated proteins. Duplicate aliquots (10 μL) of the supernatant were taken for liquid scintillation counting. The methanol extract was evaporated to dryness under nitrogen at −40° C. Residues were dissolved in a small volume of a methanol/water (1:1, v/v) mixture. The reconstituted solutions were stored at −20° C. prior to analysis. Upon frozen storage, samples yielded a large amount of precipitates. Therefore, the entire concentrate was diluted back to ˜10 mL with a methanol/water (1:1, v/v) mixture. These samples were used for HPLC analyses.

At 0 and 1 hr, a single aliquot (0.2 mL) of the positive control (7-EC) incubation mixture was transferred to a tube containing 0.2 mL of acetonitrile, vortexed and centrifuged. The supernatant was then stored at −20° C. for analysis by HPLC/UV.

Determination of Metabolite Profiles

The radiochemical purity of [¹⁴C]Compound I and the metabolite profiles of the compound in the methanol extract of liver microsomal incubations were determined by HPLC radiochromatograpby using the following system:

| | |
|---|---|
| HPLC System: | Waters Model 2690 Separation Module |
| UV Detector: | 996 PDA at 254-nm wavelength, 1.0 AUFS |
| Radioactivity | Raytest Ramona-5 Flow-through Radioactivity |
| Detector: | Monitor with a 200-μL glass cell |
| Integrator/ | Millennium 32 data system |
| Data System: | |
| Column: | Ace 5 C18, 4.6 × 250 mm, 5 μm, (Series |
| | #A3538) |
| Column | 30° C. |
| Temperature: | |
| Guard Column: | RP-8, 5 μm, 4.6 × 30 mm |
| Mobile Phase: | A: 0.01M CH₃COONH₄ (adjust pH to 3 |
| | with HCOOH), |
| | B: CH₃OH |
| Gradient System | 100% A, 3 min to 40% B, 30 min to 60% B (15 min), |
| 1: | 10 min to 65% B, 5 min to 100% B (9 min), 2 min |
| | to initial. (Total run time = 74 min) |
| Gradient System | 100% A, 3 min to 40% B, 30 min to 60% B (15 min), |
| 2: | 10 min to 65% B, 5 min to 100% B (15 min), 2 min |
| | to initial. (Total run time = 80 min). |
| Flow Rate: | 1 mL/min |
| Autosampler | 4° C. |
| Temperature: | |
| Fraction | Foxy 200 Fraction Collector |
| Collector: | |

Gradient System 1 was used to analyze all the HLM samples and a RLM sample. Gradient System 2 was used to analyze the remaining samples.

Fractions of the HPLC effluent were collected (0.25 min/fraction) into a 96-well deep luma plates and the collected fractions were dried using Savant Automatic SpeedVac system. Radioactivity was then determined with a Packard Top Count-NXT counter. Chromatographic system suitability was routinely performed to verify the retention time of Compound I.

LC/RAM/(+)ESI-MS and MS/MS Identification of Metabolites

Metabolites of [¹⁴C]Compound I in the methanol extract of liver microsomal incubation of each species were analyzed by LC/RAM((+)ESI-MS and MS/MS. The following HPLC system and conditions were used.

LC/MS Condition I

| | |
|---|---|
| HPLC System: | Waters 2690 Separations Module |
| UV Detector: | Waters 486 Tunnble Absorbance Detector |
| Radioactivity | Raytest Ramona-5 Flow-through Radioactivity |
| Detector: | Monitor with a 110-μL glass cell |
| Column: | Ace 5 C18, 4.6 × 250 mm, 5 μm (Series #A3538) |
| Column | 30° C. |
| Temperature: | |
| Guard Column: | RP-8, 5 μm, 4.6 × 30 mm |
| Mobile Phase: | A: 0.01M CH₃COONH₄ (adjust pH to 3 with |
| | HCOOH), |
| | B: CH₃OH |
| Gradient System | 100% A, 3 min to 40% B, 30 min to 60% B (15 min), |
| 1: | 10 min to 65% B, 5 min to 100% B (9 min), 2 min to |
| | initial. (Total run time = 74 min). |
| Flow Rate: | 1 mL/min |
| Autosampler | 4° C. |
| Temperature: | |

The HPLC system was interfaced to a Finnigan MAT TSQ-7000 High performance Quadrupole Mass Spectrometer using the following conditions:

| | |
|---|---|
| Mass Spectrometer: | Finnigan MAT TSQ-7000 High Performance |
| | Quadrupole Mass Spectrometer |
| Ionization Mode: | Positive Electrospray |
| Ion Spray (IS): | 4.5 kV |
| Capillary Temperature: | 240° C. |
| Sheath Gas Flow: | 80 psi |
| Auxiliary Gas Flow: | 20 mL/min |
| Collision Gas (CAD): | Argon |

[¹⁴C] and [¹³C] Compound I Mixture in Acetonitrile (2 mM)

The stock solution was prepared by mixing one pan of the 2-mM stock solution of [¹⁴C]Compound I in acetonitrile with one part of the 2-mM stock solution of [¹³C]Compound I in acetonitrile. The solution was stored at −20° C. prior to use.

[¹⁴C]Compound I was separately incubated with mouse, rat, dog, monkey and human liver microsoomes fortified with NADPH for 1 hr at 37° C. The incubation mixtures were extracted with methanol and the extacts were analyzed by HPLC radiochromatograpby and LC/RAM/(+)ESI-MS and MS/MS.

The following shows the metabolites identified from human, monkey, rat, mouse, and dog liver microsomes.

A total of 36 compounds including the parent drug were identified or characterized in the incubation mixtures of [¹⁴C]Compound I with human, monkey, rat, mouse, and dog liver microsomes. Metabolites M1 to M-24 were also identified and numbered in the bile duct cannulated (BDC) rat study. The metabolites other than M1 to M24 were numbered based on their appropriate R,'s, e.g., M10A, M10B, etc. It is noted that the stereochemistry of the 7-position was not definitive in some metabolites.

1. Metabolites Identified from Human Liver Microsome Incubation

Compound I

The stock solution used to spike the incubation mixture was tested to show the presence of the radioactive Compound I. The solution contained approximately 1:1 ratio of [¹⁴C]Compound I and [¹³C]Compound I. The MS spectrum of [¹⁴C]- and [¹³C] Compound I exhibited the (M+H)⁺, (¹³C−M+H)⁺, (M+NH₄)⁺, and (¹³C−M+NH₄)⁺ ions at m/z 846, 852, 863, and 869, respectively. A compound of R, 65.91 min exhibited the same molecular ions at m/z 846, 852, 863, and 869 as those of the [¹⁴C] Compound I standard, confirming this compound as the unchanged parent drug. A minor component with a R₁ of −66.2 min also yielded the (M+H)⁺, (¹³C−M+H)⁺, (M+NH₄)⁺, and (¹³C−M+NH₄)⁺ ions at m/z 846, 852, 863, and 869, respectively, indicating that this compound (M27) could be the 7-epi-isomer of [¹⁴C] Compound I. The epimerization of Taxol at 7-position has been reported. Since this isomer was not found in the [¹⁴C] Compound I stock solution used to spike the incubation mixture, it probably was a degradation product formed in a trace amount during incubation.

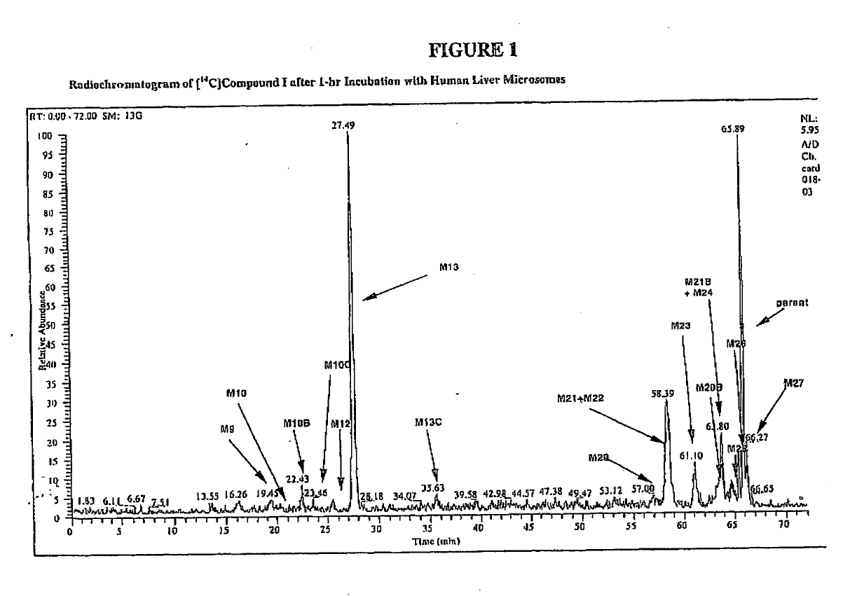

FIG. 1 shows the radiochromatogram of [¹⁴C] Compound I after 1 hr incubation with HLM. More than 10 radioactive components other than the parent drug were detected. The radioactive component with a R₁ of 65.89 min was confrimed by LC/RAM/(+)ESI-MS and MS/MS as the parent drug. A minor radioactive component at R₁ 66.27 min was identified by LC/RAM/(+)ESI-MS and MS/MS as the 7-epi isomer (M27). The same compound was also detected in the negative control incubation with human liver microsomes in a similar quantity and, therefore, was considered as a degradation product of [¹⁴C] Compound I rather than a metabolite.

Metabolite M13

M13 (R₁=27.49 min) was a major metabolite of [¹⁴C] Compound I formed by HLM. It exhibited (M+NH₄)⁺ and (¹³C−M+NH₄)⁺ ions at m/z 620 and 626, respectively, indicating a molecular weight of 602 daltons which was 243 amu lower than that of Compound I (MW 845). The characteristic ions at m/z 525, 345, 327 and 105 suggested that the compound had a structure that was similar to that of the baccatin moiety of Compound 1. Based on the data, M13 was proposed as a hydrolytic product of Compound I resulting from a loss of the side chain. M13 was also identified as a major metabolite in bile and urine following an oral dose of [¹⁴C] Compound I to rats. The proposed structure of M13 is shown below:

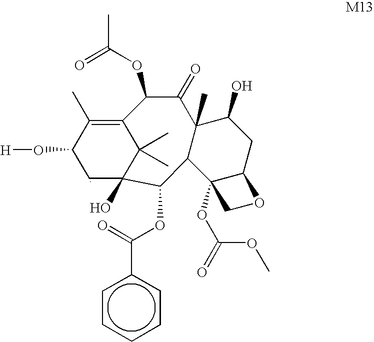

Metabolite M9

M9 (R₁=19.45 min) exhibited the (M+NH₄)⁺ and (¹³C−M+NH₄)⁺ ions at m/z 622 and 628, respectively, indicating a molecular weight of 604 daltons which was 2 amu higher than that of M13 (MW 602) suggesting the addition of two hydrogen atoms. The characteristic ion at m/z 527 resulted from the loss of a NH₃, H₂O and CH₃COOH from the ion at m/z 622. The ion at m/z 405 resulted from the loss of a benzoic acid moiety from the ion at m/z 527. The ion at m/z 465 resulted from the loss of NH₃, H₂O, and C₆H₅COOH from the ion at m/z 622. The ion at m/z 389, which was not a fragment ion of M13, probably resulted from the loss of NH₃, H₂O, C₆H₅COOH, and HCO₃CH₃ from the ion at m/z 622. The ion at m/z 369 probably resulted from the loss of a NH₃, CH₃COOH, and C₆H₅COOH group and 3 H₂O from the ion at m/z 622. Based on this information and the fact that this metabolite was more polar than M13 on HPLC, M9 was proposed as a metabolite derived from M13 with the C₉-keto group being reduced. The structure of M9 was proposed as follows:

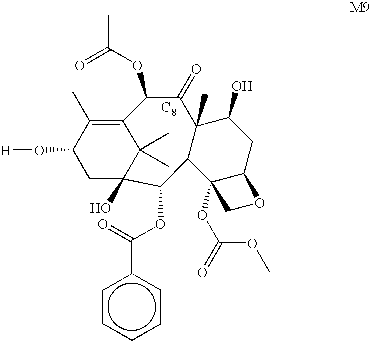

Metabolite M10

M10 (R₁=20.25 min) exhibited the (M+NH₄)⁺ and (¹³C−M+NH₄)⁺ ions at m/z 636 and 642, respectively, indicating a molecular weight of 618 daltons which was 16 amu higher than that of M13 (MW 602) suggesting the addition of one oxygen. In comparison to the fragmentation pattern of M13, the prominent ions at m/z 343 and 541 were 16 amu higher than the corresponding ions at m/z 327 and 525 of M13. The ion at m/z 327 could have resulted from the loss of NH₃, H₂O, CH₃COOH, HCO₃CH₃, C₆H₅COOH, and CH₄ from the ion at m/z 636. In addition, the ion at m/z 105 indicated that the phenyl ring was intact. Based on this information, the structure of M10 was proposed as follows:

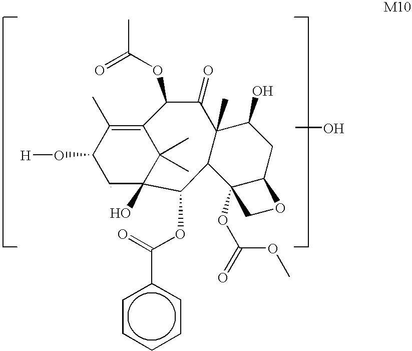

Note: the stereochemistry of the 7-position was left uncertain because the metabolite could be a 7-epimer of M10B, M10D, or M12, which were all characterized as monohydroxylated metabolites of the baccatin moiety.

Metabolite M10B

M10B (R₁=22.43 min) also exhibited the (M+NH₄)⁺ and (¹³C−M+NH₄)⁺ ions at m/z 636 and 642, respectively, indicating a molecular weight of 618 daltons which was 16 amu higher than that of M13 (MW 602) suggesting the addition of one oxygen. The ion at m/z 105 indicated that the phenyl ring was intact. M10B and M10 had the same molecular weight, but their MS/MS spectra and R₁'s were different. Thus. M10B was proposed as a monohydroxy-M13 with the site of hydroxylation undetermined.

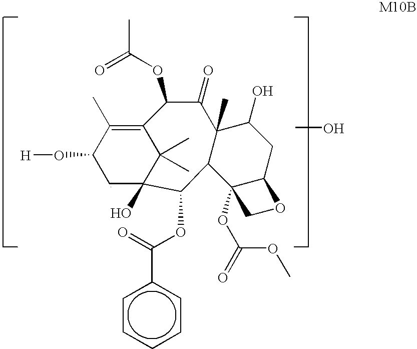

Metabolite M10C

M10C (R₁=23.46 min) also exhibited the (M+NH₄)⁺ and (¹³C−M+NH₄)⁺ ions at m/z 636 and 642, respectively, indicating a molecular weight of 618 daltons which was 16 amu higher than that of M13 (MW 602) suggesting the addition of one oxygen. In comparison to the fragmentation pattern of M13, the same characteristic ions at m/z 449, 345, and 327 of M13 were found. In addition, no distinctive ion resulting from the loss of CH₃COOH was detected. Instead, an ion at m/z 525 was detected. This ion resulted from the loss of HOCH₂COOH, indicating that oxidation probably occurred on the ester side chains instead of the baccatin ring. The structure of M10C was tentatively proposed as follows:

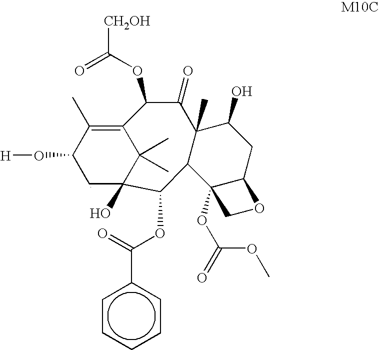

Metabolite M12

M12 (R₁=25.52 min) also exhibited the (M+NH₄)⁺ and (¹³C−M+NH₄)⁺ ions at m/z 636 and 642, respectively indicating a molecular weight of 618 daltons suggesting the addition of one oxygen. The MS/MS spectrum shows the characteristic ions at m/z 343, 361, and 541 that were 16 amu higher than the corresponding ions at m/z 327, 345 and 525 of M13 indicating that the benzoyl, acetyloxy, and methoxycarbonyloxy groups were intact. The characteristic ion at m/z 105 showed that the phenyl group was intact. The fragment ions at m/z 325 and 523 were derived from the loss of a water molecule from the ions at m/z 343 and 541, indicating the presence of an additional hydroxyl group on the baccatin ring. The fragment ions at m/z 325 and 523 were not detected in the MS/MS spectra of M10, M10B, and M10C. However, the exact position of hydroxylation has not been identified. M12 was also a metabolite identified in rat bile. The structure of M12 was proposed as follows:

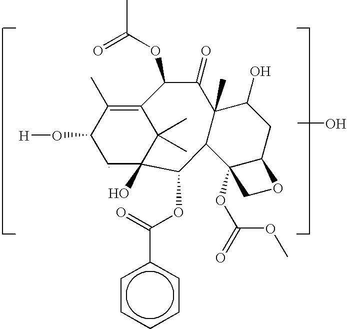

M12

Metabolite M13C

M13C (R₁=35.56 min) exhibited the (M+NH₄)⁺ and (¹³C−M+NH₄)⁺ ions at m/z 620 and 626, respectively, indicating a molecular weight of 602 daltons which was 243 amu lower than that of Compound I (MW 845). The MS/MS spectrum of M13C shows the same characteristic ions at m/z 105, 327, 345 and 525 of M13, suggesting that the compound was an isomer of M13. Based on the literature, M13C could be tentatively identified as the 7-epi-isomer of M13 and its proposed structure shown as follows:

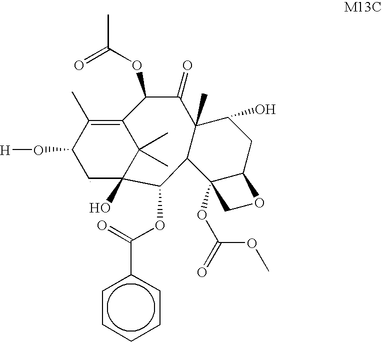

Metabolite M20

M20 (R₁=56.80 min) exhibited the (M+NH₄)⁺ and (¹³C−M+NH₄)⁺ ions at m/z 877 and 883 amu, respectively, indicating a molecular weight of 859 daltons which was 14 amu higher than that of the parent compound suggesting the addition of one oxygen and the loss of two hydrogens. The characteristic ions at m/z 585, 525, 449, 387, and 309 suggested that the baccatin ring moiety was intact. M20 was identified as a cyclization product in bile by MS/MS analysis and by analogy to the literature. Since there was no distinctive fragment showing where the cyclization occurred, the structure for M20 was proposed as follows:

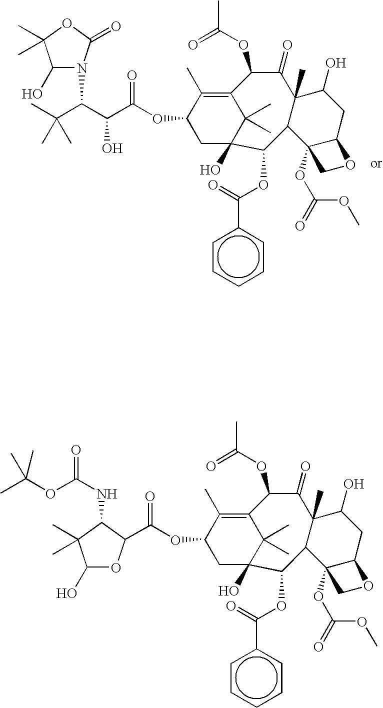

Metabolite M21

M21 (R₁=58.41 min) exhibited the (M+NH₄)⁺ and (¹³C−M+NH₄)⁺ ions at m/z 875 and 881 amu, respectively, indicating a molecular weight of 857 daltons which was 12 amu higher than that of the parent compound suggesting the addition of one oxygen and the loss of four hydrogens. The characteristic ions at m/z 585, 525, 449, 387, 327, and 309, that were also the characteristic ions of the parent drug, suggested that the baccatin moiety was intact. M21 was also detected in rat bile and proposed as a ring-closure metabolte of [¹⁴C] Compound I by analogy to the literature. The structure of M21 was proposed as one of the following.

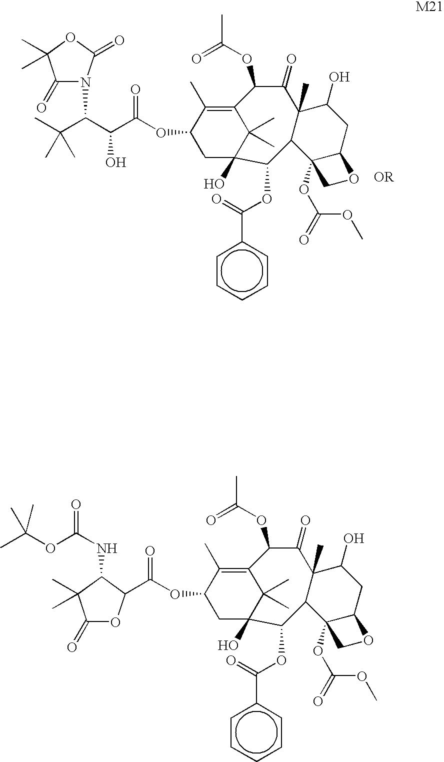

Metabolite M22

M22 (R₁=58.44 min) exhibited the (M+NH₄)⁺ and (¹³C−M+NH₄)⁺ ions at m/z 893 and 899 amu, respectively, indicating a molecular weight of 875 daltons which was 30 amu higher than that of the parent compound and suggesting the addition of two oxygens and the loss of two hydrogens. The characteristic ions at m/z 585, 525, 449, 387, and 327, that were also the characteristic ions of the parent drug, suggested that the baccatin moiety was intact. The characteristic ions at m/z 192, 236 and 292 were 30 amu higher than the corresponding ions at m/z 162, 206, and 262 of the parent drug indicating that one of the methyl groups of the t-butyl moiety was oxidized to the corresponding carboxylic acid. The prominent ions at m/z 236 and 192 resulting from the loss of C₄H₈ from the ion at m/z 292 and from the further loss of CO₂ indicated that both oxidations occurred on one of the methyl groups of the t-butyl moiety on C_{3′}. M22 was also identified in rat bile and its structure was proposed as follows:

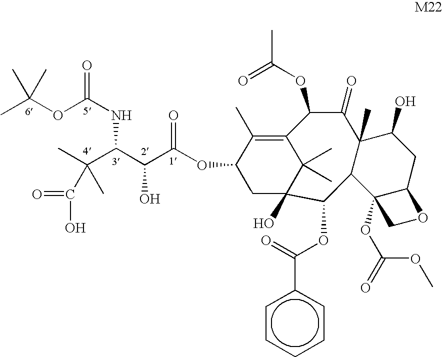

Metabolite M23

M23 (R₁=61.03 min) exhibited the (M+NH₄)⁺ and (¹³C−M+NH₄)⁺ ions at m/z 879 and 885 amu, respectively, indicating a molecular weight of 861 daltons which was 16 amu higher than that of the parent compound. The characteristic ions at m/z 567 and 525 indicated that the baccatin ring was intact. The characteristic ion at m/z 278 was 16 amu higher than the corresponding side-chain fragment ion at m/z 262 of the parent drug, indicating oxidation of the side chain. M23 was identified as a metabolite of [¹⁴C]Compound I in rat bile and its structure was proposed as follows:

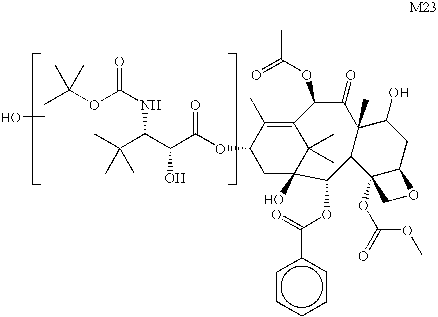

Metabolite M20B

M20B (R₁=−63.7 min) exhibited the (M+NH₄)⁺ and (¹³C−M+NH₄)⁺ ions at m/z 877 and 883 amu, respectively, indicating a molecular weight of 859 daltons which was 14 amu higher than that of the parent compound suggesting the addition of one oxygen and the loss of two hydrogens. The charatcristic ions at m/z 585, 525, 449, 405, 387, and 309 suggested that the baccatin ring moiety was intact. Based on the data and by analogy to the literature, M20B was proposed as a metabolite derived from oxidation of the side chain followed by ring closure of the oxidative product. The structure of M20B was proposed as one of the following:

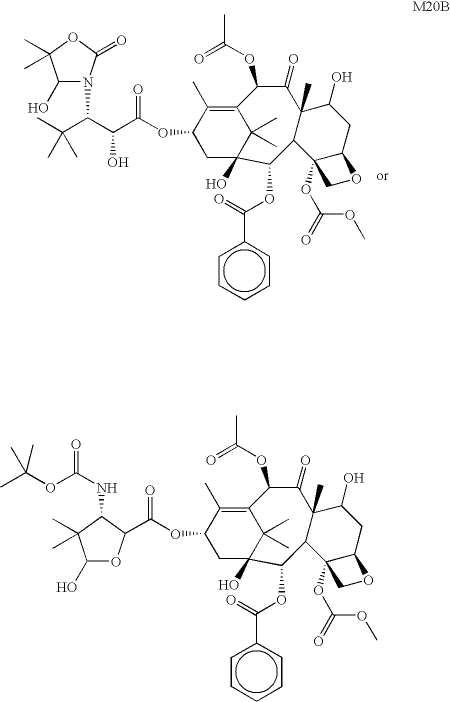

Metabolite M21B

M21B (R₁=63.75 min) exhibited the (M+NH₄)⁺ and (¹³C−M+NH₄)⁺ ions at m/z 875 and 881 amu, respectively, indicating a molecular weight of 857 daltons which was 12 amu higher than that of the parent compound and suggesting the addition of one oxygen and a loss of four hydrogens. The LC/MS/MS analysis of M21B from the incubation of [¹⁴C]Compound I with human and monkey liver microsomes showed the characteristic ions at m/z 585, 525, 449, 345, 327, and 309, indicating that the baccatin ring was intact. Based on these data and by analogy to the literature, the structure of M21B was proposed as one of the following:

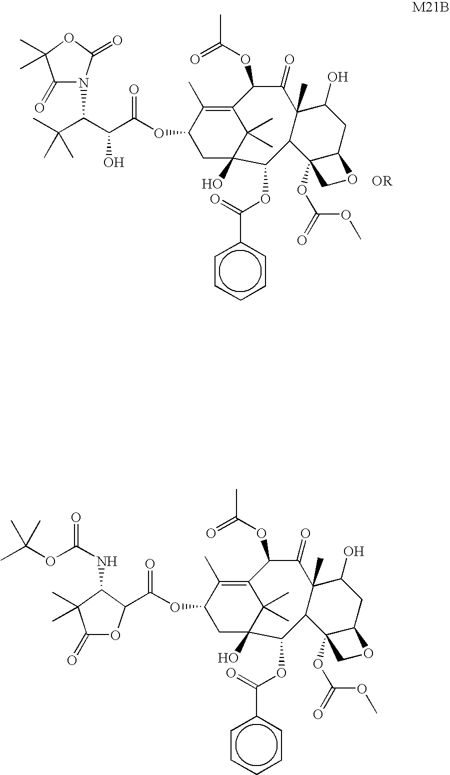

Metabolite M24

M24 (R₁=63.80 min) exhibited the (M+NH₄)⁺ and (¹³C−M+NH₄)⁺ ions at m/z 893 and 899 amu, respectively, indicating a molecuiar height of 875 daltons which was 30 amu higher than that of the parent compound and suggesting the addition of two oxygens and a loss of two hydrogens. The characteristic ions at m/z 525, 567, and 585 indicated that the baccatin ring was intact. Based on the data and since there was no distinctive ion showing on which t-butyl group the oxidation occurred, the structure of M24 was proposed as one of the following: 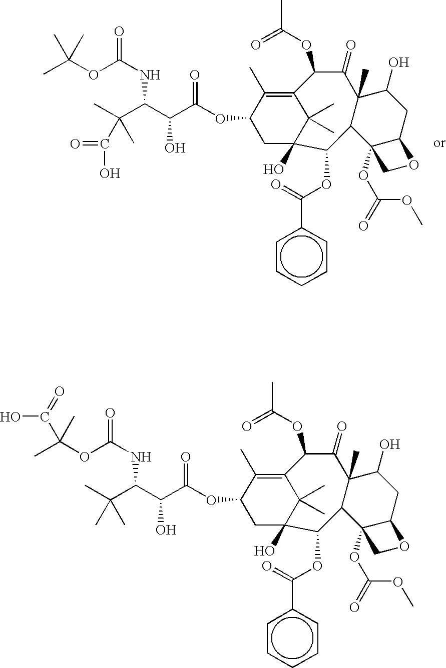
The possibility that M24 could be a 7-epi isomer of M22 cannot be also excluded.

Metabolite M25

M25 (R₁=64.63 min) exhibited the (M+NH₄)⁺ and (¹³C−M+NH₄)⁺ ions at m/z 907 and 913 amu, respectively, indicating a molecular weight of 879 daltons which was 44 amu higher than that of the parent compound and suggesting the addition of three oxygens and the loss of four hydrogens. The presence of characteristic ions at m/z 585, 525, 449, 403, 387, and 327 indicated that the baccatin ring was intact. The characteristic ion at m/z 306 was 44 amu higher than the corresponding ion at m/z 262 of the parent drug, confirming that 44 amu was added to the side chain. The characteristic ions at m/z 250 and 206 of M25 were derived from the loss of t-butyl moiety and CO₂, respectively. These characteristic ions at m/z 306, 250, and 205 indicated that the cyclization occurred on the t-butyl group on C_{3·}. Based on the information, the structure M25 was proposed as follows:

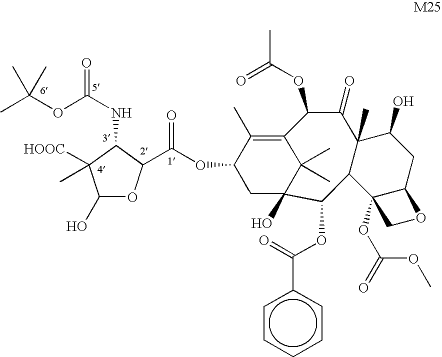

Metabolite M26

M26 (R₁=65.44 min) exhibited the (M+NH₄)⁺ and (¹³C−M+NH₄)⁺ ions at m/z 821 and 827 amu, respectively, indicating a molecular weight of 803 daltons which was 42 amu lower than that of the parent compound suggesting the loss of an acetyl group. MS/MS data of M26 in the human microsome incubate did not provide any conclusive fragment ions. MS/MS data was, therefore, obtained from the rat microsome incubate. The MS/MS spectrum of M26 from RLM incubation yielded fragment ions at m/z 262, and 206, indicating that the side chain was intact. The ion at m/z 543 was 42 amu less than the corresponding ion at m/z 585 found of the parent drug. Based on this information, the structure M26 was proposed as follows:

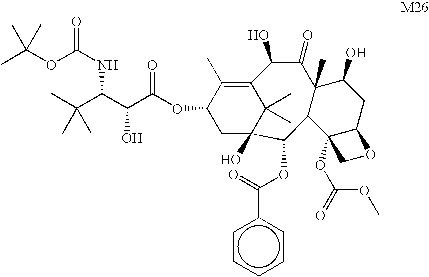

M26 was also found in the negative control samples in which [¹⁴C] Compound I was incubated with liver microsomes from five species in the absence of NADPH or with heat-inactivated microsomes. M26 was considered as a degradation product of [¹⁴C] Compound I in the incubation system.

2. Metabolites Identified from Monkey Liver Microsome Incubation

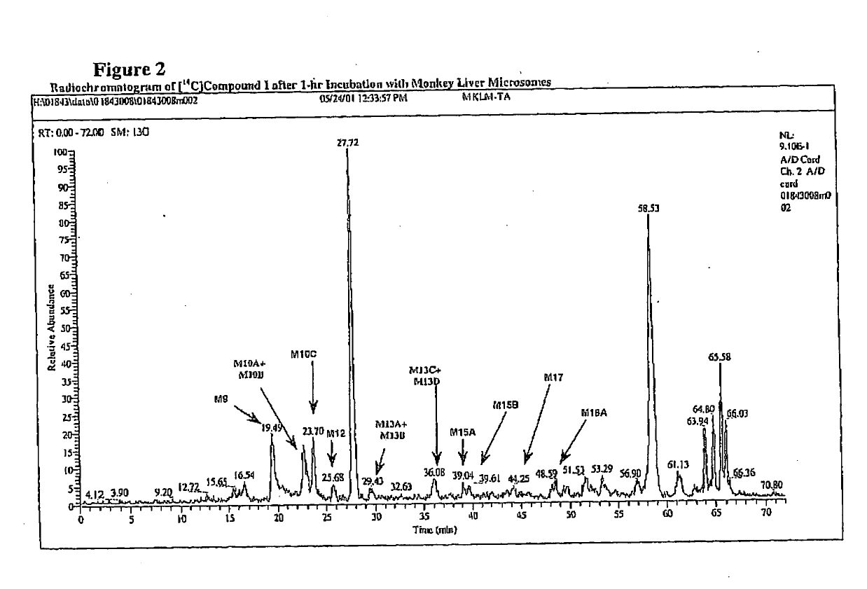

The metabolite profile of [¹⁴C] Compound I in monkey liver microsomes was the most complex one among the five species investigated. FIG. 2 shows the radiochromatogram of [¹⁴C] Compound I after 1 hr incubation with monkey liver microsomes. A total of 30 compounds, other than the parent drug, were characterized. The identities of M9, M10B, M10C, M12, M13, M13C, M20, M21, M21B, M22, M23, M24, M25, M26, parent, and M27 that were found in HLM incubation were confirmed by LC/RAM(+)ESI/MS and/or MS/MS.

The following shows the identification or characterization of metabolites in MKLM incubation that were not found in HLM incubation.

Metabolite M10A

M10A (R₁=22.68 min) exhibited the (M+NH₄)⁺ and (¹³C−M+NH₄)⁺ ions at m/z 578 and 584, respectively, indicating a molecular weight of 560 daltons which was 42 amu lower than that of M13 (MW 602) suggesting the loss of an acetyl group. The characteristic ions at m/z 543, 525, 449, 467, 345, and 327 indicated that both methoxycarbonyloxy- and benzoyl moieties were intact. Thus, the structure of M10A was proposed as follows:

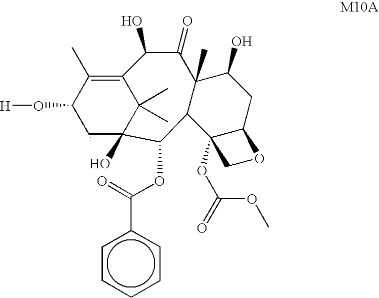

Metaboalte M13A

M13A (R₁=29.47 min) exhibited the (M+NH₄)⁺ and (¹³C−M+NH₄)⁺ ions at m/z 576 and 582, respectively, indicating a molecular weight of 558 daltons which was 44 amu lower than that of M13 (MW 602) suggesting the loss of an acetyl group and two hydrogens. In comparison to the fragmentation pattern of M13, the absence of fragment ions at m/z 525 and 543, which resulted from the loss of acetic acid from M13, indicated the absence of acetyloxy moiety in the molecule. The ion at m/z 361 resulted from the loss of NH₃, HCO₃CH₃, and C₆H₅COOH from the ion at m/z 576. A further loss of a water molecule from the ion at m/z 361 yielded the ion at m/z 343. The data indicated that both the methoxycarbonyloxy and benzoyl moieties were intact. Although the MS data were not sufficient enough to determine which hydroxyl group between the two (on C₇ and C₁₃) had been oxidized, the hydroxyl group on C₁₃ was considered to be the more preferred site for oxidation based on two factors. These were less steric hinderance for the reaction and its greater acidic nature due to the presence of an adjacent carbon-carbon double bond. Steric hinderance and the acidity of alcohols were shown to play important roles for the rate of oxidation. The structure of M13A was proposed as follows:

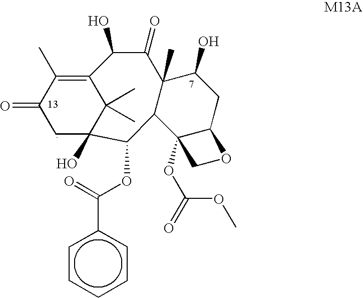

Metabolite M13B

M13B (R₁=29.72 min) exhibited the (M+NH₄)⁺ and (¹³C−M+NH₄)⁺ ions at m/z 578 and 584, respectively, indicating a molecular weight of 560 daltons which was 42 amu lower than that of M13 (MW 602). The fragment ion at m/z 543 resulted from the loss of NH₃ and H₂O. In comparison to the fragmentation pattern of M13, the characteristic ions at m/z 361 and 483 were probably derived from the loss of both benzoic acid and acetic acid and acetic acid alone, respectively, indicating that both of these moieties were intact. The absence of a fragment ion resulted from the loss of CH₃OCOOH indicated that the molecule did not contain this functional group. In addition, the molecular weight of the metabolite indicated an addition of an oxygen to the resulting hydrolysis product. Thus, the structure of M13B was proposed as follows:

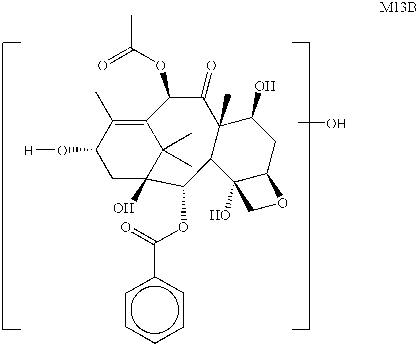

Metabolite 13D

M13D (R₁=36.09 min) exhibited the (M+NH₄)⁺ and (¹³C−M+NH₄)⁺ ions at m/z 618 and 624, respectively, indicating a molecular weight of 600 daltons which was 2 amu lower than that of M13 (MW 602) indicating the loss of two hydrogens. In comparison to the fragmentation pattern of M13, the characteristic ions at m/z 325, 343, 403, 465, and 541 were all 2 amu lower than the corresponding ions of M13, suggesting that dehydrogenation occurred from one of the hydroxyl groups in M13. Although the MS data were not sufficient enough to determine which hydroxyl group between the two (on C₇ and C₁₃) had been oxidized, the hydroxyl group on C₁₃ was considered to be the more preferred site for oxidation based on two factors. These were less steric hinderance for the reaction and its greater acidic nature due to the presence of an adjacent carbon—carbon double bond. Steric hindrance and the acidity of alcohols were shown to play important roles for the rate of oxidation. Based on this information, the structure of M13D was proposed as follows:

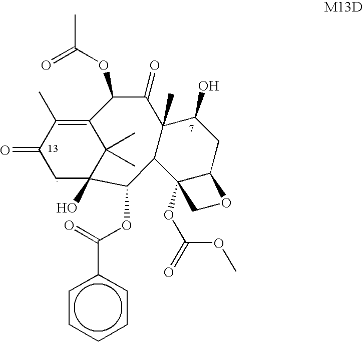

Metabolite M15A

M15A (R₁=39.11 min) exhibited the (M+NH₄)⁺ and (¹³C−M+NH₄)⁺ ions at m/z 891 and 897, respectively, indicating a molecular weight of 873 daltons which was 28 amu higher than that of Compound I (MW 845) suggesting the addition of two oxygens and the loss of four hydrogens. The characteristic ions at m/z 585, 567, 369, 327, and 105 indicated that the baccatin ring was intact. Based on the information, M15A was proposed as a metabolite derived from oxidation of the side chain followed by ring closure of the oxidative product and dehydrogenation. Since there was no distinctive ion showing where the cyclization occurred, the structure of M15A was proposed as one of the following:

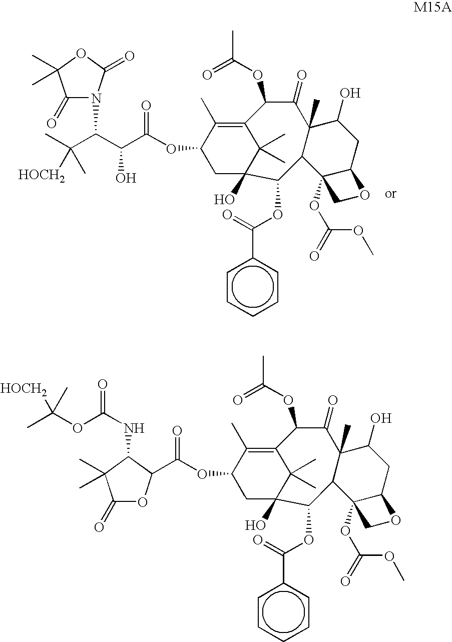

Metabolite M15B

M15B (R₁=39.64 min) exhibited the (M+NH₄)⁺ and (¹³C−M+NH₄)⁺ ions at m/z 909 and 915, respectively, indicating a molecular weight of 891 daltons which was 46 amu higher than that of Compound I (MW 845) suggesting the addition of three oxygens and the loss of two hydrogens. The MS/MS spectrum exhibits the characteristic ions at m/z 567 and 327 indicating that the baccatin ring was intact. The fragment ion at m/z 308 was 46 amu higher than the corresponding ion at m/z 262 of Compound I, indicating that the reactions occurred on the side chain. Based on the limited MS/MS data, the structure of M15B was proposed as follows:

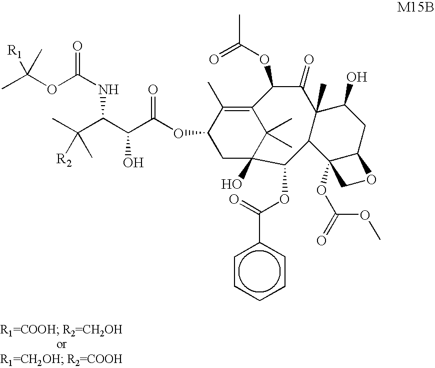

Metabolite M17

M17 (R₁=44.25 min) exhibited the (M+NH₄)⁺ and (M+H)⁺ ions at m/z 879 and 885 amu, respectively, indicating a molecular weight of 861 daltons which was 16 amu higher than the parent compound suggesting the addition of one oxygen. The characteristic ions at m/z 585, 567, 509, and m/z 403 suggested that the baccatin moiety was intact. The characteristic ions at m/z 278, 222, and 178 was 16 amu higher than the corresponding ions at m/z 262, 206, and 162 of the parent componnd, suggesting that an oxygen was added to the t-butyl group on the side chain. The structure of M17 was proposed as follows:

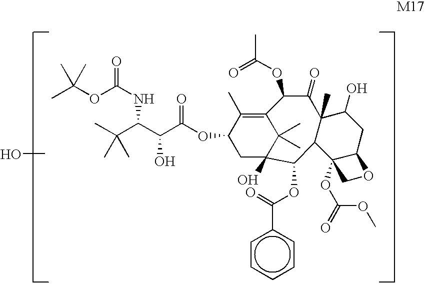

Metabolite M18A

M18A (R₁=48.50 min) exhibited the (M+NH₄)⁺ and (¹³C−M+NH₄)⁺ ions at m/z 835 and 841, respectively, indicating a molecular weight of 817 daltons which was 28 amu lower than that of Compound I (MW 845). The characteristic ions at m/z 525, 449, 431, and 327 indicated the loss of an acetyl group (42 amu) from the baccatin ring and the addition of 14 amu to the side chain. The ion at m/z 258 was believed to have derived from the side chain after the loss of a H₂O molecule. Based on the data, the structure of M18A was proposed as one of the following:

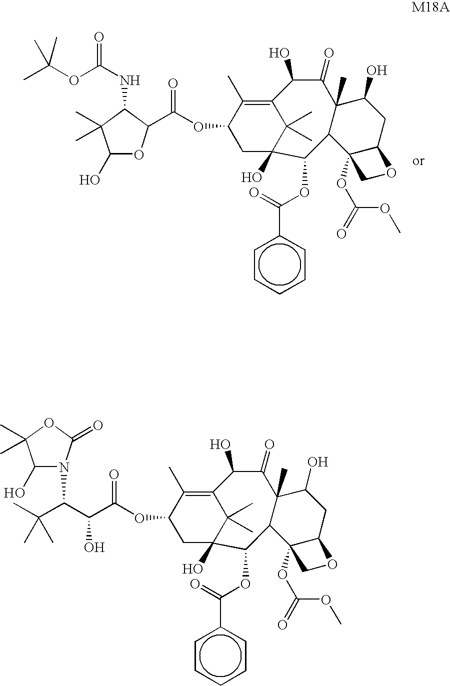

Metabolite M18B

M18B (R₁=49.66 min) exhibited the (M+NH₄)⁺ and (¹³C−M+NH₄)⁺ ions at m/z 851 and 857, respectively, indicating a molecular weight of 833 daltons which was 12 amu lower than that of the parent. The characteristic ions at m/z 292, 236 and 192 indicated the addition of 30 amu to the side chain. The ions at m/z 236 and 192 were derived from the loss of t-butyl moiety and CO₂, respectively, indicating that oxidation occurred on the t-butyl group on C_{3′}. The characteristic ion at m/z 543 suggested the loss of an acetyl group (42 amu) from the baccatin ring. The structure of M18B was proposed as follows:

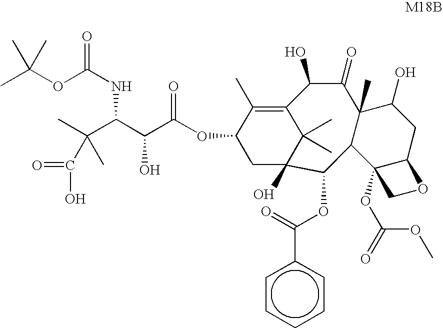

Metabolite M18C

M18C (R₁=51.52 min) exhibited the (M+NH₄)⁺ and (¹³C−M+NH₄)⁺ ions at m/z 833 and 839, respectively, indicating a molecular weight of 815 daltons which was 30 amu lower than that of the parent drug. The characteristic ion at m/z 543 suggested the loss of an acetyl group (42 amu) from the baccatin ring after the loss of the side chain. The ions at m/z 449, 345, and 327 also supported that deacetylation occurred. Therefore, M18C was most likely derived from the combination of deacetylation, addition of an oxygen and loss of four hydrogens from Compound I. Based on the data, the structure of M18C was proposed as one of the following:

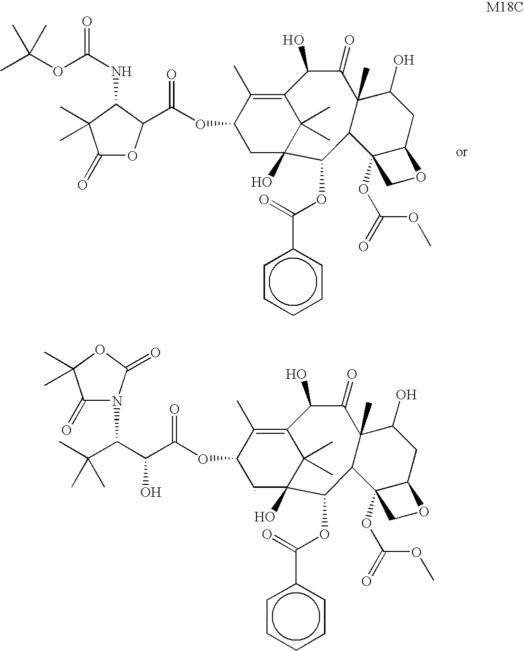

Metaboilte M19

M19 (R₁=52.43 min) exhibited the (M+NH₄)⁺ and (¹³C−M+NH₄)⁺ ions at m/z 879 and 885, respectively, indicating a molecular weight of 861 daltons which was 16 amu higher than that of the parent drug. The characteristic ions at m/z 585, 525, 449, 327, 369, and 387 suggested that the baccatin ring was intact, indicating the addition of one oxygen to the side chain. It could be a 7-epi isomer of M17. Thus, the structure M19 was proposed as follows:

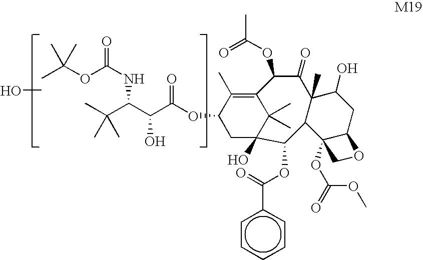

Metabolite M19A

M19A (R₁=53.28 min) exhibited the (M+NH₄)⁺ and (¹³C−M+NH₄)⁺ ions at m/z 851 and 857, respectively, indicating a molecular weight of 833 daltons which was 12 amu lower than that of the parent drug. The characteristic ion at m/z 543 suggested the loss of an acetyl group and the ions at m/z 327, 345, 403, and 525 indicated that the remaining structure of the baccatin ring was intact. The characteristic ion at m/z 292 indicated the addition of 30 amu to the side chain suggesting the addition of two oxygens and the loss of two hydrogens. Based on the fact that no distinctive peaks, such as ions at m/z 236 and 192, were found from the loss of t-butyl and CO₂ as detected in the spectra of M18B, the structure of M19A was proposed as follows:

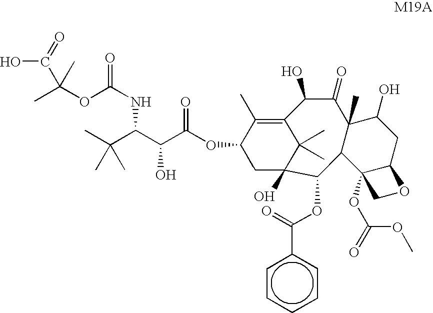

Metabolite M19B

M19B (R₁=53.51 min) exhibited the (M+NH₄)⁺ and (¹³C−M+NH₄)⁺ ions at m/z 877 and 883, respectively, indicating a molecular weight of 859 daltons which was 14 amu higher than that of the parent drug. Due to insufficient information from the MS/MS analysis, the structure of the compound was not proposed.

Metabolite M23D

M23D (R₁=65.56 min) exhibited the (M+NH₄)⁺ and (¹³C−M+NH₄)⁺ ions at m/z 879 and 886, respectively, indicating a molecular weight of 861 daltons which was 16 amu higher than that of the parent suggesting the addition of one oxygen. The characteristic ions at m/z 162 and 206 indicated that the side chain was intact suggesting that the oxygen was added to the baccatin ring. The structure M23D was proposed as follows: 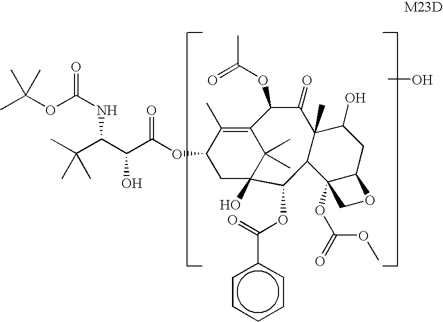
3. Metabolites Identified from Rat Liver Microsome Incubation

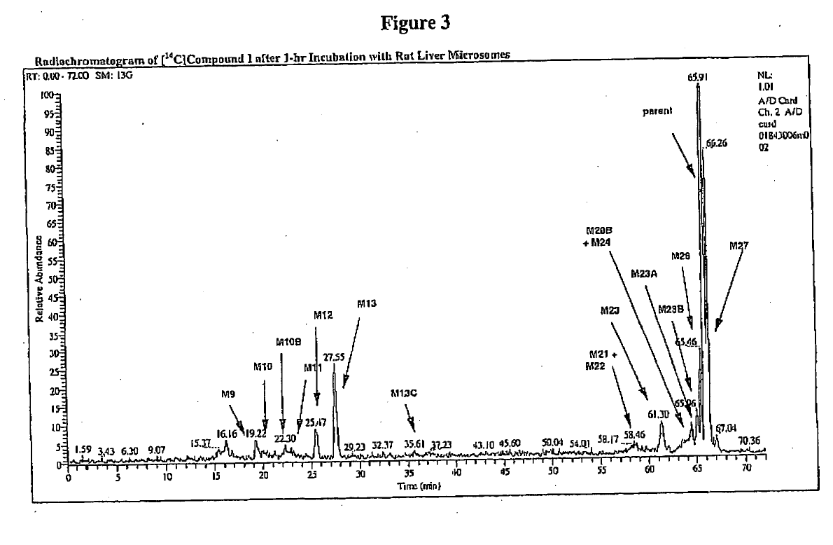

FIG. 3 shows the radiochromatogram of [¹⁴C] Compound I after 1 hr incubation with rat liver microsomes. A total of 16 compounds other than the parent drug were characterized. Most of these compounds including M9, M10, M10B, M12, M13, M13C, M20B, M21, M22. M23, M24, M26, parent, and M27 that were also found in HLM and/or MKLM incubation, were confirmed by LC/RAM/(+)ESI/MS and/or MS/MS.

The following shows the identification or characterization of three metabolites in RLM incubation that were not found in HLM and/or MKLM incubations.

Metabolite M11

M11 (R₁=23.10 min), which was found only in RLM, was also identified in rat bile. M11 exhibited (M+NH₄) and (¹³C−M+NH₄)⁺ ions at m/z 650 and m/z 656, indicating a molecular weight of 632 daltons which was 30 amu higher than that of M13 (MW 602) and suggesting the addition of two oxygens and the loss of two hydrogens. In comparison to the fragmentation pattern of M13, the prominent ions at m/z 479, 435, and 357 were all 30 amu higher than the corresponding fragment ions at m/z 449, 405, and 327, respectively, of M13. These data indicated that the benzoyl, acetyloxy, and methoxycarbonyloxy groups were all intact. Thus, the two oxygens were most likely added to the methyl group at the C₁₂ position. Based on these data, the structure of M11 was proposed as follows:

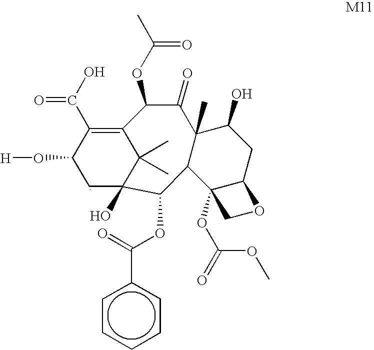

Metabolite M23A

M23A (R₁=64.35 min) exhibited the (M+NH₄)⁺ and (¹³C−M+NH₄)⁺ ions at m/z 879 and 885, respectively, indicating a molecular weight of 861 daltons which was 16 amu higher than the parent compound (MW 845) suggesting the addition of an oxygen. MS/MS analysis of this compound was not performed due to the small amount of material.

Metabolite M23B

M23B (R₁=65.03 min) exhibited the (M+NH₄)⁺ and (¹³C−M+NH₄)⁺ ions at m/z 879 and 885, respectively, indicating a molecular weight of 861 daltons which was 16 amu higher than that of the parent compound suggesting the addition of one oxygen. The characteristic ions at m/z 525 and 325 were 16 amu higher than the corresponding fragment ions of Compound I indicating that the oxygen was added to the baccatin ring. In addition, the ions at m/z 262 and 206 show that the side chain was unchanged. Based on the data, the structure M23B was proposed as follows: 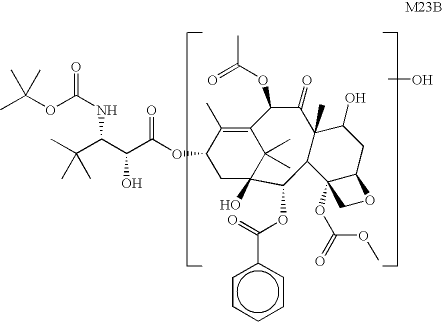
4. Metabolites Identified from Mouse Liver Microsome Incubation

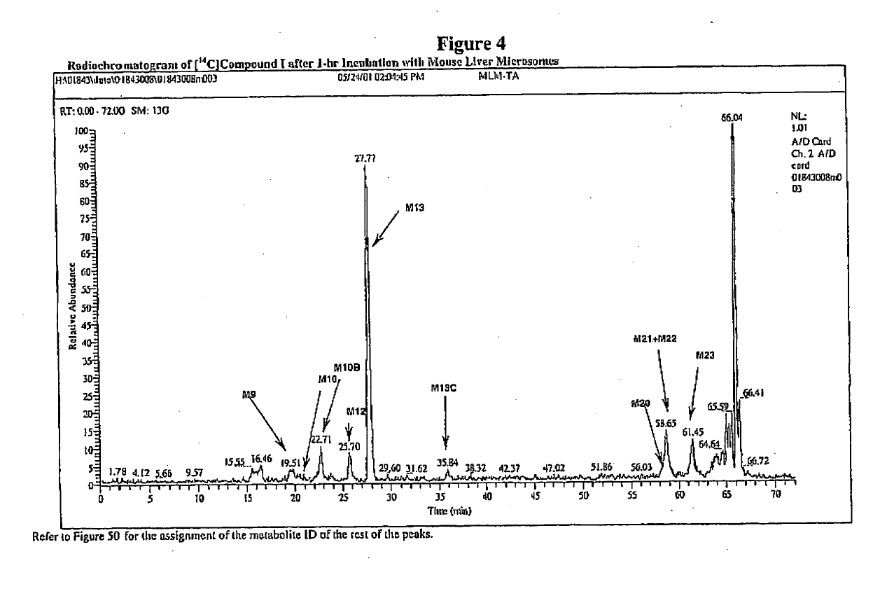

FIG. 4 shows the radiochromatograms of [¹⁴C] Compound I after 1 hr incubation with mouse liver microsomes. A total of 19 compounds other than the parent drug were detected. Most of these compounds including M9, M10, M10B, M12, M13, M13C, M20, M22, M23, M20B, M21B, M24, M23A, M23B, M26, parent and M27, that were also found in HLM and/or MKLM incubation, were confirmed by LC/RAM/(+)ESI/MS and/or MS/MS.

The following shows the identification or characterization of a metabolite in MLM incubation that was not found in the HLM, MKLM or RLM incubation.

Metabolite M23C

M23C (R₁=65.29 min) exhibited the (M+NH₄)⁺ and (¹³C−M+NH₄)⁺ ions at m/z 879 and 885, respectively, indicating a molecular weight of 861 daltons which was 16 amu higher than that of the parent compound suggesting the addition of one oxygen. Although further analysis by MS/MS was not conducted due to insufficient amount of compound, this compound was considered to have a similar structure as those of M23A, M23B, or M23D and the structure was proposed as follows: 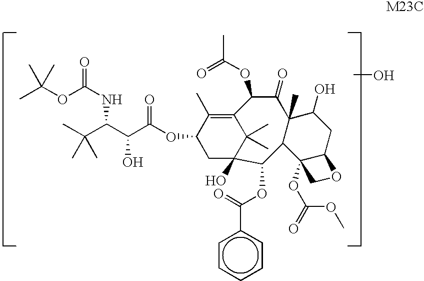
5. Metabolites Identified from Dog Liver Microsome Incubation

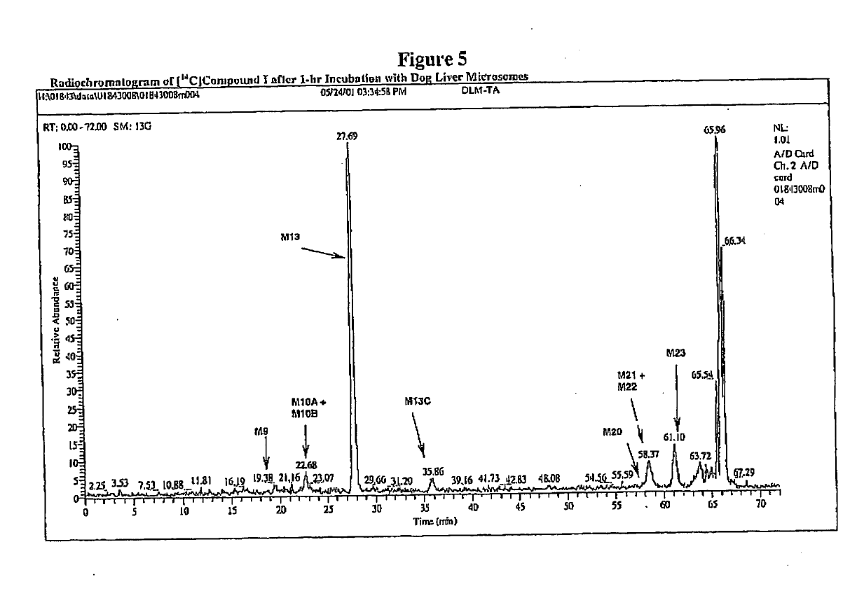

FIG. 5 shows the radiochromatograms of [¹⁴C] Compound I after 1 hr incubation with dog liver microsomes. A total of 19 compounds other than the parent drug were detected. All of these compounds including M9, M10A, M10B, M13, M13C, M20. M21, M22, M23, M20B. M21B, M24, M23A, M23B, M23C, M23D, M25, M26, parent and M27, which were also found in HLM, MKLM, RLM or MLM incubation, were confirmed by LC/RAM/(+)ESI/MS and/or MS/MS.

Metabolite Profiles in Mouse, Rat, Dog, Monkey, and Human Liver Microsomes

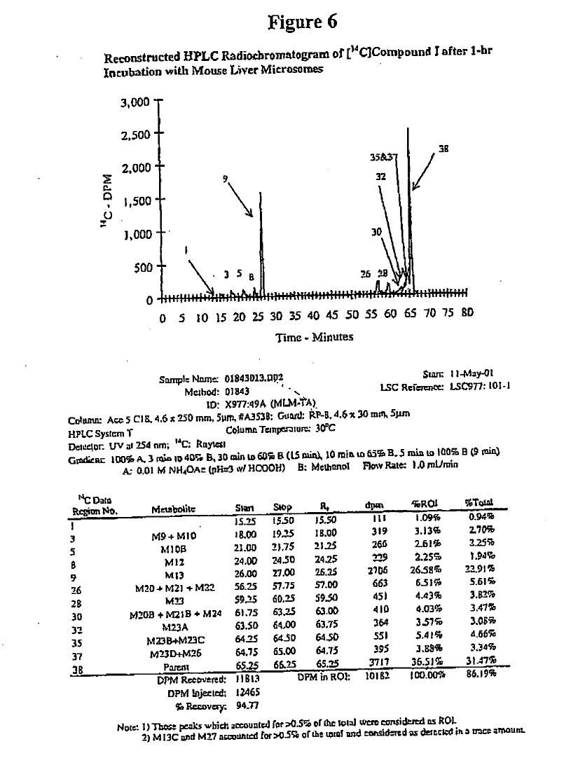

FIGS. 6 to 10 show the radiochromatograms generated by collecting and counting fractions of [¹⁴C] Compound I after 1 hr incubation with mouse, rat, dog, monkey and human liver microsomes.

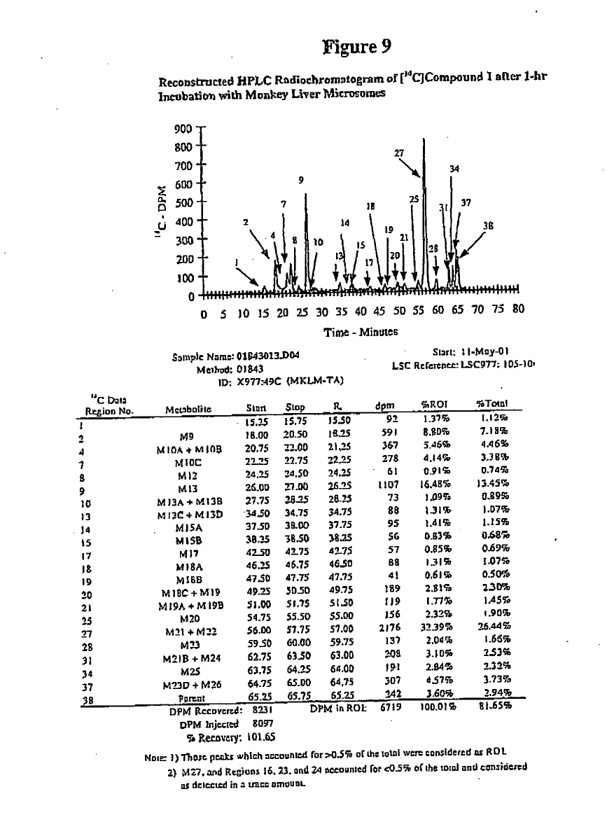

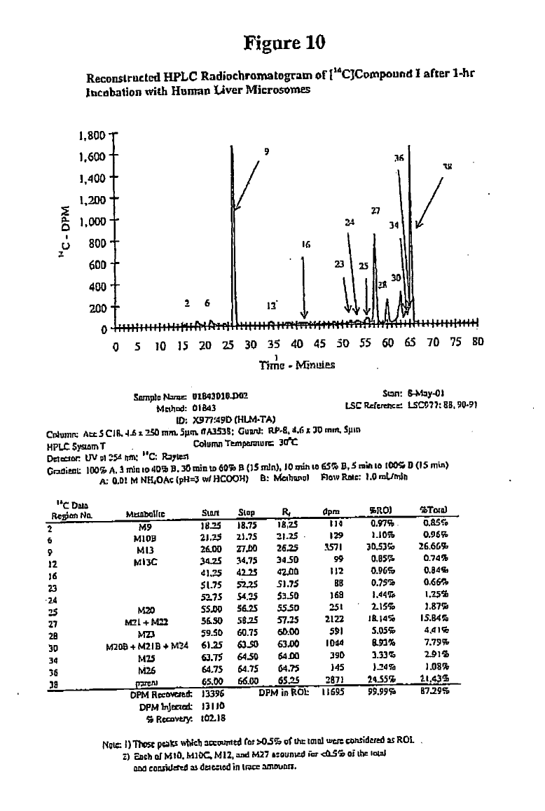

HLM metabolized [¹⁴C] Compound I to numerous metabolites, seventeen of them were identified or characterized by LC/RAM/(+)-ESI-MS and MS/MS (FIG. 10). Note that the number of metabolites detected as ROI in FIG. 10 were smaller than that identified or characterized by LC/MS because only those peaks accounting for >0.5% of the total radioactivity were considered as ROI. After 1 hr incubation, 21.4% of the [¹⁴C] Compound I in the incubation mixture was recovered unchanged. In general, the identified metabolites of [¹⁴C] Compound I in HLM could be classified into two categories: one derived from cleavage of the ester side chain at the C₁₃, position followed by oxidation of the formed baccatin moiety (Category I metabolites); and the other derived from direct oxidation of [¹⁴C] Compound I either on the side chain or on the baccatin moiety or both (Category II metabolites). A major metabolite (M13) in HLM was identified as a product derived from cleavage of the side chain. M13 alone accounted for 26.7% of the total radioactivity in incubation mixture. Category I metabolites (M9, M10B, M13 and M13C) together accounted for 29.2% of the total radioactivity in incubation mixture. Other Category I metabolites (M10, M10C, and M12), were detected in trace amounts by LC/MS analysis. Category II metabolites (M20-M25), mainly the metabolites derived from successive oxidations of the t-butyl ester group of the side chain followed by spontaneous cyclization of the putative aldehyde and acid derivatives, together accounted for 32.8% of the total radioactivity in incubation mixture. A small amount of a deacetylated product of Compound I (M26; −1%) and a trace amount of 7-epi-Compound I (M27; R₁−66 min) were identified both in the negative control incubations as well as in the HLM incubation. They were believed to be degradation products rather than metabolites. Other minor metabolites that accounted for the remaining radioactivity in the incubation were not identified due to small amounts of the material.

[¹⁴C]Compound I was extensively metabolized by MKLM. After 1 hr incubation, −3% of the parent drug in the incubation mixture remained unchanged. A total of 29 metabolites or degradation products in MKLM incubation were identified or characterized (FIGS. 2 and 9). The radiochromatogram (FIG. 9) shows four prominent metabolites (M9, M13, M21 and M22) which, together, accounted for 47.1% of the total radioactivity in incubation mixture. Most of the metabolites formed by MKLM were similar to those of HLM. Ten metabolites of Category I together accounted for 31.2% of the total radioactivity in incubation mixture. The corresponding value for 18 metabolites in Category II was 46.4%.

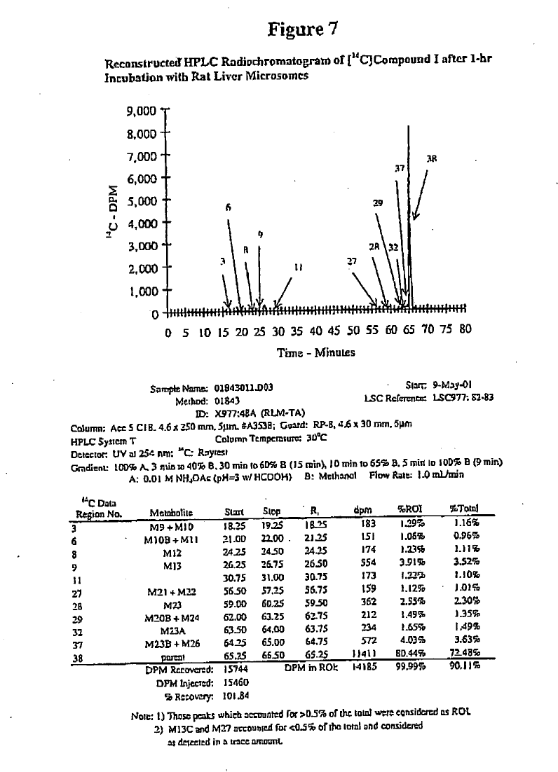

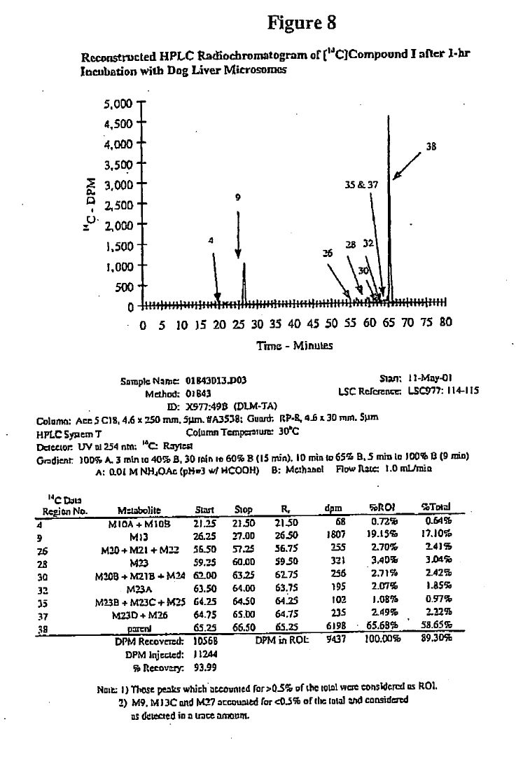

After 1 hr incubation of [¹⁴C] Compound I with DLM, 59.7% of the parent drug in the incubation mixture remained unchanged (FIGS. 5 and 8). A total of 19 metabolites or degradation products in the incubation mixture were identified or characterized. M13 was the most prominent metabolite formed by DLM. Alone it accounted for 17.1% of the total radioactivity in incubation mixture. The other identified metabolites were mainly the Category II metabolites.

[¹⁴C] Compound I was most stable in RLM among the five species tested. After 1 hr incubation, 72.5% of the parent drug remained unchanged (FIGS. 3 and 7). A total of 16 metabolites or degradation products in the incubation mixture were identified or characterized. No single prominent metabolites were observed. Six metabolites of Category I together accounted for 6.8% of the total radioactivity in the incubation mixture. The corresponding value for the 8 metabolites of Category II was 9.8%.

After 1 hr incubation of [¹⁴C] Compound I with MLM, 31.5% of the parent drug remained unchanged. Nineteen metabolites or degradation products in the incubation mixture were identified or characterized (FIGS. 4 and 6). M13 was the most prominent metabolite alone it accounted for 22.9% of the total radioactivity in incubation mixture. Five metabolites of Category I together accounted for 29.8% of the total radioactivity in incubation mixture. The corresponding value for the 13 metabolites of Category II was 24.0%.

### CONCLUSION

Metabolism of [¹⁴C] Compound I in mouse, rat, dog, monkey, and human lver microsomes was moderate to extensive. After 1 hr incubation of [¹⁴C] Compound I with mouse, rat, dog, monkey and human liver microsomes, 31.5%, 72.5%, 58.7%, 2.9% and 21.4% of the drug in the incubation mixtures remained unchanged. The rank order of metabolic stability of [¹⁴C] Compound I in the liver microsomes of the five species was; rat>dog>mouse>human>monkey.

[¹⁴C] Compound I was metabolized via the similar metabolic pathways among the five species, namely by hydrolysis, oxidation, ring-closure, reduction and dehydrogenation. In general, the identified metabolites could be classified into two categories: one derived from cleavage of the ester side chain at the C₁₃-position followed by oxidation of the resulting baccatin moiety (Category I metabolites); and the other derived from direct oxidation of [¹⁴C] Compound I either on the side chain or on the baccatin moiety or both (Category II metabolites), The Category I metabolites together accounted for 29.8%, 6.8%, 17.7%, 31.2% and 29.2%, respectively, of the total radioactivity in mouse, rat, dog, monkey and human incubations or 43%, 25%, 43%, 32% and 37% of the total formed metabolites. The total formed metabolites represent % total excluding the % total of parent. The corresponding values for the Category II metabolites were 24.0%, 9.8%, 12.9%, 46.4% and 32.8% or 35%, 36%, 31%, 48% and 42%, respectively. Thus, the liver microsomes of each of the five species formed about the same amounts of Categories I and II metabolites of [¹⁴C] Compound I.

In conclusion, all HLM metabolites were also detected in the four aninal species tested. These results indicate that the animals in the toxicology studies will be exposed to the same metabolites as in humans.

The following summarizes the mass spectrometric analysis of Compound I and its metabolites in mouse, rat, dog, monkey and human liver microsome incubations:

Summary of the Mass Spectrometric Analysis of Compound I and Its Metabolites
in Mouse, Rat, Dog, Monkey and Human Liver Microsome Incubations

| | | | TSQ | Structure Asignment | |
|---|---|---|---|---|---|
| Compound | R₁(min)¹ | R₁(min)² | Characteristic Ions (m/z) | MW (Da) | Source |
| M9 | −19.5 | 18.25-18.5 | 622 (M + NH₄)⁻ | 604 | HLM, MKLM, RLM |
| | | | 628 (¹³C − M + NH₄)⁻ | | MLM, DLM |
| M10 | −20.3 | −18.25 | 636 (M + NH₄)⁻ | 618 | HLM, RLM, MLM |
| | | | 642 (¹³C − M + NH₄)⁻ | | |
| M10A | −22.7 | 21.25-21.5 | 578 (M + NH₄)⁺ | 560 | MKLM, MLM, DLM |
| | | | 584 (¹³C − M + NH₄)⁺ | | |
| M11 | −23.1 | 21.25-21.5 | 650 (M + NH₄)⁺ | 632 | RLM |
| | | | 856 (¹³C − M + NH₄)⁻ | | |
| M10B | −22.4 | 21.25-21.5 | 635 (M + NH₄)⁺ | 618 | HLM, MKLM, DLM, |
| | | | 642 (¹³C − M + NH₄)⁻ | | RLM |
| M10C | −23.5 | 22.25 | 636 (M + NH₄)⁻ | 618 | HLM, MKLM |
| | | | 542 (¹³C − M + NH₄)⁺ | | |
| M12 | 25.5 | 24.25 | 636 (M + NH₄)⁻ | 618 | HLM, MKLM, MLM, |
| | | | 542 (¹³C − M + NH₄)⁻ | | RLM |
| M13 | 27.5 | 25.25-26.5 | 620 (M + NH₄)⁻ | 602 | HLM, MKLM, MLM |
| | | | 626 (¹³C − M + NH₄)⁻ | | RLM, DLM |
| M13A | −29.5 | 28.25 | 576 (M + NH₄)⁻ | 558 | MKLM |
| | | | 582 (¹³C − M + NH₄)⁻ | | |
| M13B | −28.7 | 28.25 | 578 (M + NH₄)⁻ | 560 | MKLM |
| | | | 584 (¹³C − M + NH₄)⁺ | | |
| M13C | −35.6 | 34.5 | 620 (M + NH₄)⁻ | 602 | HLM, MKLM, MLM |
| | | | 626 (¹³C − M + NH₄)⁻ | | RLM, DLM |
| M13D | −36.1 | 34.75 | 618 (M + NH₄)⁺ | 600 | MKLM |
| | | | 624 (¹³C − M + NH₄)⁺ | | |
| M15A | −39.1 | 37.75 | 891 (M + NH₄)⁻ | 873 | MKLM |
| | | | 897 (¹³C − M + NH₄)⁺ | | |
| M15B | −39.6 | 38.25 | 909 (M + NH₄)⁺ | 891 | MKLM |
| | | | 915 (¹³C − M + NH₄)⁻ | | |
| M17 | −44.3 | 42.75 | 879 (M + NH₄)⁺ | 861 | MKLM |
| | | | 885 (¹³C − M + NH₄)⁻ | | |
| M18A | −48.5 | 46.5 | 835 (M + NH₄)⁻ | 817 | MKLM |
| | | | 841 (¹³C − M + NH₄)⁻ | | |
| M18B | −49.6 | 47.75 | 851 (M + NH₄)⁻ | 833 | MKLM |
| | | | 857 (¹³C − M + NH₄)⁺ | | |
| M18C | −51.5 | 49.75 | 833 (M + NH₄)⁺ | 815 | MKLM |
| | | | 839 (¹³C − M + NH₄)⁺ | | |
| M19 | −52.4 | 49.75 | 879 (M + NH₄)⁺ | 861 | MKLM |
| | | | 885 (¹³C − M + NH₄)⁻ | | |
| M19A | −53.3 | 51.5 | 851 (M + NH₄)⁻ | 833 | MKLM |
| | | | 857 (¹³C − M + NH₄)⁻ | | |
| M19B | −53.5 | 51.5 | 877 (M + NH₄)⁻ | 859 | MKLM |
| | | | 883 (¹³C − M + NH₄)⁺ | | |
| M20 | −56.8 | 55-55.5 | 877 (M + NH₄)⁻ | 859 | HLM, MKLM, MLM |
| | | | 883 (¹³C − M + NH₄)⁺ | | DLM |
| M21 | −58.4 | 56.75-57.0 | 875 (M + NH₄)⁻ | 857 | HLM, MKLM, MLM |
| | | | 881 (¹³C − M + NH₄)⁻ | | RLM, DLM |
| M22 | −58.4 | 56.75-57.0 | 893 (M + NH₄)⁻ | 875 | HLM, MKLM, MLM |
| | | | 899 (¹³C − M + NH₄)⁻ | | RLM, DLM |
| M23 | −61.1 | 59.5-60.0 | 879 (M + NH₄)⁻ | 861 | HLM, MKLM MLM |
| | | | 885 (¹³C − M + NH₄)⁻ | | RLM, DLM |
| M20B | −63.7 | 62.75 | 877 (M + NH₄)⁻ | 859 | HLM, MLM, DLM |
| | | | 883 (¹³C − M + NH₄)⁻ | | RLM |
| M21B | −63.8 | 62.75-63.0 | 875 (M + NH₄)⁻ | 857 | HLM, MKLM |
| | | | 881 (¹³C − M + NH₄)⁺ | | MLM, DLM |
| M24 | −63.8 | 62.75-63.0 | 893 (M + NH₄)⁺ | 875 | HLM, MKLM, MLM |
| | | | 899 (¹³C − M + NH₄)⁺ | | RLM, DLM |
| M23A | −64.5 | 63.8 | 879 (M + NH₄)⁺ | 861 | RLM, MLM, DLM |
| | | | 885 (¹³C − M + NH₄)⁻ | | |
| M25 | −64.6 | 64.0 | 907 (M + NH₄)⁻ | 889 | HLM, MKLM, DLM |
| | | | 913 (¹³C − M + NH₄)⁺ | | |
| M23B | −65.0 | 64.25-64.5 | 879 (M + NH₄)⁺ | 861 | RLM, MLM, DLM |
| | | | 885 (¹³C − M + NH₄)⁻ | | |
| M23C | −65.3 | 64.25-64.5 | 879 (M + NH₄)⁺ | 851 | MLM, DLM |
| | | | 885 (¹³C − M + NH₄)⁻ | | |
| M26 | −65.4 | 64.5-64.75 | 821 (M + NH₄)⁺ | 803 | HLM, MKLM, MLM |
| | | | 827 (¹³C − M + NH₄)⁺ | | RLM, DLM |
| M23D | −65.6 | 63.75 | 879 (M + NH₄)⁺ | 861 | MKLM, MLM, DLM |
| | | | 885 (¹³C − M + NH₄)⁺ | | |
| Parent | 65.4 | 65.25-65.5 | 869 (M + NH₄)⁺ | 845 | HLM, MKLM, MLM |
| | | | 869 (¹³C − M + NH₄)⁻ | | RLM, DLM |
| M27³ | 85.5 | 86.5 | 863 (M + NH₄)⁺ | 845 | HLM, MKLM, MLM |
| | | | 869 (¹³C − M + NH₄)⁺ | | RLM, DLM |

¹Retention time by TSQ Radioactive Detector.
²Retention time by Reconstructed HPLC Chromatogram; it was expressed as a region
³7-epi-Compound I

The abbreviations are used in the following example relating to the metabolites obtained in the biotransformation of [¹⁴C] Compound I in bile duct cannulated rats.

### List of Abbreviations

| | |
|---|---|
| CH₃CN | Acetonitrile |
| BDC | Bile duct cannulated |
| CH₃COONH₄ | Ammonium acetate |
| DPM | Disintegrations per minute |
| HCOOH | Formic acid |
| HPLC | High-performance liquid chromatography |
| JVC | Jugular vein cannulated |
| LC/MS/MS | Liquid chromatography-tandem mass spectrometry |
| LC/RAM/(+)ESI | Liquid chromatography/radioactivity monitoring/ |
| | positive ion electrospray ionization |
| LC/RAM/(−)ESI | Liquid chromatography/radioactivity monitoring/ |
| | negative ion electrospray ionization |
| LC/RAM/ | Liquid chromatography/radioactivity monitoring/ |
| APICID/(+)ESI | atmospheric pressure in-source collision induced |
| | dissociation/positive ion electrospray ionization |
| LSC | Liquid scintillation counting |
| M | Metabolite |
| CH₃OH | Methanol |
| Rₜ | Retention time |
| ROI | Region of Interest |

A single 25-mg/kg oral dose of [¹⁴C] Compound I, as a solution in cremophore

EL/ethanol/water (10:10:80, v/v/v), was separately administered to two male bile duct cannulated (BDC) rats. Within 24 hr after dosing, an average of 25.8% of the administered dose was excreted in bile, The corresponding value in urine was 3.9%. Total recovery of the administered dose in bile and urine averaged 29.6%. Thus, the oral absorption of [¹⁴C] Compound I in rats was estimated to be at least 30%. Following the oral dose, the concentration of total radioactivity in plasma was measurable up to 24 hr and averaged 349 ng-equiv./mL at that time. The parent drug was a prominent radioactive component in plasma. At 24 hr after dosing, the concentration of Compound I in plasma was estimated to be 61 ng/mL. More than 20 hydrolytic and oxidative metabolites of [¹⁴C] Compound I were detected in plasma.

The profiles of radioactive components in pooled bile and urine samples were determined by HPLC radiochromatography. The major radioactive metabolites in selected bile and urine samples were analyzed by LC/RAM/(+)ESI- and LC/RAM(−)ESI-MS and MS/MS. The results showed that the absorbed [¹⁴C] Compound I was extensively metabolized. Little parent drug (<1% of the dose) was excreted in bile and urine. Twenty-three metabolites in bile and four metabolites in urine were identified or characterized. Together they accounted for 77.6% and 80% of total radioactivity (19.9% and 3.1% of the dose) in the 0-24 hr bile and urine samples, respectively. No single major metabolite was observed in bile. [¹⁴C]Hippuric acid (M3), which was solely excreted in urine, alone accounted for ˜56% of total radioactivity in urine or ˜2.3% of the dose. The identified metabolites could be classified into two categories: one derived from cleavage of the side chain at the C₁₃-position of [¹⁴C] Compound I followed by oxidation and glutathione conjugation of the formed baccatin moiety (Category I metabolites), and the other derived from direct oxidation and glutathione conjugation of [¹⁴C] Compound I (Category II metabolites). The metabolites of both categories were excreted in bile to about the same extent. Glutathione conjugation occurred primarily on the benzoyl group following the oxidation of the phenyl ring. Two metabolites derived from successive oxidations of the t-butyl ester group on the C₁₃-side chain of [¹⁴C] Coompound I followed by spontaneous cyclization of the putative aldehyde and acid derivatives were identified.

### Introduction

The objective of this study was to investigate the metabolic profile of the test article in bile, urine, and plasma and to identify the prominent metabolites by LC/MS/MS following a sinle 25-mg/kg oral dose of [¹⁴C] Compound I to BDC/JVC rats.

### Materials

Test Article
1. Radiolabeled and Non-radiolabeled Test Articles

| | |
|---|---|
| Compound Name: | Compound I |
| Empirical Formula: | C₄₃H₅₉NO₁₆ |
| Molecular Weight: | 845.95 |
| Compound ID's: | [¹⁴C]-labeled: [¹⁴C] Compound I-03 |
| | Non-radiolabeled: Compound I-01 |
| | [¹³C]-labeled: [¹³C] Compound I-02 |
| Lot Numbers: | [¹⁴C] Compound I: 001 |
| | Compound I: C038B |
| | [¹³C] Compound I: 001 |
| Radiochemical Purity: | 95.9% (Determined by HPLC in XBL) |
| Specific Activity: | 26.1 μCi/mg |

Radiolabeled, non-radiolabeled, and [¹³C]-labeled test articles used in the study were provided by the sponsor.

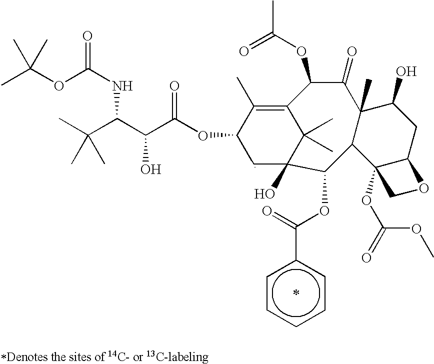

The radiochemical purity of the test article in the dosing solution was confirmed by HPLC prior to use.

Non-radiolabeled Compound I 01 (purity: 99.8%) and [¹³C]Compound I(purity: >99%) were used in this study as a reference chemical and/or to isotopically dilute the [¹⁴C] Compound I.

2. Storage and Disposition

Radiolabeled ([¹⁴C]), non-radiolabeled, and [¹³C]-labeled test articles were stored in a −20° C. freezer.

### Animals

Bile duct and jugular vein cannulated (BDC/JVC) male Sprague-Dawley rats (Hla®: [SD] CVF®, 6-10 weeks old and weighing 300±50 g at dosing), were purchased from Hilltop Laboratories (Scottdale, Pa.). Upon receipt, each rat was marked on the tail and given an ear tag with a unique identification number.

1. Acclimation

All animals were acclimated for at least 4 days in individual polycarbonate cages before dosing. During acclimation, the animals were examined every day for any signs of abnormalities indicative of health problems. One day prior to dosing, animals were housed in Nalgene metabolic cages to collect pre-dose urine samples and to acclimate the animals to the metabolic cages.

2. Housing and Maintenance

Environmental controls in the animal room were set to maintain a temperature of 19-25° C., a relative humidity of 50±20%, and intermittent light and dark cycles of ˜12 hours. Environmental condition records were maintained in the study file. Any variations from these conditions were documented.

Fresh tap water was available ad libitum through a bottle dispenser attached to each cage. During bile collection the rats were given an electrolyte-supplemented water ad libitum. Samples of the tap water were analyzed by International Hydronics Corporation, Rocky Hill, N.J., for selected organophosphates and chlorinated hydrcarbons.

Food was available to the rats ad libitrum throughout the acclimation period. The animals were fasted overnight prior to dosing and 6 hr post-dose. While the rats were housed in polycarbonate cages, they received Certified Rodent Chow® #5002 (PMI Feeds, Inc) in pellet form. At 6 hr post-dose, the rats received Certified Rodent Chow® #5002 in powder form.

There were no known contaminants in the food or water that would have adversely affected the results of this study. See the study file for feed/water analysis reports.

### Chemicals and Materials

Cremophore EL (Sigma, Lot No. 128H0166) was provided by the sponsor. Acetonitrile, ethanol, and formic acid were obtained from EM Science. Ammonium acetate was purchased from J. T. Baker. Water was produced on site through a NANOPure® II (Barnstead Co.) water purification system. Ready Value™ liquid scintillation cocktail was purchased from Beckman Instruments, Inc.

### Equipment and Instruments

Beckman Model TJ-6 Centrifuge

HPLC System, Waters Division of Millipore, with a Ranytest Ramona-5 Flow-through Radioactivity Monitor

HPLC System, Waters Division of Millipore, with a Packard flow Scintillation analyzer

Liquid Scintillation Counters; Models LS 3801, LS 5000TD, LS600TA; Beckman Instruments, Inc.

Finnigan MAT TSQ-7000 High Performance Quadrupole Mass Spectrometer

Finnigan MAT LCQ Ion Trap Mass Spectrometer

N-Evap® High-speed Analytical Evaporator (12 pasition), Model 111, Organomation Associates. Inc,

Ohaus® Electronic Balances, Models TP200S, GT410, EORW60; Ohaus Scale Corporation

Sartorius Balances, A120S. BP2100, BP310S, and BP210D

THDx Temperature and Humidity Recorder

### Methods

Determination of the Radiochemical Purity of [¹⁴C] Compound I

The radiochemical purity and the stability of [¹⁴C] Compound I in the dosing solution before and after dosing were determined by HPLC radiochromatography using the following system:

| | |
|---|---|
| HPLC System: | Waters Model 2690 Separation Module |
| UV Detector: | 996 PDA at 230-nm wavelength, 1.0 AUFS |
| Radioactivity Detector: | Raytest Ramona-5 Flow-through Radioactivity |
| | Monitor with a 200-μL glass cell |
| Integrator/Data System: | Millennium 32 data system |
| Column: | Ace 5 C18, 4.6 × 250 mm, 5 μm |
| | (Series #A2518) |
| Column Temperature: | 30° C. |
| Guard Column: | RP-8, 5 μm, 4.6 × 30 mm |
| Mobile Phase: | A: 0.1% HCOOH |
| | B: CH₃OH |
| Gradient System: | 50% A, 15 min to 73% B (15 min), 5 min to |
| | 100% B (5 min), 2 min to initial |
| | (Total run time = 42 min) |
| Flow Rate: | 1 mL/min |
| Autosampler | Ambient |
| Temperature: | |
| Fraction Collector: | Foxy 200 Fraction Collector |

Fractions of the effluent were collected (0.5 min/fraction) and the radioactivity in each fraction was determined by liquid scintillation counting.

Preparation of Dosing Solution

The dosing solution was prepared as follows: 7.96 mg of non-radiolabeled Compound I, 10.16 mg of [¹³C] Compound I and 2.99 mg of [¹⁴C] Compound I (77.7 μCi of radioactivity) were weighed into a vial. The compounds were then dissolved in 1.6 mL of a cremophore EL/ethanol solution (1:1, v/v) and diluted with 6.4 mL of sterile water. The total weight of the dosing solution was 7.53 g. The concentration of [¹⁴C] Compound I in the dosing solution was 2.80 mg/g (10.32 μCi/g) of solution. The nominal specific activity was 3.68 μCi/mg (8170 dpm/μg).

Dose Administration and Sample Collection

Two rats, housed individually in Nalgene metabolic cages, were each given a single 25-mg/kg nominal dose of [¹⁴C] Compound I orally by gavage. The actual amount of dosing solution administered to each animal was determined by weighing the loaded dosing syringe before dose administration and the emptied syringe after dose administration. Bile was collected from each animal between −1-0 (pre-dose), 0-6, 6-12, and 12-24 hr after dosing. Urine was collected between −24-0 (predose), 0-12, and 12-24 hr after dosing. Both the bile and urine samples were collected in tubes set on dry ice. At 0.5, 6, and 12 hr after dosing, blood samples (−1.2 mL) were collected from the jugular vein cannula into a heparinized syringe. At 24 hr after dosing, animals were sacrificed by CO₂ asphyxiation. Terminal blood was collected by cardiac puncture into a heparinized vacutainer. Predose blood sample was collectd from a separate control animal. Blood was centrifuged at 2,500 rpm and 4° C. for 10 min to separate the plasma. Bile, urine, and plasma samples were weighed and stored at −20° C. prior to analysis.

Determination of Radioactivity

Concentrations of radioactivity in bile, urine, and plasma samples were determined by counting duplicate aliquots of each sample in 5 mL of Ready Value™ cocktail (Beckman Instrunents, Inc.) with a Beckman LS 3801, LS 5000TD, or LS6000TA Liquid Scintillation Counter for 10 min or until the 2-sigma error was less than or equal to 2%, whichever came first. Quench correction was performed using an external standard method. Most of the HPLC fractions used to prepare reconstructed HPLC chromatograms were counted for 5 minutes except for plasma samples, which were counted for 10 minutes.

Preparation of Pooled Bile and Urine Samples for HPLC Analysis

A portion (−20% of each sample) of the 0-6, 6-12, and 12-24 hr bile samples from two rats were pooled by interval to prepare three pooled samples. A portion (−20% of each sample) of the 0-6, 6-12, and 12-24 hr bile samples from two rats were pooled together to prepare a 0-24 hr sample. Duplicate 10-μL aliquots of each pooled sample were taken for radioactivity counting.

A portion (−20% of each sample) of the 0-12 and 12-24 hr urine samples from two rats were pooled by interval to prepare two pooled samples. A portion (−20% of each sample) of the 0-12 and 12-24 hr urine samples from two rats were pooled together to prepare a 0-24 hr sample. Duplicate 10-μL aliquots of each pooled sample were taken for radioactivity counting.

Each pooled sample was directly analyzed by HPLC radiochromatography.

Preparation of Plasma Samples for HPLC Analysis

The 1, 6, 12, and 24 hr plasma samples from two rats were pooled correspondingly. A single aliquot (20 μL from the 1, 6, and 12 hr pooled samples and 40 μL from the 24 hr pooled sample) was taken for radioactivity counting. The remaining samples were extracted twice with an equal volume of CH₃CN each time. The CH₃CN extracts were pooled and a single 50-μL aliquot was taken from the pooled extract for counting. The pooled extracts were then evaporated to dryness under nitrogen at −40° C. Residues were dissolved in a small volume (250 μL) of methanol/water (1:1, v/v) mixture. A single 20-μL or 40-μL aliquot of the reconstituted solutions was taken for LSC. A portion (100 μL) of the reconstituted solution was used for the determination of metabolite profiles by HPLC radiochromatograpby.

Determination of Metabolite Profiles

The metabolite proflies of [¹⁴C]Compound I in bile, urine, and the CH₃CN extract of plasma were determined by HPLC radiochromatography using the following system:

| | |
|---|---|
| HPLC System: | Waters Model 2690 Separation Module |
| UV Detector: | 996 PDA at 254-nm wavelength, 1.0 AUFS |
| Radioactivity | Raytest Ramona-5 Flow-through Radioactivity |
| Detector: | Monitor with a 200-μL glass cell |
| Integrator/Data | Millennium 32 data system |
| System: | |
| Column: | Ace 5 C18, 4.6 × 250 mm, 5 μm (Series #A3538) |
| Column | 30° C. |
| Temperature: | |
| Guard Column: | RP-8, 5 μm, 4.6 × 30 mm |
| Mobile Phase: | A: 0.01 M CH₃COONH₄ (adjust pH to 3 with |
| | HCOOH), |
| | B: CH₃OH |
| Gradient | 100% A, 3 min to 40% B, 30 min to 60% B (15 min), |
| System 1: | 10 min to 65% B, 5 min to 100% B (9 min), 2 min to |
| | initial (Total run time = 74 min) |
| Gradient | 100% A, 3 min to 40% B, 30 min to 60% B (15 min), |
| System 2: | 10 min to 65% B, 5 min to 100% B (15 min), 2 min |
| | to initial (Total run time = 80 min) |
| Flow Rate: | 1 mL/min |
| Autosampler | 4° C. |
| Temperature: | |
| Fraction | Foxy 200 Fraction Collector |
| Collector: | |
| Integrator/Data | Millennium³² data system |
| System: | |

Gradient System 1 was used for analysis of the 0-6, 6-12, and 12-24 hr pooled bile samples and for the 0-12 and 12-24 hr pooled urine samples. Gradient System 2 was used for analysis of the 0-24 hr pooled bile and urine as well as for all of the plasma samples.

The radiochemical purity and the stability of [¹⁴C] Compound I in the dosing solution before and after dosing were initially determined using a different gradient condition and were subsequently re-determined using Gradient System 1 as described above.

Fractions of the effluent (0.5 min/fraction) were collected and the radioactivity in each fraction was determined by liquid scintillation counting.

Identification of Metabolites by LC/MS and LC/MS/MS

Metabolites of [¹⁴C] Compound I in selected bile and urine sanples were analyzed by LC/RAM/(+)ESI-MS and MS/MS, LC/RAM/(+)APICID/(+)EST-MS, and LC/RAM/(−)ESI-MS and MS/MS. The following HPLC system and conditions were used:

LC/MS Condition I

| | |
|---|---|
| HPLC System: | Waters 2690 Separations Module |
| UV Detector: | Waters 486 Tunable Absorbance Detector |
| Radioactivity | Raytest Ramona-5 Flow-through Radioactivity |
| Detector: | Monitor with a 110-μL glass cell |
| Column: | Ace 5 C18, 4.6 × 250 mm, 5 μm (Series #A3538) |
| Column Temperature: | 30° C. |
| Guard Column: | RP-8, 5 μm, 4.6 × 30 mm |
| Mobile Phase: | A: 0.01 M CH₃COONH₄ (adjust pH to 3 with |
| | HCOOH), |
| | B: CH₃OH |
| Gradient System 1: | 100% A, 3 min to 40% B, 30 min to 60% |
| | B (15 min), 10 min to 65% B, 5 min to 100% |
| | B (9 min), 2 min to initial |
| | (Total run rime = 74 min) |
| Gradient System 3: | 100% A, 60 min to 60% B, 5 min to 65% B, |
| | 5 min to 100% B (5 min), 2 min to initial |
| | (Total run time = 77 min) |
| Flow Rate: | 1 mL/min |
| Autosampler | 4° C. |
| Temperature: | |

The column effluent went through the radioactive detector and then through the mass spectrometer. On some occasions, the column effluent by-passed the radioactive detector and went directly to the mass spectrometer.

The HPLC system was interfaced to a Finnigan MAT TSQ-7000 High-Performance Quadrupole Mass Spectrometer using the following conditions:

| | |
|---|---|
| Mass Spectrometer: | Finnigan MAT TSQ-7000 High Performance |
| | Quadrupole Mass Spectrometer |
| Ionization Mode: | Positive or Negative Electrospray |
| Ion Spray (IS): | 4.5 kV |
| Capillary Temperature: | 240° C. |
| Sheath Gas Flow: | 80 psi |
| Auxiliary Gas Flow: | 20 mL/min |
| Collision Gas (CAD): | Argon |

LC/MS Condition II

| | |
|---|---|
| HPLC System: | Waters 2690 Separations Module |
| UV Detector: | Waters 486 Tunable Absorbance Detector |
| Radioactivity | Packard Flow Scintillation Analyzer |
| Detector: | with a 177-μL glass cell |
| Column: | Ace 5 C18, 4.6 × 250 mm, 5 μm (Series #A3454) |
| Column | 30° C. |
| Temperature: | |
| Guard Column: | RP-8, 5 μm, 4.6 × 30 mm |
| Mobile Phase: | A: 0.01 M CH₃COONH₄ (adjust pH to 3 with |
| | HCOOH), |
| | B: CH₃OH |
| Gradient | 100% A, 3 min to 40% B, 30 min to 60% B (15 min), |
| System 1: | 10 min to 65% B, 5 min to 100% B (9 min), 2 min to |
| | initial. |
| Flow Rate: | 1 mL/min |
| Autosampler | 4° C. |
| Temperature: | |

The column effluent was split between the flow scintillation analyzer and mass spectrometer.

The HPLC system was interfaced to a Finnigan LCQ Ion Trap Mass Spectometer using the following conditions:

| | |
|---|---|
| Mass Spectrometer: | Finnigan LCQ Ion Trap |
| | Mass Spectrometer |
| Ionization Mode: | Positive |
| Ion Spray (IS): | 5 kV |
| Capillary Temperature: | 200° C. |
| Sheath Gas Flow: | 80 psi |
| Auxiliary Gas Flow: | 20 mL/min |
| Collision Gas (CAD): | Nitrogen (N₂) |
| Relative Collision Energy: | 22% |
| Isolation Width for MS/MS: | 10 amu |

LC/MS Condition III

The same conditions as for Condition II were used except that an APICID source was activated.

Source Energy: 10 V

Metabolites were analyzed by all three LC/MS conditions (Conditions I, II, and III). The best results from one of the three conditions are presented.

Metabolite M13

One of the major metabolites in bile with a R₁ of −27 min was designated as M13. M13 exhibited (M+NH₄)⁺ and (¹³C−M+NH₄)⁺ ions at m/z 620 and m/z 626, respectively, indicating a molecular weight of 602 daltons which was 243 amu lower than that of Compound I (MW 845). The characteristic ions at m/z 327, 345, 525, and 105 suggested that the compound has a similar structure to that of the baccarin ring. Based on the data, M13 was proposed as a hydrolytic product of Compound I resulting from a cleavage of the side chain. M13 was also found in the 0-12 hr pooled urine.

Metabolites M4 and M5

Both M4 and M5 exhibited (M+NH₄)⁺ and (¹³C−M+NH₄)⁺ ions at m/z 926 and m/z 932, respectively, indicating a molecular weight of 908 daltons which was 306 amu higher than that of a cleavage product of Compound 1 (M13: MW 602). The two compounds were considered to be isomers. Both compounds were poorly ionized by TSQ but yielded an enhanced ionization by APICID(+)ESI using LCQ (LC/MS Condition III). Both compounds were subjected to tandem mass spectrometry (MS/MS) using both LCQ and TSQ. The characteristic ion at m/z 306 indicated that the compound contained a glutathione moiety. The ion at m/z 796 resulted from the loss of glutamic acid moiety from glutathione. The ion at m/z 428 resulted from S-glutathionyl-benzoic acid moiety. In addition to the ions at m/z 428 and 306, the spectrum also shows a characteristic ion at m/z 177, which resulted from the loss of glutamyl moiety from the glutathione conjugate. Thus, M4 and M5 were proposed as two isomers of the glutathione conjugate of M13. The proposed strucures of M4 and M5 are as follows: 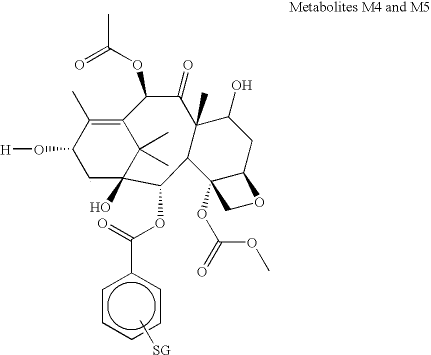
Metabolites M1 and M2

Both M1 and M2 exhibited (M+NH₄)⁺ and (¹³C−M+NH₄)⁺ ions at m/z 942 and m/z 948, respectively, indicating a molecular weight of 924 daltons which was 372 amu higher than that of M13 (MW 602). Both metabolites were 16 mass units higher than M4 and M5, indicating the addition of one oxygen to M4 and/or M5. Both compounds were also poorly ionized by TSQ as was observed with M4 and M5 but yielded improved ionization by APICID(+)ESI/LC using LCQ (LC/MS Condition III). Both compounds were then subjected to tandem mass spectomety (MS/MS) using both LCQ and TSQ. The characteristic ion at m/z 306 indicated that both compounds contained a glutathione moiety. The ion at m/z 812 resulted from the loss of a gluntic acid moiety. The ion at m/z 830 resulted from the loss of NH₃, H₂O, and a HCO₃CH₃ group. The MS/MS spectrum of M1 by LCQ also yielded a characteristic ion at m/z 428 corresponding to the S-glutathionyl benzoic acid moiety as was shown for M4 and M5, indicating that the phenyl group was not oxidized. The spectrum of M1 also showed characteristic ions at m/z 924, 906, and 888, each resulting from the loss of NH₃, followed by loss of one H₂O molecule or two H₂O molecules. The TSQ-MS/MS of M1 yielded additional ions at m/z 177 and m/z 385. The ion at m/z 385 was considered to result from the further loss of glutathionyl-benzoyl moiety and one H₂O molecule from the ion at m/z 830. Based on the information generated by the MS/MS data, the structure of M1 was proposed as follows:

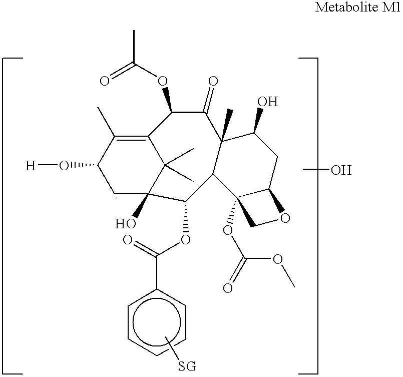

The LCQ MS/MS spectrum of M2 yielded a characteristic ion at m/z 444 resulting from the presence of S-glutathionyl-hydroxy-benzoic acid moiety as was illustrated for M4 and M5, indicating that an oxygen was added to the phenyl group. In addition to the ions at m/z 444 and m/z 306, the spectrum also shows a characteristic ion at m/z 177 which resulted from the loss of glutamyl moiety of glutathione. Thus, M2 was proposed as a glutathione conjugate of an oxidative product of M13 and the structure is shown as follows: 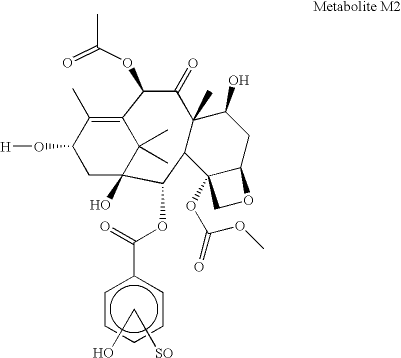
Metabotite M3

Metabolite M3 was a major metabolite in urine but was not detected in bile. This compound was not ionized under positive ionization mode, therefore, negative ionization mode was used to analyze the compound. In order to better separate the compound from the co-eluting matrices to assist the ionization of the compound, Gradient System 3 was used for analysis M3 exhibited (M−H)⁻ and (¹³C−M−H)⁻ ions at m/z 178 and m/z 184, respectively, indicating a molecular weight of 179 daltons. After identification of molecular ions of the compound, the urine sample was analyzed under HPLC Gradient System I to show how Gradient System 3 increased the ionization of the compound. The ion at m/z 184 was far less intense than the ion at m/z 178. Both of the m/z 178 and m/z 184 ions were subjected to LC/(−)ESI-MS/MS analysis. The (¹³C−M−H)⁻ ion at m/z 184 produced two prominent fragment ions at m/z 139 and m/z 82. These ions were 6 amu higher than the corresponding fragment ions at m/z 133 and m/z 76 derived from the (M−H)⁻ ion at m/z 178. These results confirm that the compound is a metabolite derived from Compound I. Based on the fragmentation pattern and the molecular weight, the compound was proposed to be hippuric acid. The isotope ratio should have been approximately 1:1 assusing the compound was derived solely from Compound I. Since hippuric acid could also be derived from any other compound containing a benzoyl moiety, it was considered that the majority of the ion at m/z 178 could have been derived from other xenobiotic compounds containing a benzoyl moiety, such as those present in the rat feed. The proposed structure of M3 and its MS fragmentation is shown below: 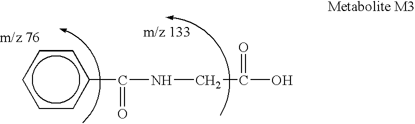
Metabolite M6

M6 exhibited (M+NH₄)⁺ and (¹³C−M+NH₄)⁺ ions at m/z 682 and m/z 688, indicating a molecular weight of 664 daltons which was 62 amu higher than that of M13 (MW 602) and suggesting the addition of four oxygens in conjunction with dehydrogenation and/or ring closure, or the addition of three oxygens and a methyl group. The ion at m/z 647 resulted from the loss of NH₃ and H₂O from the ion at m/z 682. The characteristic ion at m/z 587 resulted from the loss of NH₃, H₂O, and CH₃COOH from the ion at m/z 682 and it is 62 amu higher than the corresponding fragment ion at m/z 525 of M13. The loss of another water molecule resulted in the ion at m/z 569. A further loss of HCO₃CH₃ from the most abundant fragment ion at m/z 587 could have resulted in the ion at m/z 511 which is 62 amu higher than the corresponding ion at m/z 449 of M13. The ion at m/z 343 resulted from an additional loss of 168 amu, which is the benzoic acid containing a hydroxy and a methoxy group, from the fragment ion at m/z 511. The ion at m/z 325 resulted from an additional loss of H₂O from the ion at m/z 343. The most distinctive ions at m/z 151 (from the ion at m/z 682) and m/z 157 (from the ion at m/z 688) were 46 amu higher than the corresponding ion at m/z 105 of M13, indicating the presence of a hydroxy and a methoxy group on the aromatic ring. The above data also indicated that one oxygen was added to the baccatin ring while 46 mass units in the form of a hydroxy and a methoxy group were added to the phenyl group. Based on the known metabolic pathways for aromatic hydrocarbons, the structure of M6 is proposed as follows:

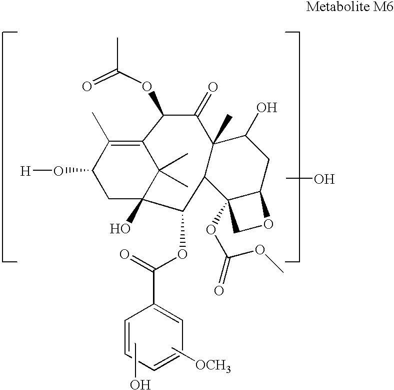

Although they are most probably in the ortho-position to each other, the positions of the hydroxyl group and the O-methyl group in the phenyl ring were not confirmed. The phenyl group was considered to form catechol metabolites by the epoxide-dihydrodiol pathway, which then undergo O-methylation.

Metabolite M7

Like M6, M7 also exhibited (M+NH₄)⁺ and (¹³C−M+NH₄)⁺ ions at m/z 682 and m/z 688, indicating a molecuar weight of 664 daltons which was 62 amu higher than that of M13 (MW 602). Similar fragment ions as of M6 were also found. However, the R₁ of M7 was different from that of M6. These data indicated that M7 may be an isomer of M6 with the hydroxyl and methoxy groups being added to different positions on the phenyl ring or 7-epi isomer of M6. The structure of M7 was proposed as follows: 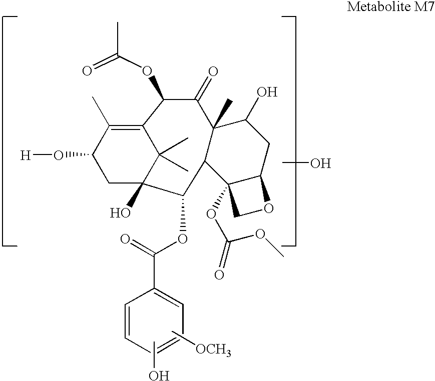
Metabolites M8 and M8A

M8 exhibited (M+NH₄)⁺ and (¹³C−M+NH₄)⁺ ions at m/z 652 and m/z 658, indicating a molecular weight of 634 daltons which was 32 amu higher than that of M13 (MW 602), suggesting the addition of two oxygens. The characterstic ion at m/z 599 resulted from the loss of a NH₃ and two H₂O from the ion at m/z 652. The ion at m/z 557 resulted from the loss of a NH₃, one H₂O, and CH₃COOH from the ion at m/z 652. The presence of a fragment ion at m/z 121 indicated an addition of one oxygen to the phenyl ring. The ions at m/z 325 and m/z 343 that were also found as the prominent fragment ions of M6 and M7 indicate that both ions resulted from loss of the benzoyl moiety. Based on these data, the structute of M8 was proposed as follows:

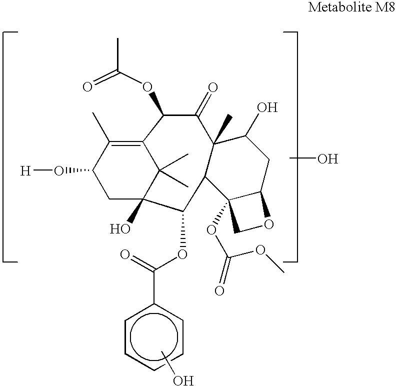

Another compound (M8A) at R₁=17.5 min also exhibited the (M+NH₄)⁺ and (¹³C−M+NH₄)⁺ ions at m/z 652 and m/z 658, respectively. It was detected at the shoulder of the M8 peak. M8A yielded the most prominent ion at m/z 599 resuting from the loss of NH₃ and two H₂O from the ion at m/z 652. The presence of a fragment ion at m/z 105 indicated that the phenyl ring was intact. Based on these data, the structure of M8A was proposed as follows: 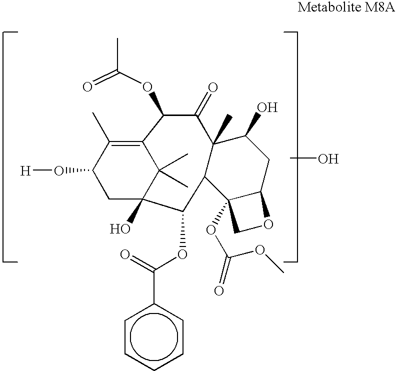
Metabolite M9

M9 exhibited (M+NH₄)⁺ and (¹³C−M+NH₄)⁺ ions at m/z 622 and m/z 628, indicating a molecular weight of 604 daltons which was 2 amu higher than that of M13 (MW 602) and suggesting the addition of two hydrogen atoms. The characteristic ion at m/z 527, which was 2 amu higher than the equivalent ion of M13, was derived from the loss of a NH₃, an H₂O, and a CH₃COOH from the ion at m/z 622. The ion at m/z 369 resulted from the loss of a NH₃, three H₂O, a CH₃COOH, and a C₆H₅COOH from the ion at m/z 622. The characteristic ion at m/z 389 was considered to have resulted from the loss of NH₃, H₂O, HCO₃CH₃, and C₆H₅COOH fragments from the ion at m/z 622. Based on this information and the enhanced polarity of the metabolite as compared to M13, the structure of M9 was proposed as follows, with the keto group at the C₉ position being reduced to a hydroxy group. 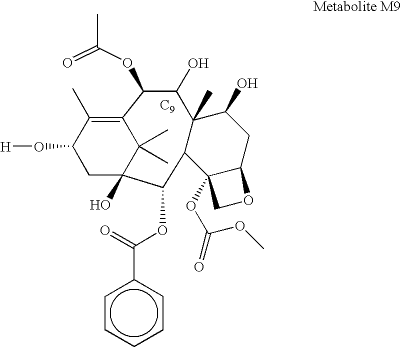
Metabolite M10

M10 exhibited (M+NH₄)⁺ and (¹³C−M+NH₄)⁺ ions at m/z 636 and m/z 642, indicating a molecular weight of 618 daltons which was 16 amu higher than that of M13 (MW 602) and suggesting the addition of one oxygen. In comparison to the fragmentation pattern of M13, the fragment ions at m/z 343, 361, 483, and 541 were 16 amu higher than the corresponding fragment ions at m/z 327, 345, 525, and 467, respectively, of M13. The ion at m/z 105 indicated that the phenyl ring was intact. Based on this information, the structure of M10 was proposed as follows: 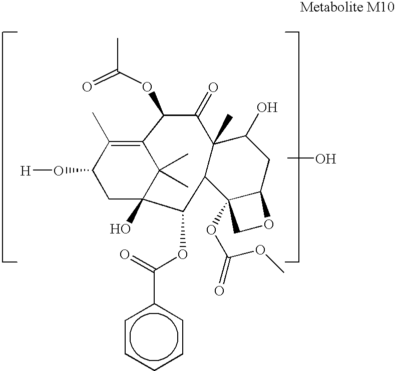
Metabolite M11

M11 exhibited (M+NH₄)⁺ and (¹³C−M+NH₄)⁺ ions at m/z 650 and m/z 656, indicating a molecular weight of 632 daltons which was 30 amu higher than that of M13 (MW 602) and suggesting the addition of two oxygens and the loss of two hydrogens. In comparison to the fragmentation pattern of M13, the prominent ions at m/z 555, 479, 435, and 357 were all 30 amu higher than the corresponding fragment ions a m/z 525, 449, 405, and 327, respectively, of M13. These data indicated that the benzoyl, acetyloxy, and methoxycarbonyloxy groups were all intact. Thus, the two oxygens were most likely added to the methyl group at the C₁₂ position. The characteristic ion at m/z 105 indicated that the phenyl group was also intact. Based on these data, the structure of M11 was proposed as follows: 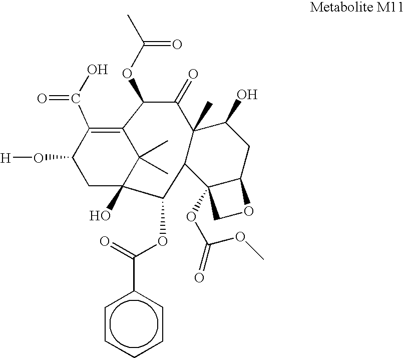
Metabolite M12

M12 exhibited (M+NH₄)⁺ and (¹³C−M+NH₄)⁺ ions at m/z 636 and m/z 642, indicating a molecular weight of 618 daltons which was 16 amu higher than that of M13 (MW 602) and suggesting the addition of one oxygen. In comparison to the fragmentation pattern of M13, the prominent ions at m/z 343, 361, and 541 were all 16 amu higher than the corresponding fragment ions of M13 suggesting that the benzoyl, acetyloxy, and methoxycarbonyloxy groups were all intact. The characteristic ion at m/z 105 further supported that the phenyl group was intact. The position of the added hydroxyl group was unknown but may have been at a position where a water molecule could easily be lost. M12 also had a longer retention time than M10, indicating that it is less polar than M10. A possibility is that M12 could be 7-epi isomer of M10. Based on these data, the structure of M12 was proposed as follows: 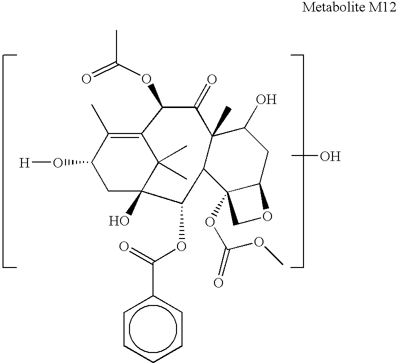
Metabolite M14

M14 exhibited (M+NH₄)⁺ and (¹³C−M+NH₄)⁺ ions at m/z 895 and m/z 901, indicating a molecular weight of 877 daltons which was 37 amu higher than that of Compound I (MW 845), suggesting the addition of two oxygens. The MS/MS spectra of M14 obtained by TSQ and LCQ both did not give any conclusive ions. No distinctive peaks resulting from the loss of a t-butyl group were observed, indicating the absence of free t-butyl group. On the other hand, there were no characteristic ions detected to show that the baccatin moiety was intact. M14 could have resulted from the addition of two oxygens in the form of two hydroxides or from oxidation of one of the methyl groups to form a carboxylic acid functionality together with reduction of C₉-ketone to form C₉H—OH. Based on this limited information, the structure of M14 was tentatively assigned as follows: 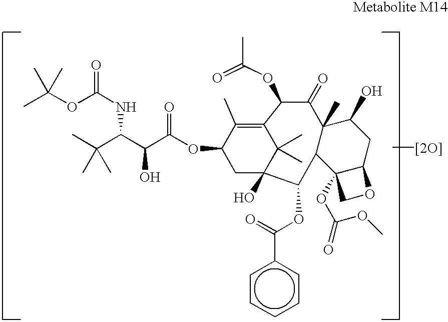
Metabolite M15

M15 exhibited (M+NH₄)⁺ and (¹³C−M+NH₄)⁺ ions at m/z 1169 and m/z 1175, respectively, indicating a molecular weight of 1151 daltons which was 306 amu higher than that of Compound I (MW 845). The compound was poorly ionized by TSQ. However, it yielded improved ionization by APICID(+)ESI using LCQ (LC/MS Condition III). The compound was subjected to tandem mass spectrometry (MS/MS) by using both LCQ (LC/MS Condition II) and TSQ (LC/MS Condition I). The characteristic ions at m/z 306, 177 and 130 indicated that the compound contained a glutathione moiety. The ions at m/z 846, 790, 746, 585, 525, and 387 were characteristic ions of Compound I, confirming that M15 was a glutathione conjugate of Compound I. The structure of M15 was proposed as follows: 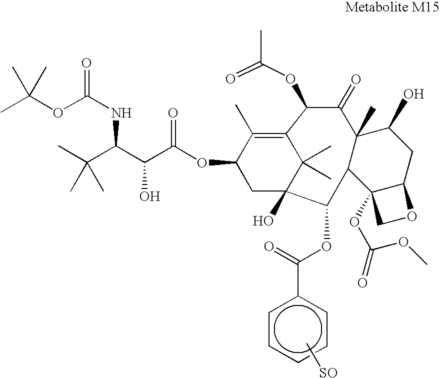
Metabolite M16

M16 exhibited (M+NH₄)⁺ and (¹³C−M+NH₄)⁺ ions at m/z 895 and m/z 901, indicating a molecular weight of 877 daltons which was 32 amu higher than that of Compound I (MW 845), suggesting the addition of two oxygens. The product was not well-ionized when analyzed by TSQ (LC/MS Condition I). The characterstic ion at m/z 821 resulted from the loss of a t-butyl group, indicating that at least one of the two t-butyl groups was intact. Based on the fact that the MS/MS spectrum of M16 did not yield any of the characteristic ions of Compound I, it was concluded that at least one of the two oxygens was added to the baccatin ring. M16 could have resulted from addition of two oxygens in the form of two hydroxides or from oxidation of one of the methyl groups to form a carboxylic acid functionality together with reduction of C₉-ketone to form C₉H—OH. Therefore, the structure of M16 was proposed as follows: 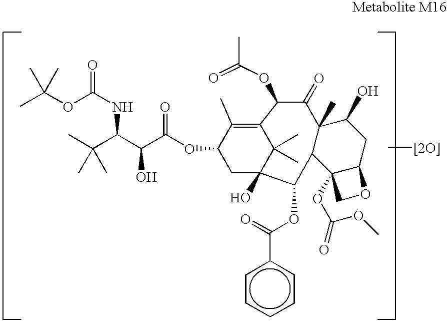
Metabolite M17

M17 exhibited (M+NH₄)⁺ and (¹³C−M+NH₄)⁺ ions at m/z 879 and m/z 885 respectively, indicating a molecular weight of 861 daltons which was 16 amu higher than the parent compound, suggesting the addition of one oxygen. The characteristic ions at m/z 585, 525, 403, 387, and 309 were also the characteristic ions of Compound I, suggesting that the baccatin moiety was intact. The characteristic ions at m/z 178 and 222 was 16 amu higher than the corresponding ions at m/z 162 and 206 of Compound I, suggesting that an oxygen was added to the t-butyl group on the side chain.

Although this data indicates that the baccatin moiety is not oxidized, it does not exclude the possibility that the 7-hydroxyl group is epimerized. Three metabolites (M17, M19, and M23) monohydroxylated on the side chain were identified by MS; there are only two unique positions (the two t-butyl groups) for monooxygenation on the side chain. This would indicate that one of them is an epimer of one of the other isomers. Epimerization of the 7-hydroxyl group has been observed for other taxanes and is most likely. The structure of M17 was proposed as follows: 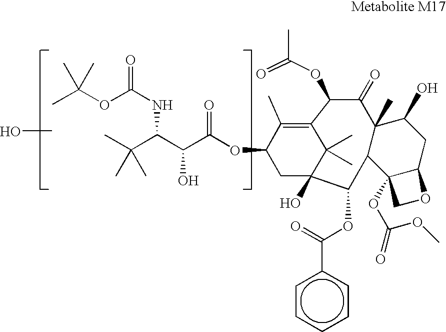
Metabolite M18

M18 exhibited (M+NH₄)⁺ and (¹³C−M+NH₄)⁺ ions at m/z 895 and m/z 901, indicating a molecular weight of 877 daltons which was 32 amu higher than that of Compound I (MW 845), suggesting the addition of two oxygens. The compound was not well ionized when analyzed by TSQ (LC/MS Condition I). The characteristic ions at m/z 601, 465, and 343 were 16 amu higher than the corresponding ions at m/z 585, 449, and 327, respectively, of Compound I, indicating that one of the two oxygens was added to the baccatin moiety. By deduction, the other oxygen must have been added to the side chain. The structure of M18 was thus proposed as follows: 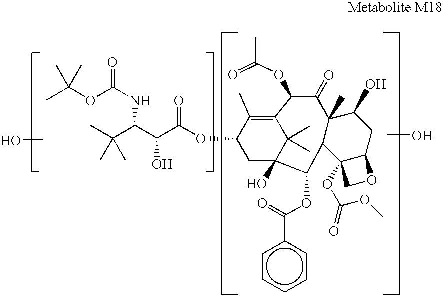
Metabolite M19

M19 exhibited (M+NH₄)⁺ and (¹³C−M+NH₄)⁺ ions at m/z 879 and m/z 885 respectively, indicating a molecular weight of 861 daltons which was 16 amu higher than that of Compound I, suggesting the addition of one oxygen. The characteristic ions at m/z 585, 525, 449, 387, 327, and 309 were also those of Compound I, suggesting that the baccatin moiety was intact. The characteristic ions at m/z 178 and m/z 222 were 16 amu higher than the corresponding ions at m/z 162 and m/z 206 of the parent drug, suggesting the addition of an oxygen to the side chain. This could be a 7-epi isomer of M17 or M23 (refer to the discussion for M17). The structure of M19 was proposed as follows: 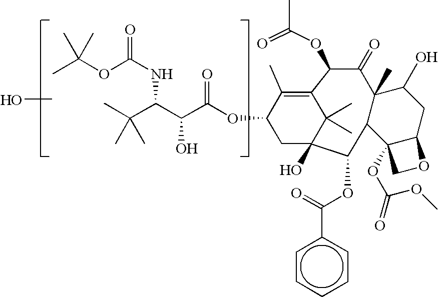
Metabolite M19
Metabolite M20

M20 exhibited (M+NH₄)⁺ and (¹³C−M+NH₄)⁺ ions at m/z 877 and m/z 883 respectively, indicating a molecular weight of 859 daltons which was 14 amu higher than that of the parent drug, suggesting the addition of one oxygen and the loss of two hydrogens. The characteristic ions at m/z 525, 467, and 309 were also those of Compound I, suggesting that the baccatin moiety was intact. M20 was also found in the 0-12 hr pooled urine sample. Based on the molecular weight, the characteristic ions which indicated that the baccatin moiety was intact as well as by analogy to docetaxel metabolism and with a metabolite (M20) which was subsequently identified as a ring-closure product of Compound I in human liver microsomes, the structure of M20 was proposed as follows:

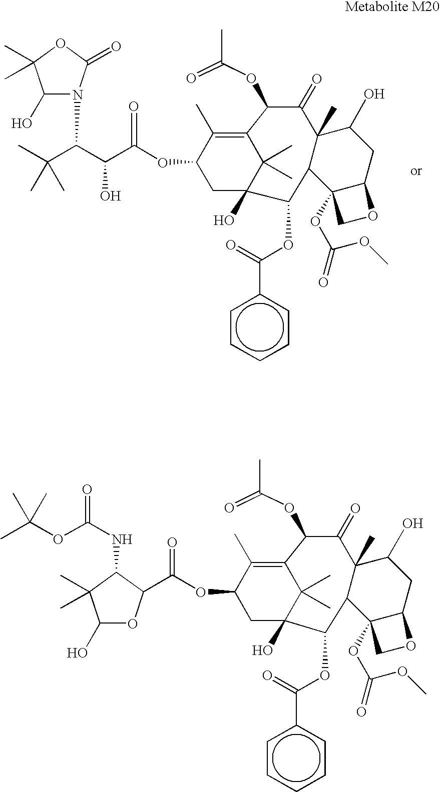

Since there was no distinctive fragment showing where cyclization occurred, the structure of M20 could be either of the two shown above.

Metabolite M21

M21 exhibited (M+NH₄)⁺ and (¹³C−M+NH₄)⁺ ions at m/z 875 and m/z 881 respectively, indicating a molecular weight of 857 daltons which was 12 amu higher than that of Compound I, suggesting the addition of one oxygen and the loss of 4 hydrogens. This metabolite was only detected in urine and was not further analyzed by MS/MS due to the limited amount of material. M21 was identified as one of the major metabolites of Compound I in human liver microsome incubation mixture by LC/MS/MS analysis and by analogy to docetaxel metabolism. Its structure was, therefore, proposed as follows: 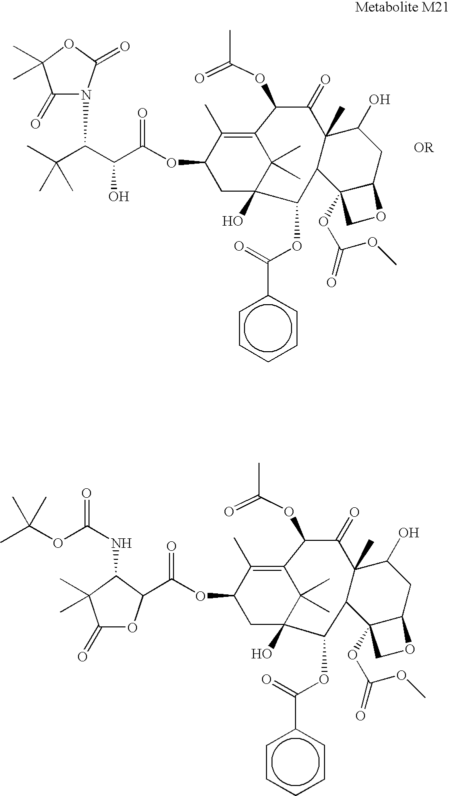
Metabolite M22

M22 exhibited (M+NH₄)⁺ and (¹³C−M+NH₄)⁺ ions at m/z 893 and m/z 899 respectively, indicating a molecular weight of 875 daltons which was 30 amu higher than that of the parent compound, suggesting the addition of two oxygens and the loss of two hydrogens. The characteristic ions at m/z 585, 525, 449, 405, 387, 327, and 309 were also those of Compound I, suggesting that the baccatin nolety was intact. The characteristic ions at m/z 192, 236, and 292 were 30 amu higher than the corresponding ions at m/z 162, 206, and 262 of the parent drug. The ions at m/z 236 and 192 resulting from the loss of C₄H₈ from the ion at m/z 292 and from further loss of CO₂ indicated that both oxygens were added to the t-butyl group (on 3′-carbon) of the side chain, followed by the removal of two hydrogens. Thus, the structure of M22 is proposed as follows: 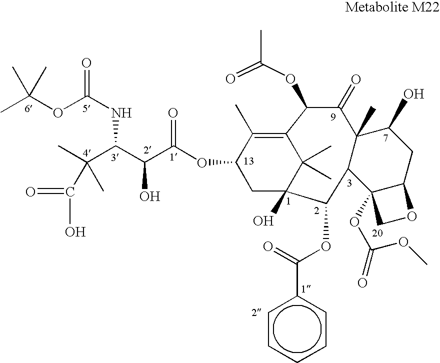
Metabolite M23

M23 exhibited (M+NH₄)⁺ and (¹³C−M+NH₄)⁺ ions at m/z 879 and m/z 885 respectively, indicating a molecular weight of 861 daltons which was 16 amu higher than that of the parent compound, suggesting the addition of one oxygen. The presence of the ions at m/z 585 and 525 indicated that the baccatin ring was intact. The characteristic ions at m/z 278 and 222 (16 amu higher than the ions at 262 and 206 of Compound I) indicated that the oxidation occurred on the side chain. This could be a 7-epi isomer of M17 or M19 (refer to the discussion for M17). The structure of M23 was proposed as follows: 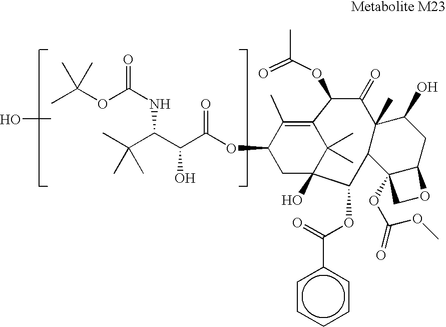
Metabolite M24

M24 exhibited (M+NH₄)⁺ and (¹³C−M+NH₄)⁺ ions at m/z 893 and m/z 899 respectively, indicating a molecular weight of 875 daltons which was 30 amu higher than that of the parent compound, suggesting the addition of two oxygens and the loss of two hydrogens. M24 and M22 had the same molecular weight and might be epimers. No MS/MS data could be obtained due to the limited amount of the compound. M24 was also found and identified as one of the major metabolites of Compound I formed by human liver microsomes, which had a similar R₁ and MS spectrum as those of the rat metabolite. The stucture of M24 was proposed as follows:

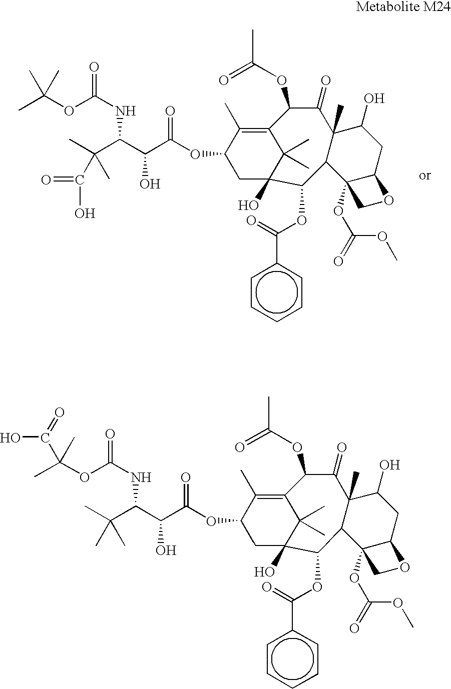

The possibility that M24 could be a 7-epi isomer of M22 also cannot be excluded.

Other minor metabolites in pooled bile and urine samples were not identified due to insufficient quantities of material in the samples.

Metabolite Profiles in Plasma

The pooled plasma samples from two rats were extracted with acetonitrile and the extraction was quantitative. The profiles of radioactive metabolites in the concentrated extracts were determined by HPLC radiochromatography.

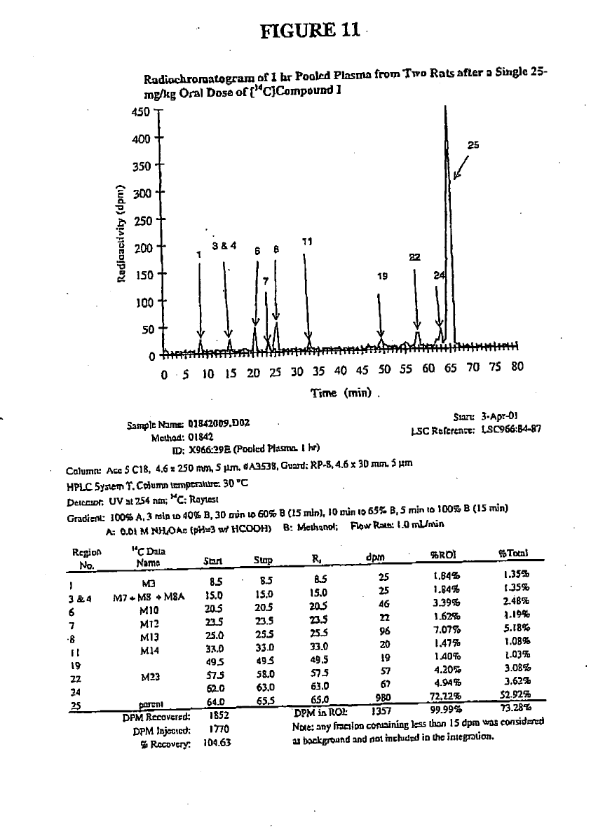

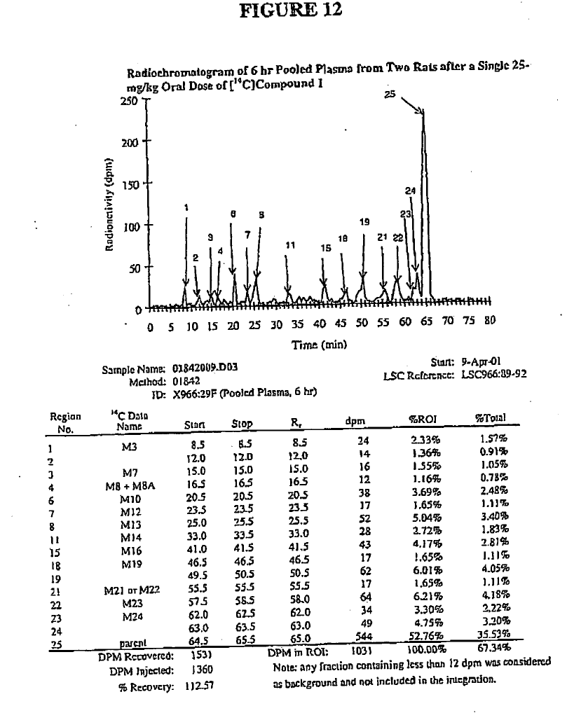

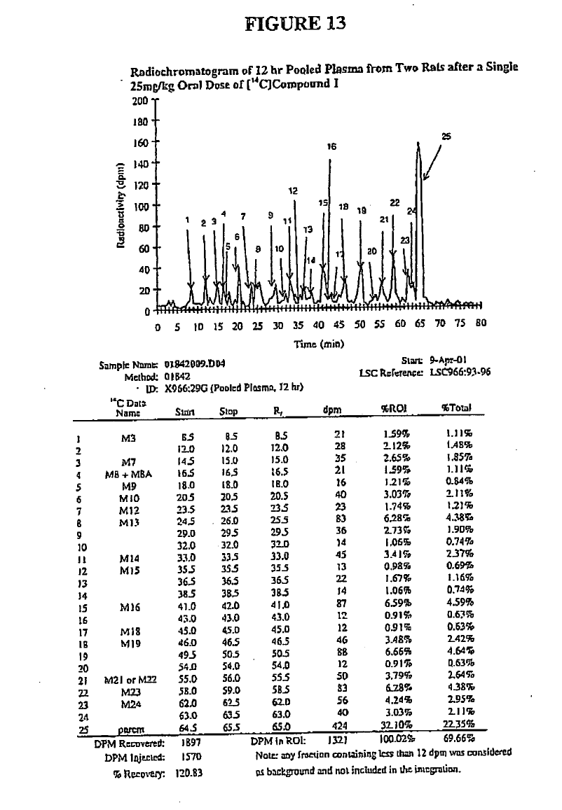

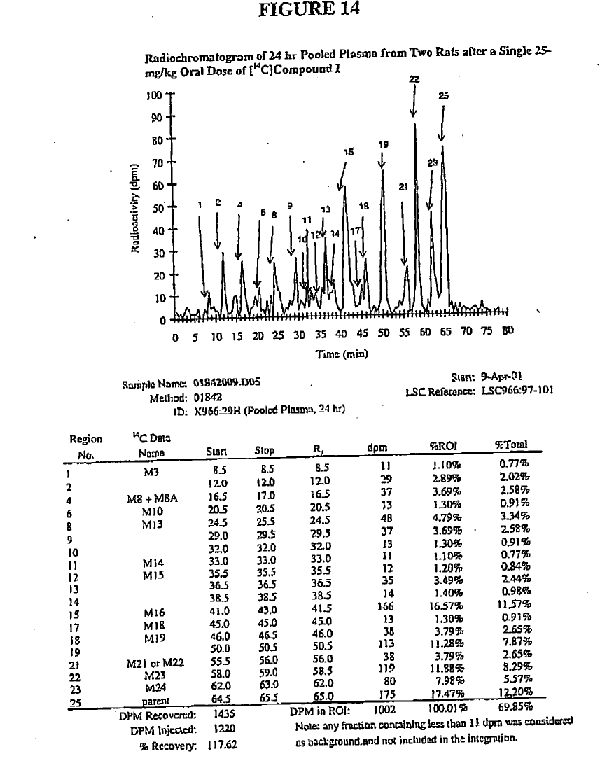

FIGS. 11 to 14 show the radiochromatograms of the acetonitile extract of pooled 1, 6, 12, and 24 hr plasma samples. The column recovery was quantitative for all four samples. The parent drug was a prominent radioactive component in plasma and was detectable up to 24 hr after dosing. At 24 hr, the concentration of Compound I in plasma was estimated to be 61 ng/mL. The profiles show more than 20 metabolites in plasma that were not analyzed by LC/RAM/(+)ESI-MS and MS/MS due to limited amount of material. These metabolites were identified based solely on their HPLC retention times compared to those metabolites identified in bile or urine. A total of 16 metabolites were tentatively identified in plasma. Together they accounted for 58.5% of total radioactivity in the 24 hr pooled plasma sample.

Metabolite Profiles in Bile and Urine

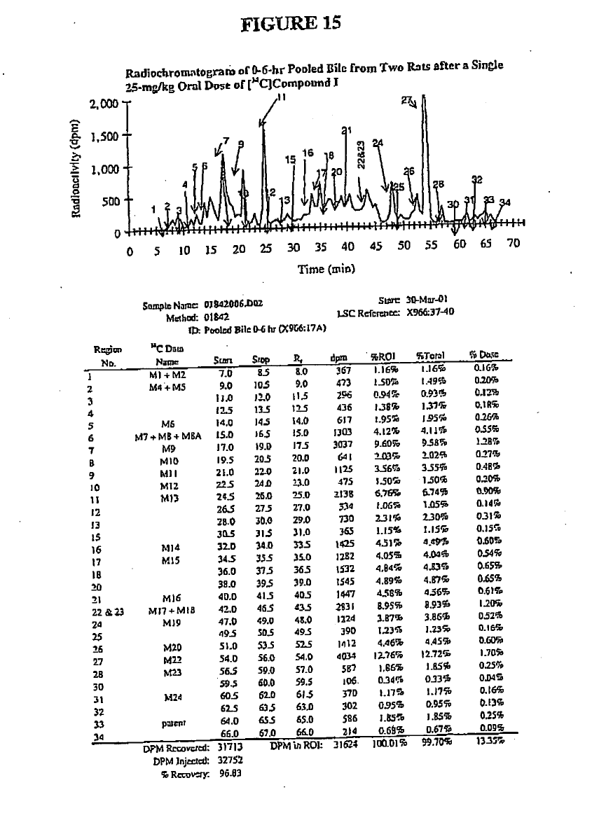

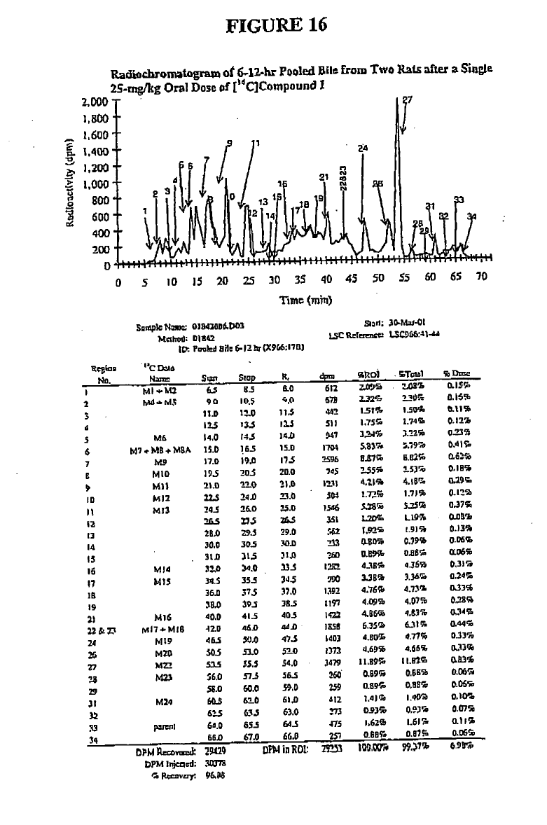

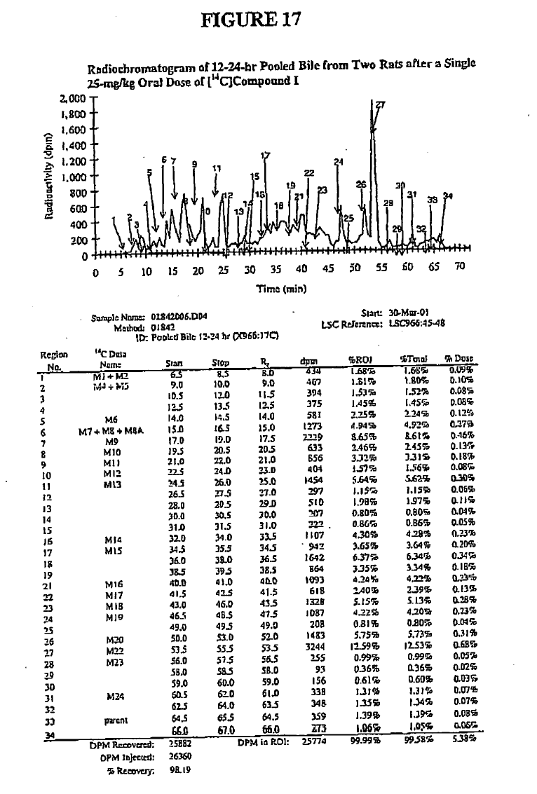

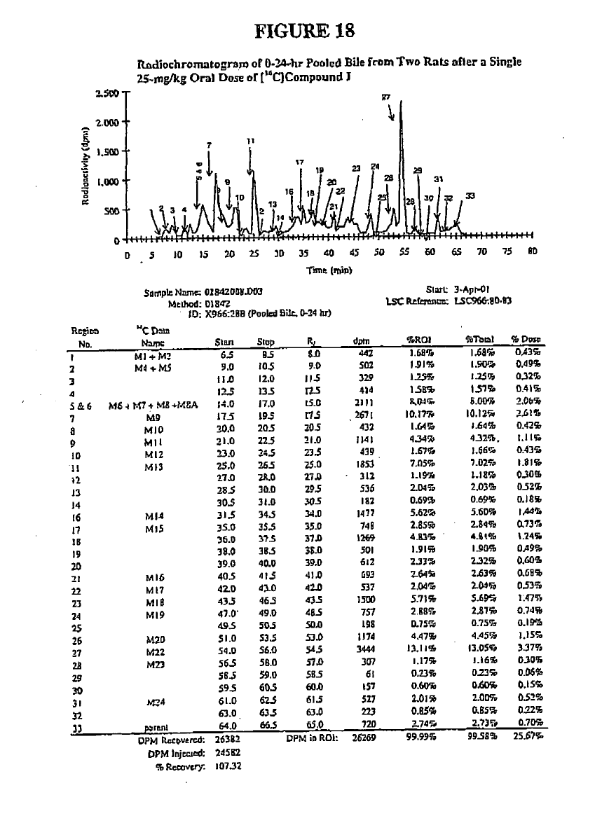

FIGS. 15-18 show the radiochromatograms of 0-6, 6-12, 12-24, and 0-24 hr pooled bile samples. The profiles show numerous radioactive components in bile that were not well resolved. No single major metabolite was found. The profiles were divided into regions, some of which may contain more than one radioactive compound. Little parent drug (<1% of total radioactivity) was detected (in 0-24 h bile). LC/RAM/(+)ESI-, LC/RAM/(−)ESI, and LC/RAM/APICD/(+)ESI-MS and MS/MS identified 23 metabolites in bile. Together they accounted for 79% of the total radioactivity (20.3% of the dose) in 0-24 hr pooled bile. Thirteen (M1-M13) of the 23 identified metabolites were products derived from hydrolysis of [¹⁴C] Compound I, namely the products derived from oxidation and/or glutathione conjugation of the baccatin moiety of [¹⁴C] Compound I. Oxidation took place primarily on the phenyl ring and the baccatin moiety. Glutathione conjugation occurred on the phenyl ring after the ring was oxidized.

Metabolites derived from the cleaved side chain of [¹⁴C] Compound I were not detected due to lack of radioactivity. These 13 metabolites derived from the baccatin moiety of [¹⁴C] Compound I together accounted for 36.3% of the total radioactivity (9.4% of the dose) in the pooled bile sample.

The other 10 metabolites identified in bile were the metabolites derived from oxidation and/or glutathione conjugation of [¹⁴C] Compound I. Together, they accounted for 42.3% of the total radioactivity (10.9% of the dose) in the 0-24 hr bile sanple. Oxidation of Compound I occurred primarily on the baccatin ring (the position of the oxidation could not be determined), and on one of the two t-butyl groups of the side chain. Two metabolites (M20 and M21) were identified as products derived from successive oxidations of the t-butyl group of the side chain followed by spontaneous cyclization of the putative aldehyde and acid derivatives. Similar cyclization metabolites of docetaxel were reported. Glutathione conjugation occurred on the benzoyl group after oxidation of the phenyl ring.

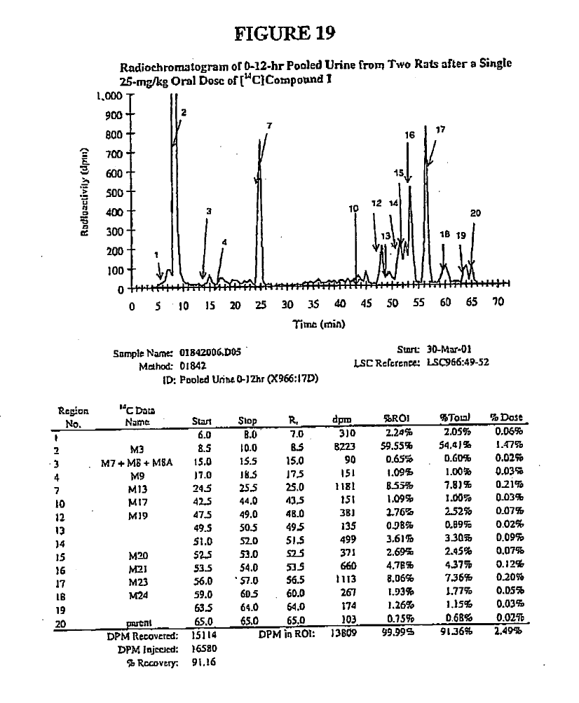

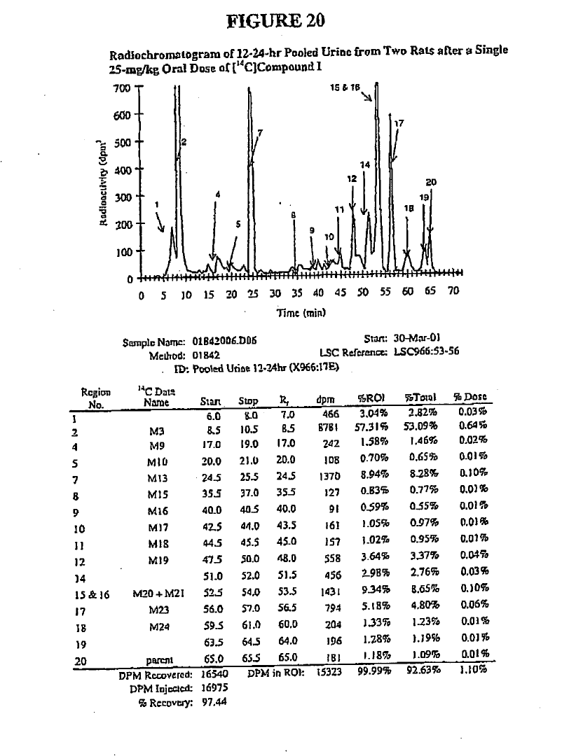

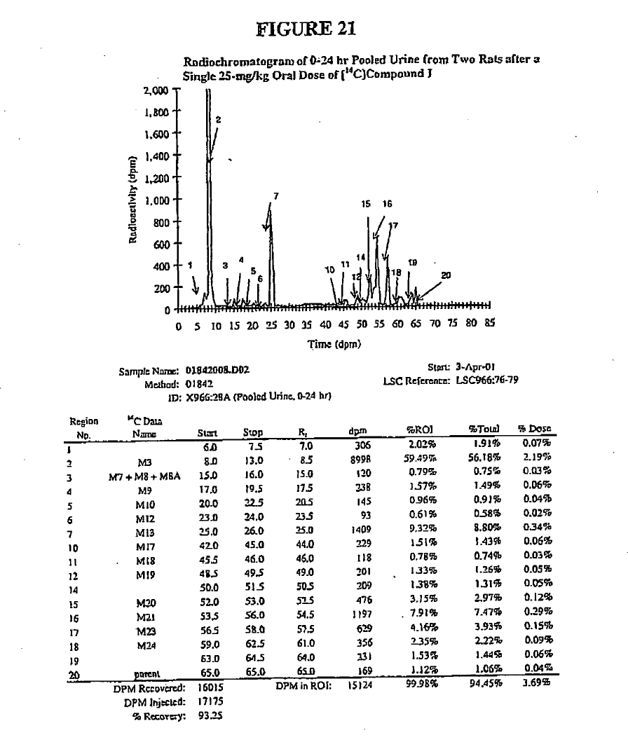

FIGS. 19-21 show the radiohromatograms of 0-12, 12-24, and 0-24 hr pooled urine samples. Little parent drug (≦1%) was detected in urine. Four metabolites in urine were identified or characterized by LC/RAM/(+)ESI-MS, LC/RAM/(−)ESI-MS and MS/MS as a hippuric acid (M3), a hydrolytic product (M13), and two oxidative products (M20 and M21) of [¹⁴C] Compound I. These four metabolites together accounted for 80% of the total radioactivity in pooled 0-24 hr urine or 3.1% of the dose. M3 (a hipporic acid), which was solely excreted in urine, alone accounted for ˜60% of the total radioactivity in 0-24 hr pooled urine or −2.3% of the dose. Twelve other minor metabolites were also identified in urine based on their retention times compared to those metabolites identified in bile.

### CONCLUSION

In 24 hr after a single 25-mg/kg oral dose of [¹⁴C] Compound I to two male BDC rats, averages of 25.8% and 3.9% of the dose were excreted in bile and urine, respectively. Total recovery of the adminstered dose averaged 29.6%. Thus, the oral absorption of [¹⁴C] Compound I in rats was estimated to be at least 30%. The parent drug was a prominent radioactive component in plasma and was measurable up to 24 hr after dosing (61 ng/mL). The absorbed drug was extensively metabolized and the formed metabolites were mainly excreted in bile. Little parent drug (≦1% of the dose) was detected in bile and urine.

HPLC radiochromatography revealed the formation of numerous metabolites. LC/RAM/(+)ESI- and LC/RAM/(−)ESI-MS and MS/MS analysis identified twenty-three metabolites in bile and four in urine. No single major metabolite was observed in bile. [¹⁴C]Hippuric acid (M3), which was solely excreted in urine, alone accounted for ˜60% of total radioactivity in the urine (0-24 hr) or −2.3% of the dose. The identified metabolites in bile and urine could be classified into two categories: one derived from cleavage of the ester side chain at the C₁₃-posifion of [¹⁴C] Compound I followed by oxidation and glutathione conjugation of the formed baccatin moiety (Category I metabolites), and the other derived from direct oxidation and glutathione conjugation of [¹⁴C] Compound I (Category II metabolites). The metaboliles of both categories were excreted in bile to about the same extent. Oxidation of [¹⁴C] Compound I occurred primarily on the baccatin moiety, the benzoyl group, and the two t-butyl groups of the side chain. Glutathione conjugation occurred primarily on the benzoyl group after the phenyl ring was oxidized. Two metabolites derived from successive oxidations of the t-butyl group of the side chain followed by spontaneous cyclization of the putative aldehyde and acid derivatives were identified.

The embodiments of the invention described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A metabolite of 3′-tert-butyl-3′-N-tert-butyloxycarbonyl-4-deacetyl-3′-dephenyl-3′-N-debenzoyl-4-O-methoxycarbonyl-paclitaxel of formula Ia or a pharmaceutically acceptable salt, solvate, hydrate or prodrug thereof 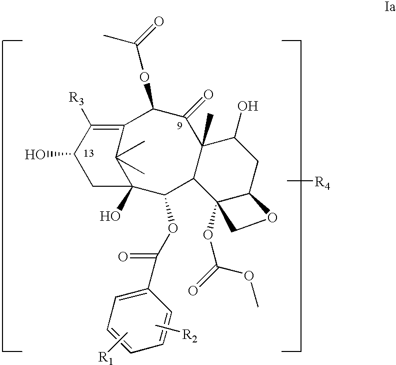
wherein the substituents are as defined in the following table

| Metabolite | | | | | |
|---|---|---|---|---|---|
| Code | R1 | R2 | R3 | R4 | C9 |
| M1 | SG | H | CH₃ | OH | C═O |
| M2 | SG | OH | CH₃ | H | C═O |
| M4 & M5 | SG | H | CH₃ | H | C═O |
| M6 | OCH₃ | OH | CH₃ | OH | C═O |
| M7 | OH | OCH₃ | CH₃ | OH | C═O |
| M8 | OH | H | CH₃ | OH | C═O |
| M8A | H | H | CH₃ | (OH)₂ | C═O |
| M9 | H | H | CH₃ | H | (CH)OH |
| M10 | H | H | CH₃ | OH | C═O |
| M11 | H | H | COOH | H | C═O |
| M12 | H | H | CH₃ | OH | C═O |
| M13 | H | H | CH₃ | H | C═O |

and

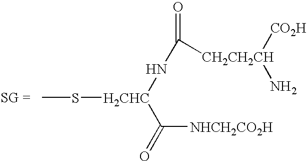

2. A metabolite of 3′-tert-butyl-3′-N-tert-butyloxycarbon-4-deacetyl-3′-dephenyl-3′-N-debenzoyl-4-O-methoxycarbonyl-paclitaxel of formula Ib or a pharmaceutically acceptable salt, solvate or prodrug thereof 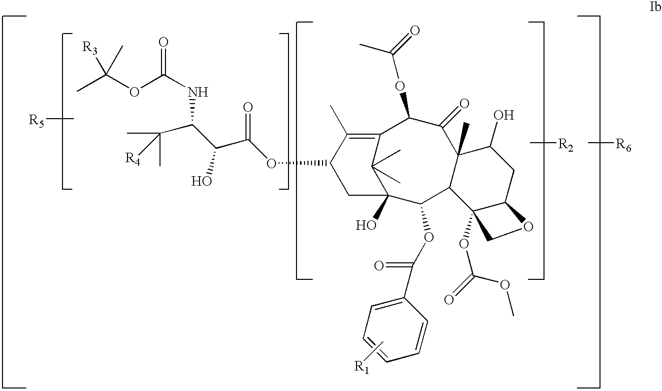
wherein the substituents are as defined in the following table

| Metabolite | | | | | | |
|---|---|---|---|---|---|---|
| Code | R₁ | R₂ | R₃ | R₄ | R₅ | R₆ |
| M14 | H | H | CH₃ | CH₃ | H | 2O |
| M15 | SG | H | CH₃ | CH₃ | H | H |
| M16 | H | H | CH₃ | CH₃ | H | 2O |
| M17 | H | H | CH₃ | CH₃ | OH | H |
| M18 | H | OH | CH₃ | CH₃ | OH | H |
| M19 | H | H | CH₃ | CH₃ | OH | H |
| M20 | H | H | | | | H |
| M21 | H | H | | | | H |
| M22 | H | H | CH₃ | COOH | H | H |
| M23 | H | H | CH₃ | CH₃ | OH | H |
| M24 | H | H | COOH | CH₃ | H | H |

and 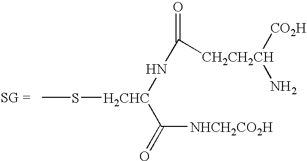
wherein the side chains on M20 and M21 are as shown below:

| | |
|---|---|
| M20 | 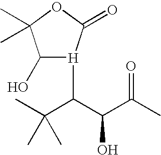 |
| | or |
| | 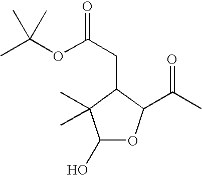 |
| M21 | 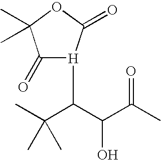 |
| | or |
| | 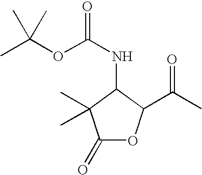 |

3. A metabolite of 3′-tert-butyl-3′-N-tert-butyloxycarbonyl-4-deacetyl-3′-dephenyl-3′-N-debenzoyl-4-O-methoxycarbonyl-paclitaxel of formula Ic or a pharmaceutically acceptable salt, solvate or prodrug thereof 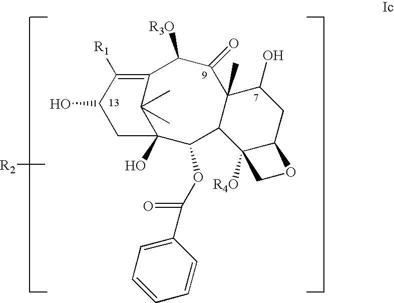
wherein the subsituents are as defined in the following table:

| Metabolite Code | R₁ | R₂ | R₃ | R₄ | C9 | C13 |
|---|---|---|---|---|---|---|
| M9 | CH₃ | H | CO(CH₃) | CO(OCH₃) | (CH)OH | (CH)OH |
| M10 | CH₃ | OH | CO(CH₃) | CO(OCH₃) | C═O | (CH)OH |
| M10A | CH₃ | H | H | CO(OCH₃) | C═O | (CH)OH |
| M11 | COOH | H | CO(CH₃) | CO(OCH₃) | C═O | (CH)OH |
| M10B | CH₃ | OH | CO(CH₃) | CO(OCH₃) | C═O | (CH)OH |
| M10C | CH₃ | H | CO(CH₂OH) | CO(OCH₃) | C═O | (CH)OH |
| M12 | CH₃ | OH | CO(CH₃) | CO(OCH₃) | C═O | (CH)OH |
| M13 | CH₃ | H | CO(CH₃) | CO(OCH₃) | C═O | (CH)OH |
| M13A | CH₃ | H | H | CO(OCH₃) | C═O | C═O |
| M13B | CH₃ | OH | CO(CH₃) | H | C═O | (CH)OH |
| M13C | CH₃ | H | CO(CH₃) | CO(OCH₃) | C═O | (CH)OH |
| M13D | CH₃ | H | CO(CH₃) | CO(OCH₃) | C═O | C═O |

4. A metabolite of 3′-tert-butyl-3′-N-tert-butyloxycarbonyl-4-deacetyl-3′-dephenyl-3′-N-debenzoyl-4-O-methoxycarbonyl-paclitaxel of formula Id or a pharmaceutically acceptable salt, solvate or prodrug thereof 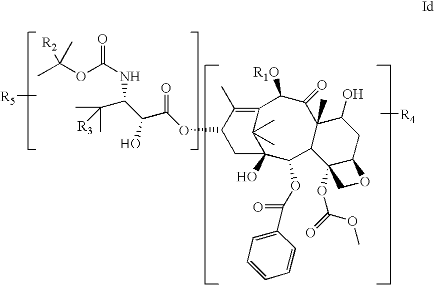
wherein the substituents are as defined in the following table:

| Metabolite | | | | | |
|---|---|---|---|---|---|
| Code | R₁ | R₂ | R₃ | R₄ | R₅ |
| M15B | CO(CH₃) | CH₂OH | COOH or | H | H |
| | | or | CH₂OH | | |
| | | COOH | | | |
| M17 | CO(CH₃) | CH₃ | CH₃ | H | OH |
| M18B | H | CH₃ | COOH | H | H |
| M19 | CO(CH₃) | CH₃ | CH₃ | H | OH |
| M19A | H | COOH | CH₃ | H | H |
| M22 | CO(CH₃) | CH₃ | COOH | H | H |
| M23 | CO(CH₃) | CH₃ | CH₃ | H | OH |
| M24 | CO(CH₃) | COOH | CH₃ or | H | H |
| | | or CH₃ | COOH | | |
| M23A | CO(CH₃) | CH₃ | CH₃ | OH or | H or |
| | | | | H | OH |
| M23B | CO(CH₃) | CH₃ | CH₃ | OH | H |
| M23C | CO(CH₃) | CH₃ | CH₃ | OH or | H or |
| | | | | H | OH |
| M26 | H | CH₃ | CH₃ | H | H |
| M23D | CO(CH₃) | CH₃ | CH₃ | OH | H |
| M27 | CO(CH₃) | CH₃ | CH₃ | H | H |

5. A pharmaceutical composition comprising a metabolite according to claim 1 or a pharmaceutically acceptable salt, solvate or prodrug thereof, and a pharmaceutically acceptable carrier, vehicle or diluent.

6. A pharmaceutical composition comprising a metabolite according to claim 2 or a pharmaceutically acceptable salt, solvate or prodrug thereof, and a pharmaceutically acceptable carrier, vehicle or diluent.

7. A pharmaceutical composition comprising a metabolite according to claim 3 or a pharmaceutically acceptable salt, solvate or prodrug thereof, and a pharmaceutically acceptable carrier, vehicle or diluent.

8. A pharmaceutical composition comprising a metabolite according to claim 4 or a pharmaceutically acceptable salt, solvate or prodrug thereof, and a pharmaceutically acceptable carrier, vehicle or diluent.

9. A method for inhibiting tumor growth in a mammalian host which comprises administering to said mammal a tumor-growth inhibiting amount of a compound as defined in claim 1.

10. A method for inhibiting tumor growth in a mammalian host which comprises administering to said mammal a tumor-growth inhibiting amount of a compound as defined in claim 2.

11. A method for inhibiting tumor growth in a mammalian host which comprises administering to said mammal a tumor-growth inhibiting amount of a compound as defined in claim 3.

12. A method for inhibiting tumor growth in a mammalian host which comprises administering to said mammal a tumor-growth inhibiting amount of a compound as defined in claim 4.
